(19) United States
(12) Patent Application Publication
(10) Pub. No.: US 2019/0316167 A1
(43) Pub. Date: Oct. 17, 2019

(54) Methods for Treating and Detecting Johne's Disease in Cattle

(71) Applicant: CNA Diagnostics Inc., Calgary (CA)

(72) Inventors: Christoph W. Sensen, Graz (AT); Jung Soh, Graz (AT); Petra Heidinger, Graz (AT); Laura Villanova, Graz (AT); Stefan Grabuschnig, Graz (AT)

(73) Assignee: CNA Diagnostics Inc., Calgary (CA)

(21) Appl. No.: 16/385,970

(22) Filed: Apr. 16, 2019

Related U.S. Application Data
(60) Provisional application No. 62/658,297, filed on Apr. 16, 2018.

(51) Int. Cl. C12Q 1/04; C12Q 1/686; A61K 31/7048; C12Q 1/6806; C12Q 1/6874; C12Q 1/6837; A61P 31/04

(52) U.S. Cl. CPC C12Q 1/04; C12Q 1/686; A61K 31/7048; C12Q 1/6806; C12Q 2600/16; C12Q 1/6837; A61P 31/04; C12Q 2600/166; C12Q 2600/112; C12Q 1/6874

(57) ABSTRACT

Biomarkers for identifying Johne's disease in cattle are presented herein, as are related methods, uses, agents, and kits comprising same. Methods for treating, detecting, quarantining, and diagnosing Johne's disease in cattle are presented herein.

### RELATED APPLICATIONS

This application claims priority of U.S. Provisional Application No. 62/658,297 filed Apr. 16, 2018, the entirety of which is incorporated herein by reference for all purposes.

### SEQUENCE LISTING

The instant application contains a Sequence Listing which has been submitted electronically in ASCII format and is hereby incorporated by reference in its entirety. Said ASCII copy, created on Apr. 8, 2019, is named 176395-010301_PCT_SL.txt and is 56,438 bytes in size.

### FIELD OF INVENTION

Polynucleotides relating to circulating nucleic acids (CNAs) indicative of Johne's disease (JD) are described herein, as are methods and kits for detecting and diagnosing JD in cattle.

### BACKGROUND

JD is a chronic, contagious granulomatous enteritis that affects predominantly ruminants, including cattle, sheep, goats, bison, llamas, deer, and elk. JD is also referred to as paratuberculosis. It is listed as a priority disease for international trade by the World Organization for Animal Health. In cattle, JD is characterized by persistent diarrhea, progressive weight loss, debilitation, and eventually death. The etiologic agent, Mycobacterium paratuberculosis, also known as Mycobacterium avium sub sp paratuberculosis (MAP), infects and causes disease in all ruminants and is also thought to infect and cause disease in other animals, both domestic and free-ranging. MAP infection has also been detected in omnivores and carnivores such as pigs, nonhuman primates, wild rabbits, foxes, and weasels. JD is worldwide concern. Indeed, estimates suggest that up to 77% of the North American herds are infected by MAP. National control programs to limit MAP infection have been established in Australia, Norway, Iceland, Japan, the Netherlands, Denmark, Ontario, Canada, and the USA. The highest published prevalence is in dairy cattle, with 20%-85% of herds infected in many of the major dairy-producing countries. The disease is of economic importance for the goat industry in Spain and the sheep industry in Australia.

### SUMMARY

Methods, reagents, and kits described herein relate to treating, quarantining, predicting and/or diagnosing JD in a bovine animal in advance of the appearance of JD symptoms in the bovine animal. In accordance with the present experimental findings, methods, reagents, and kits described herein can be used to treat/quarantine/predict/diagnose JD in a bovine subject in advance of symptomatic presentation, as well as in later stages of the disease. Indeed, results presented herein demonstrate that methods, reagents, and kits described herein can diagnose JD in a bovine subject well in advance of clinical presentation. In light of results presented in the examples, over-representation or under-representation of at least one polynucleotide relative to an internal reference region in a biological sample or body fluid sample (e.g., serum), wherein the at least one polynucleotide comprises any one of SEQ ID NOs: 1-16 or 134-164, is a positive indicator that a bovine subject from which the sample was isolated will develop JD. Accordingly, the methods, reagents, and kits described herein provide for diagnosis of JD at early, pre-symptomatic stages of the disease, as well as later stages of the disease. Definitive diagnosis of JD permits selection of infected animals for suitable treatment regimens, quarantine, and if necessary, slaughter. Methods for determining over-representation or under-representation of at least one polynucleotide (CNA) relative to an internal reference region, wherein the at least one polynucleotide comprises any one of SEQ ID NOs: 1-16 or 134-164, in body fluids are also disclosed, as are kits for such purposes.

In a particular aspect, a method for treating a bovine animal suspected of having Johne's disease is presented, the method comprising treating the bovine animal identified as having Johne's disease with a therapeutically effective amount of at least one agent used to treat Johne's disease, wherein the bovine animal is identifiable as having Johne's disease by analyzing a biological sample isolated from the bovine animal for over-representation or under-representation of at least one polynucleotide relative to an internal reference region, wherein the at least one polynucleotide comprises any one of SEQ ID NOs: 1-16 or 134-164 and wherein the over-representation or under-representation of the at least one polynucleotide in the biological sample is a positive indicator of Johne's disease.

Also encompassed herein is use of a therapeutically effective amount of at least one agent used to treat Johne's disease to treat Johne's disease in a bovine animal identifiable as having Johne's disease by analyzing a biological sample isolated from the bovine animal for over-representation or under-representation of at least one polynucleotide relative to an internal reference region, wherein the at least one polynucleotide comprises any one of SEQ ID NOs: 1-16 or 134-164 and wherein the over-representation or under-representation of the at least one polynucleotide in the biological sample is a positive indicator of Johne's disease.

In another aspect, a method for treating a bovine animal identified as exhibiting over-representation or under-representation of at least one polynucleotide relative to an internal reference region is presented, wherein the at least one polynucleotide comprises any one of SEQ ID NOs: 1-16 or 134-164 and wherein the over-representation or the under-representation of the at least one polynucleotide in the biological sample is a positive indicator of Johne's disease, the method comprising treating the bovine animal identified as exhibiting the over-representation or the under-representation of the at least one polynucleotide relative to the internal reference region with a therapeutically effective amount of at least one agent used to treat Johne's disease.

In yet another aspect, a method for treating a bovine animal at risk for developing Johne's disease is presented, the method comprising treating the bovine animal at risk for developing Johne's disease with a therapeutically effective amount of at least one agent used to treat Johne's disease, wherein the bovine animal is identifiable as at risk for developing Johne's disease by analyzing a biological sample isolated from the bovine animal for over-representation or under-representation of at least one polynucleotide relative to an internal reference region, wherein the at least one polynucleotide comprises any one of SEQ ID NOs: 1-16 or 134-164 and wherein the over-representation or under-representation of the at least one polynucleotide in the biological sample is a positive indicator of Johne's disease.

In a further aspect, a method for detecting Johne's disease in a bovine animal is presented, comprising

(a) analyzing a biological sample isolated from the bovine animal for over-representation or under-representation of at least one polynucleotide relative to an internal reference region, wherein the at least one polynucleotide comprises any one of SEQ ID NOs: 1-16 or 134-164, by contacting the biological sample with at least one synthetic probe specific for a polynucleotide comprising any one of SEQ ID NOs: 1-16 or 134-164, wherein the contacting generates complexes of synthetic probes bound to specific polynucleotides when at least one polynucleotide comprising any one of SEQ ID NOs: 1-16 or 134-164 is present in the biological sample,

(b) detecting the complexes of synthetic probes bound to specific polynucleotides, and detecting the internal reference region in the biological sample, and

(c) comparing the at least one polynucleotide comprising any one of SEQ ID NOs: 1-16 or 134-164 detected in the biological sample to the internal reference region detected in the biological sample to determine relative over-representation and under-representation of the at least one polynucleotide in the biological sample, wherein detection of the over-representation and under-representation of the at least one polynucleotide serves as a positive indicator of Johne's disease in the bovine animal.

In another aspect, a method for quarantining a bovine animal identified as exhibiting over-representation or under-representation of at least one polynucleotide relative to an internal reference region is presented, wherein the at least one polynucleotide comprises any one of SEQ ID NOs: 1-16 or 134-164 and wherein the over-representation or the under-representation of the at least one polynucleotide in the biological sample is a positive indicator of Johne's disease, the method comprising

a) quarantining the bovine animal identified as exhibiting the over-representation or the under-representation of the at least one polynucleotide relative to the internal reference region, and

b) optionally, at least one of treating the quarantined bovine animal with a therapeutically effective amount of at least one agent used to treat Johne's disease and slaughtering the quarantined bovine animal.

In accordance with any of the methods described herein, the at least one polynucleotide comprising any one of SEQ ID NOs: 1-16 or 134-164 over-represented or under-represented relative to the internal reference region is at least two, at least three, at least four, or at least five of the polynucleotides comprising any one of SEQ ID NOs: 1-16 or 134-164. Exemplary internal reference regions are presented in Table 1 (SEQ ID NOs: 17-56) and over-representation and under-representation may be determined relative to the level of at least one of the internal reference regions. An internal standard region may also represent a composite of at least 2, 3, 4, 5, 6, 7, 8, 9, 10, 11, 12, 13, 14, 15, 16, 17, 18, 19, 20, 25, 30, 35, 40, 45, or 50 or more individual internal standard regions.

In an embodiment, a bovine animal is identifiable as having Johne's disease by analysis of a biological sample isolated from the bovine animal for the over-representation or the under-representation of the at least one polynucleotide relative to the internal reference region.

In another aspect, a method for evaluating representation of at least one polynucleotide comprising any one of SEQ ID NOs: 1-16 or 134-164 in a biological sample of a bovine animal is presented, the method comprising:

analyzing the biological sample of the bovine animal for over-representation or under-representation of at least one polynucleotide relative to an internal reference region, wherein the at least one polynucleotide comprises any one of SEQ ID NOs: 1-16 or 134-164, and wherein the over-representation or the under-representation of the at least one polynucleotide in the biological sample is determined by detecting the at least one polynucleotide comprising any one of SEQ ID NOs: 1-16 or 134-164 in the biological sample, wherein the detecting is achieved by contacting the biological sample with at least one reagent that specifically binds to any one of SEQ ID NOs: 1-16 or 134-164, and detecting the internal reference region in the biological sample, and comparing the at least one polynucleotide comprising any one of SEQ ID NOs: 1-16 or 134-164 detected in the biological sample to the internal reference region detected in the biological sample to determine relative over-representation and under-representation of the at least one polynucleotide in the biological sample.

In accordance with any of the methods described herein, the biological sample is blood, a product derived from blood, or a fraction derived from blood. In an embodiment thereof, the product derived from blood is plasma or serum.

In accordance with any of the methods described herein, detecting the over-representation or under-representation of the at least one polynucleotide relative to an internal reference region comprises at least one of a polymerase chain reaction (PCR)-based detection method, a hybridization-based method, enzyme-linked immunosorbent assay (ELISA), radioimmunoassay (RIA), solid-phase enzyme immunoassay (EIA), mass spectrometry, and microarray analysis. In a particular embodiment thereof, the PCR-based detection method comprises amplifying nucleic acid sequences in the biological sample using primers that are specific for and capable of amplifying any one of SEQ ID NOs: 1-16 or 134-164, wherein the amplifying generates amplification products corresponding to any one of SEQ ID NOs: 1-16 or 134-164 when the biological sample comprises any one of SEQ ID NOs: 1-16 or 134-164. In a more particular embodiment thereof, the PCR-based detection method is performed using at least one primer pair, wherein each primer pair of the at least one primer pair is specific for any one of SEQ ID NOs: 1-16 or 134-164. In a still more particular embodiment thereof, the primer pair specific for any one of SEQ ID NOs: 1-16 or 134-164 is any one of the primer pairs presented in Table 1. In a particular embodiment, amplification products corresponding to any one of SEQ ID NOs: 1-16 or 134-164 may be sequenced.

In an embodiment of any of the methods described herein, the nucleic acid sequences comprise circulating nucleic acids.

In an embodiment of any of the methods described herein involving treatment, the at least one agent used to treat Johne's disease comprises at least one of antibiotic. In a particular embodiment, the at least antibiotic is in the rifabutin genus. In a more particular embodiment, the at least antibiotic is in the clarithromycin genus. In a still further embodiment, the at least one antibiotic is rifabutin and clarithromycin.

In an embodiment of any of the methods described herein, the bovine animal is monitored for Johne's disease. Such monitoring may be a matter of routine maintenance of a herd, a follow up to suspected or potential exposure to an infected animal or by-products thereof, a follow up to a known exposure to an infected animal or by-products thereof, or monitoring disease status of a sick animal.

In an aspect of methods described herein, the over-representation or under-representation of the at least one polynucleotide relative to an internal reference region is determined using reagents comprising an antibody or a nucleic acid probe specific for any one of SEQ ID NOs: 1-16 or 134-164. In an embodiment thereof, the antibody is a monoclonal or a polyclonal antibody. In a more embodiment thereof, the antibody is obtained from mice, rats, rabbits, goats, chicken, donkey, horses or guinea pigs.

In another aspect of methods described herein, the over-representation or under-representation of the at least one polynucleotide relative to an internal reference region is determined using reagents comprising a nucleic acid probe specific for any one of SEQ ID NOs: 1-16 or 134-164 that may, for example, be labeled with a detectable label.

Also encompassed herein is a probe comprising a manmade nucleotide sequence capable of binding specifically to a polynucleotide comprising any one of SEQ ID NOs: SEQ ID NOs: 1-56 and SEQ ID NOs: 134-164 and at least one manmade tag conjugated thereto, wherein the manmade nucleotide sequence is complementary to the polynucleotide comprising any one of SEQ ID NOs: SEQ ID NOs: 1-56 and SEQ ID NOs: 134-164. In an embodiment thereof, the manmade nucleotide sequence capable of binding specifically to a polynucleotide comprising any one of SEQ ID NOs: SEQ ID NOs: 1-56 and SEQ ID NOs: 134-164 exhibits at least 90%, at least 91%, at least 92%, at least 93%, at least 94%, or at least 95%, at least 96%, at least 97%, at least 98%, or at least 99% complementarity to any one of SEQ ID NOs: SEQ ID NOs: 1-56 and SEQ ID NOs: 134-164. In a particular embodiment thereof, the manmade tag is a detectable marker. In a more particular embodiment thereof, the detectable marker comprises a radioactive marker or fluorescent marker.

In another aspect, an array comprising at least one probe described herein is presented, wherein the at least one probe is bound to a solid surface.

In yet another aspect, a kit comprising at least one probe described herein is presented, as well as instructions for use thereof.

In a further aspect thereof, an array or kit presented herein comprises at least four or at least eight different probes comprising a manmade nucleotide sequence capable of binding specifically to a polynucleotide comprising any one of SEQ ID NOs: SEQ ID NOs: 1-56 and SEQ ID NOs: 134-164, wherein optionally, at least one manmade tag conjugated to each manmade nucleotide sequence, wherein the manmade nucleotide sequence is complementary to the polynucleotide comprising any one of SEQ ID NOs: SEQ ID NOs: 1-56 and SEQ ID NOs: 134-164.

In a particular embodiment, an array described herein is a microarray, gene chip, DNA chip, or a FILMARRAY®.

In another aspect, a primer comprising a manmade nucleotide sequence capable of binding specifically to a polynucleotide comprising any one of SEQ ID NOs: SEQ ID NOs: 1-56 and SEQ ID NOs: 134-164 having at least one manmade tag conjugated thereto is presented, wherein the manmade nucleotide sequence is any one of the polynucleotide sequences listed in Table 1 or a variant thereof. In an embodiment thereof, the variant of any one of the polynucleotide sequences listed in Table 1 is at least 90%, at least 91%, at least 92%, at least 93%, at least 94%, or at least 95%, at least 96%, at least 97%, at least 98%, or at least 99% identical to any one of the polynucleotide sequences listed in Table 1. In another embodiment thereof, the manmade tag is a detectable marker. In a more particular embodiment thereof, the detectable marker comprises a radioactive marker or fluorescent marker.

In yet another aspect, a primer consisting essentially of or consisting of a manmade nucleotide sequence capable of binding specifically to a polynucleotide comprising any one of SEQ ID NOs: SEQ ID NOs: 1-56 and SEQ ID NOs: 134-164 and at least one manmade tag conjugated thereto is presented, wherein the manmade nucleotide sequence is any one of the polynucleotide sequences listed in Table 1 or a variant thereof. In an embodiment thereof, the variant of any one of the polynucleotide sequences listed in Table 1 is at least 90%, at least 91%, at least 92%, at least 93%, at least 94%, or at least 95%, at least 96%, at least 97%, at least 98%, or at least 99% identical to any one of the polynucleotide sequences listed in Table 1. In a particular embodiment, a variant of a primer comprises different nucleotides at the 5′ end of the primer, which positions are more tolerant of variations thereto. In a particular embodiment, the manmade tag is a detectable marker (e.g., a radioactive marker, fluorescent dye, tag that is specifically recognized (e.g., bound) by a labeled reagent (e.g., a labeled antibody), a tag that is specifically bound by a magnetic bead, or any other marker comprising detectable label.

Also encompassed herein is a primer consisting essentially of or consisting of a manmade nucleotide sequence capable of binding specifically to a polynucleotide comprising any one of SEQ ID NOs: SEQ ID NOs: 1-56 and SEQ ID NOs: 134-164 and at least one manmade tag conjugated thereto, wherein the manmade nucleotide sequence is any one of the polynucleotide sequences listed in Table 1 or a variant thereof. In an embodiment thereof, the variant of any one of the polynucleotide sequences listed in Table 1 is at least 90%, at least 91%, at least 92%, at least 93%, at least 94%, or at least 95%, at least 96%, at least 97%, at least 98%, or at least 99% identical to any one of the polynucleotide sequences listed in Table 1. In a particular embodiment, the manmade tag is a detectable marker (e.g., a radioactive marker or fluorescent marker).

Also encompassed herein are polynucleotides comprising each of SEQ ID NOs: 1, 3, 5, 7, 9, 11, 13, 15, 17, 19, 21, 23, 25, 27, 29, 31, 33, 35, 37, 39, 41, 43,45, 47, 49, 51, 53, and 55, which include additional 5′ and 3′ sequences that flank each of these sequenves in the bovine genome. These larger sequences, which include flanking sequences, are designated SEQ ID NOs: 2, 4, 6, 8, 10, 12, 14, 16, 18, 20, 22, 24, 26, 28, 30, 32, 34, 36, 38, 40, 42, 44, 46, 48, 50, 52, 54, and 56. Primer pairs complementary to any one of SEQ ID NOs: SEQ ID NOs: 1-56 and SEQ ID NOs: 134-164 and suitable for PCR amplification are readily determined based on the sequences of SEQ ID NOs: SEQ ID NOs: 1-56 and SEQ ID NOs: 134-164 (5′ to 3′ strands) and reverse strands thereof (3′ to 5′ strands). Such primers are typically 8-20 nucleotides in length and are complementary (e.g., exhibit perfect complementarity or may be variants thereof that maintain a degree of complementarity sufficient to bind and act as primers in a PCR amplification) to any one of SEQ ID NOs: SEQ ID NOs: 1-56 and SEQ ID NOs: 134-164 or a reverse strand thereof. Choices regarding primer pairs suitable for PCR amplification are also determined based on the distance between the primers in a pair, with the understanding that PCR amplification products must be of a detectable size.

In another embodiment, therapeutic efficacy of a treatment regimen may be evaluated based on a change in the over-representation or under-representation of at least one of SEQ ID NOs: 1-16 or 134-164 following onset of the treatment regimen. In a particular embodiment, a decrease in representation of a CNA that is over-represented in JD (e.g., SEQ ID NOs: 1-14; 134-144; and 157-160) is indicative that the treatment regimen is therapeutically effective. In another particular embodiment, an increase in representation of a CNA that is under-represented in JD (e.g., SEQ ID NOs: 15-16; 145-156; and 161-164) is indicative that the treatment regimen is therapeutically effective.

In a further aspect, an array comprising at least one primer listed in Table 1 is presented, wherein the at least one primer is bound to a solid surface.

In a still further aspect, a kit for detecting JD in a bovine animal comprising at least one primer pair for amplifying a polynucleotide comprising any one of SEQ ID NOs: 1-16 or 134-164 is presented, wherein the at least one primer pair is listed in Table 1 and the kit includes instructions for use thereof. In an embodiment thereof, the kit comprises at least four primer pairs, wherein each primer pair of the four primer pairs specifically amplifies a different polynucleotide comprising any one of SEQ ID NOs: 1-16 or 134-164. In another embodiment thereof, the kit comprises at least eight primer pairs, wherein each primer pair of the eight primer pairs specifically amplifies a different polynucleotide comprising any one of SEQ ID NOs: 1-16 or 134-164.

In another aspect, use of a therapeutically effective amount of at least one agent used to treat Johne's disease to treat a bovine animal (e.g., a suitable antibiotic) is presented, wherein the bovine animal is identifiable as having Johne's disease by analyzing a biological sample isolated from the bovine animal for over-representation or under-representation of at least one polynucleotide relative to an internal standard region, wherein the at least one polynucleotide comprises any one of SEQ ID NOs: 1-16 or 134-164 and wherein the over-representation or under-representation of the at least one polynucleotide in the biological sample is a positive indicator of Johne's disease.

In another aspect, use of a therapeutically effective amount of at least one agent used to treat Johne's disease (e.g., a suitable antibiotic) in the preparation of a medicament for treating a bovine animal is presented, wherein the bovine animal is identifiable as having Johne's disease by analyzing a biological sample isolated from the bovine animal for over-representation or under-representation of at least one polynucleotide relative to an internal standard region, wherein the at least one polynucleotide comprises any one of SEQ ID NOs: 1-16 or 134-164 and wherein the over-representation or under-representation of the at least one polynucleotide in the biological sample is a positive indicator of Johne's disease.

In an embodiment of the use, the at least one polynucleotide comprising any one of SEQ ID NOs: 1-16 or 134-164 is over-represented or under-represented relative to the internal standard region is at least two, at least three, at least four, or at least five of the polynucleotides comprising any one of SEQ ID NOs: 1-16 or 134-164.

In an embodiment of the use, the bovine animal is identifiable as having Johne's disease by analysis of a biological sample isolated from the bovine animal for the over-representation or the under-representation of the at least one polynucleotide relative to the internal standard region.

In an embodiment of the use, the biological sample is blood, a product derived from blood, or a fraction derived from blood. In a particular embodiment, the product derived from blood is plasma or serum.

In an embodiment of the use, detecting the over-representation or under-representation of the at least one polynucleotide relative to an internal standard region comprises at least one of a polymerase chain reaction (PCR)-based detection method, a hybridization-based method, enzyme-linked immunosorbent assay (ELISA), radioimmunoassay (RIA), solid-phase enzyme immunoassay (EIA), mass spectrometry, and microarray analysis. In a particular embodiment, the PCR-based detection method comprises amplifying nucleic acid sequences in the biological sample using primers that are specific for and capable of amplifying any one of SEQ ID NOs: 1-16 or 134-164, wherein the amplifying generates amplification products corresponding to any one of SEQ ID NOs: 1-16 or 134-164 when the biological sample comprises any one of SEQ ID NOs: 1-16 or 134-164. In a more particular embodiment, the PCR-based detection method is performed using at least one primer pair, wherein each primer pair of the at least one primer pair is specific for any one of SEQ ID NOs: 1-16 or 134-164. In a still more particular embodiment, the primer pair specific for any one of SEQ ID NOs: 1-16 or 134-164 is any one of the primer pairs presented in Table 1.

In a particular embodiment, the use further comprises sequencing the amplification products corresponding to any one of SEQ ID NOs: 1-16 or 134-164.

In a particular embodiment of the use, the at least one polynucleotide is a nucleic acid sequence comprising a circulating nucleic acid.

In a particular embodiment of the use, the at least one agent used to treat Johne's disease or used in the preparation of a medicament comprises at least one of antibiotic. In a particular embodiment, the at least antibiotic is in a genus comprising rifabutin. In another particular embodiment, the at least antibiotic is in a genus comprising clarithromycin. In a still particular embodiment, the at least antibiotic is rifabutin and clarithromycin.

In a particular embodiment of the use, the bovine animal is monitored for Johne's disease.

Additional aspects of the present invention will be apparent in view of the description which follows.

### BRIEF DESCRIPTION OF THE FIGURES AND TABLES

The invention will now be described in relation to the tables.

Table 1 presents nucleic acid sequences for CNAs corresponding to either a CNA that is over-represented or under-represented in animals infected with MAP (SEQ ID NOs: 1-16 or 134-164) or an internal reference region that does not vary in MAP-infected or uninfected animals (SEQ ID NOs: 17-56.

FIG. 1 presents an exemplary Johne's disease algorithm workflow.

### DETAILED DESCRIPTION OF THE INVENTION

Various embodiments will be described in detail with reference to the drawings and tables. Reference to various embodiments does not limit the scope of the claims attached hereto. Additionally, any examples set forth in this specification are not intended to be limiting and merely set forth some of the many possible embodiments.

JD/paratuberculosis is typically fatal disease of ruminants that can remain undetected for years in asymptomatic carriers, until they succumb to the disease and present clinically. Although largely asymptomatic, these carriers can spread JD among other herd members.

Accordingly, JD has serious economic consequences because a large percent of a given herd can become infected prior to any visible means of detecting the disease. MAP is present at high titers in the feces of infected animals and at lower titers in colostrum and milk. MAP is durable and largely resistant to environmental factors. MAP can survive on a pasture or in water for >1 year. Durability of MAP is likely due to its ability to form spores. The infection is typically acquired via the fecal-oral route, although it can be transmitted in utero and via nursing. Introduction of the disease into a non-infected herd is usually through herd expansion or replacement purchases. Infection is introduced via subclinically infected carriers. The long subclinical phase of MAP-induced disease makes it difficult to screen for the presence of infected animals.

Infection is typically acquired early in life, but clinical signs rarely develop in cattle before the age of two because progression to clinical disease is slow. MAP infection typically occurs in bovine animals within the first year of life, when animals are most susceptible to MAP infection, and likely occurs shortly after birth. Resistance to infection increases with age and cattle exposed as adults are much less likely to become infected. Infection occurs via ingestion of MAP when nursing on contaminated teats; consumption of milk, solid feed, or water contaminated by the organism; or licking and grooming behavior in a contaminated environment. After ingestion and uptake in the Peyer's patches of the lower small intestine, MAP infects macrophages in the gastrointestinal (GI) tract and associated lymph nodes. In that MAP is an intracellular pathogen, it proliferates inside infected macrophages, thereby creating reservoirs for ongoing infection. Infected macrophages eventually die and release high titer levels of MAP and/or are taken up by other phagocytic cells, thereby spreading MAP infection to other cells. MAP infection eventually provokes a chronic granulomatous enteritis that interferes with nutrient uptake and processing, leading to the cachexia typical of advanced infections. JD progression may take months to years to develop and is usually accompanied by a decline in cell-mediated immunity, a rise in serum antibody, and bacteremia once the infection is disseminated beyond the GI tract. Fecal shedding begins before clinical signs are apparent and animals in sub-clinical stages of infection are significant sources of transmission.

paratuberculosis in cattle (bovine animals) is characterized by weight loss and diarrhea in the late phases of infection, but infected animals can appear healthy for months to years. In cattle, diarrhea may be constant or intermittent; in sheep, goats, and other ruminants, diarrhea may not be seen. The diarrhea typically does not contain blood, mucus, or epithelial debris. As the diseases progresses, the diarrhea becomes more severe, further weight loss occurs, coat color may fade, and ventral and intermandibular edema may develop due to the loss of protein via enteropathy. In dairy cattle and goats, milk yield may drop or fail to reach expected levels. Animals are remain alert and are typically normal with respect to body temperature and appetite during phases of disease characterized by diarrhea. The disease is progressive and ultimately terminates in emaciation and death. In infected herds, the initial mortality rate may be low for a number of years, but as many as 50% of the animals may be infected subclinically in such herds and therefore, likely to succumb to disease eventually. The disease in sheep and goats is similar, but diarrhea is not a common feature. Advanced cases in sheep and goats typically shed wool easily.

Prior to the discoveries described herein, only two kinds of tests were used to diagnose JD. These tests rely on ELISA or PCR reagents and test kits to detect the presence of MAP in cattle by detecting MAP specific products. In most cases, these tests analyze feces or bodily fluids (such as milk) for the presence of MAP sequences or proteins. These tests are suboptimal, however, particularly with respect to the ability to detect MAP during early stages of infection and for animals not shedding bacteria into the environment. The lack of sensitivity of these conventional tests is evident when compared to the sensitivity of the methods described herein. The present inventors used the DNA sequence data set described herein to identify five animals as positive for MAP infection that the conventional prior art tests incorrectly identified as healthy animals. The lack of sensitivity and selectivity of the existing conventional Johne's testing assays is known in the industry, and may account, at least in part, for the low number of tests performed (approximately 25,000 per annum in Canada and Austria, respectively).

Further to the above, conventional Johne's testing assays are directed to detecting the presence of the infective agent (MAP). Accordingly, these technologies are limited to those stages of disease wherein larger quantities of MAP are shed by the animals. In contrast, methods described herein are based on measurements of the host response to the MAP challenge, which is less dependent on MAP levels in the animal. The present reagents and methods, therefore, exhibit higher selectivity and sensitivity than the conventional Johne's testing assays and, furthermore, facilitate diagnosis of JD at earlier, pre-clinical stages of MAP infection.

In general, the present disclosure describes polynucleotide sequences of circulating nucleic acids (CNA) isolated from blood serum of MAP-infected cattle. The polynucleotide sequences are over-represented or under-represented relative to an internal reference region in MAP-infected animals and can be used to detect MAP-infected animals before these animals display clinical symptoms of JD. Assays and methods comprising the polynucleotide sequences and/or primers and/or probes for amplifying or detecting the polynucleotide sequences in a sample (e.g., blood serum) isolated from cattle are also described and are useful for screening a broad range of cattle breeds for MAP infection.

Methods, reagents, and kits described herein relate to treating, quarantining, predicting and/or diagnosing JD in a bovine animal in advance of the appearance of symptoms of JD in the animal. In accordance with the experimental findings presented herein, methods, reagents, and kits described herein are useful for diagnosing JD in a bovine animal in advance of symptomatic presentation. Indeed, results presented herein demonstrate that methods, reagents, and kits described herein can diagnose JD in a bovine animal well in advance (e.g., years in advance) of clinical presentation. In light of results presented in the Examples, detection of over-representation or under-representation of at least one polynucleotide relative to an internal reference region in a body fluid sample (e.g., serum) isolated from a bovine animal, wherein the at least one polynucleotide comprises any one of SEQ ID NOs: 1-16 or 134-164, is a positive indicator that the bovine animal will develop JD symptoms. Accordingly, the methods, reagents, and kits described herein provide for diagnosis of JD at pre-clinical stages (asymptomatic stages) of the disease. Also encompassed herein are methods to assess over-representation or under-representation of at least one polynucleotide relative to an internal reference region, wherein the at least one polynucleotide comprises any one of SEQ ID NOs: 1-16 or 134-164, in body fluids and kits for such purposes.

In addition to plasma or serum, over-representation or under-representation of at least one polynucleotide (at least one of SEQ ID NOs: 1-16 or 134-164) relative to an internal reference region may be determined in other body fluids isolated from a bovine animal including: whole blood, a product derived from blood, or any fraction derived from blood (in addition to plasma or serum.

Any known method may be used for the determination of over-representation or under-representation of at least one polynucleotide (at least one of SEQ ID NOs: 1-16 or 134-164) relative to an internal reference region in body fluids. Methods encompassed for such determinations include: polymerase chain reaction (PCR) amplification with sequence specific primer pairs, a hybridization-based method, enzyme-linked immunosorbent assay (ELISA), radioimmunoassay (RIA), solid-phase enzyme immunoassay (EIA), mass spectrometry, microarray analysis, and any combination thereof. Such methods when used for determining risk for developing JD or predicting onset of JD are encompassed herein.

In a particular method for determining over-representation or under-representation of any one of SEQ ID NOs: 1-16 or 134-164 relative to an internal reference region in cattle body fluids, e.g. serum, the method calls for PCR amplification with sequence specific primer pairs. In an embodiment thereof, the PCR amplification is performed with at least one primer pair specific for any one of SEQ ID NOs: 1-16 or 134-164. Exemplary primer pairs for amplifying any one of SEQ ID NOs: 1-16 are presented in Table 1. Conditions for performing PCR amplifications are known in the art and presented in the Examples herein below. Such conditions may be determined based in part on the composition of a primer and/or primer pair and considerations relating to same are known in the art.

In another particular method for determining over-representation or under-representation of any one of SEQ ID NOs: 1-16 or 134-164 relative to an internal reference region in cattle body fluids, e.g. serum, the method calls for an ELISA. In one embodiment, the ELISA for at least one of SEQ ID NOs: 1-16 or 134-164 involves a sandwich array. In such an embodiment, PCR amplification of at least one of SEQ ID NOs: 1-16 or 134-164 may be performed as an initial step. Conventional microtiter plates may be coated with a first antibody, e.g. a guinea pig polyclonal antibody, directed against any one of SEQ ID NOs: 1-16 or 134-164. The plates are then blocked and the sample or standard is loaded. After incubation with, e.g., at least one of SEQ ID NOs: 1-16 or 134-164, a second antibody against any one of SEQ ID NOs: 1-16 or 134-164 is applied, e.g. a polyclonal rabbit antibody. A third antibody that detects the second antibody, e.g. an anti-rabbit antibody, conjugated to a suitable label, e.g. an enzyme for chromogenic detection, is then added. The plate is then developed with a substrate for the label in order to detect and quantify the label, which in turn serves as a measure of any one of SEQ ID NOs: 1-16 or 134-164 in the body fluid. This determination may then be compared to that of an internal reference region measured by similar methodology. If the label is an enzyme for chromogenic detection, the substrate is a color-generating substrate of the conjugated enzyme and the color reaction is subsequently detected in a microplate reader and compared to standards.

Suitable pairs of antibodies that may be used as first and second antibodies are any combination of, e.g., guinea pig, rat, mouse, rabbit, goat, chicken, donkey or horse antibodies. In a particular embodiment, the antibodies are polyclonal antibodies. In another particular embodiment, the antibodies are monoclonal antibodies or antibody fragments. Suitable labels include: chromogenic labels (enzymes that can be used to convert a substrate to a detectable colored or fluorescent compound), spectroscopic labels (e.g., fluorescent labels), and affinity labels which may be developed by an additional compound specific for the label, thereby facilitating detection and quantification, or any other label used in standard ELISA.

Other preferred methods for detection of any one of SEQ ID NOs: 1-16 or 134-164 include radioimmunoassay or competitive immunoassay using a single antibody and chemiluminescence detection on automated commercial analytical robots. Microparticle enhanced fluorescence, fluorescence polarized methodologies, or mass spectrometry may also be used. Detection devices, e.g. microarrays, are also useful components as readout systems for any one of SEQ ID NOs: 1-16 or 134-164.

Also encompassed herein are kits for assessing over-representation or under-representation of any one of SEQ ID NOs: 1-16 or 134-164 relative to an internal reference region for determining risk for developing JD, which kits may comprise apparatus and reagents for detecting at least one of SEQ ID NOs: 1-16 or 134-164. Apparatus and reagents considered for PCR amplification include: suitable PCR primer pairs specific for each of SEQ ID NOs: 1-16 or 134-164, amplification reagents, and thermocycling devices. With respect to ELISA, microtiter plates for ELISA, pre-coated ELISA plates, and plate covers are encompassed. Reagents useful for ELISA include those antibodies and solutions developed and designed for detecting each of SEQ ID NOs: 1-16 or 134-164. Standard solutions comprising each of SEQ ID NOs: 1-16 or 134-164 as positive controls may be included in such kits. Kits may further comprise hardware, such as pipettes, solutions such as buffers, blocking solutions and the like, filters, and directions for use thereof.

The following definitions are presented as an aid to understand the invention.

The term “DNA” means a polymer composed of deoxyriboucleotides.

The terms “sample”, “biological sample”, “diagnostic sample”, and the like refer to a material known or suspected of expressing or containing one or more polynucleotide or polypeptide markers. The diagnostic sample may be any tissue ((e.g., blood, and fractions thereof, including serum, etc.).

The terms “polynucleotide” and “nucleic acid”, used interchangeably herein, describe a polymer of any length, e.g., greater than about 10 bases, greater than about 100 bases, greater than about 500 bases, greater than 1000 bases, usually up to about 10,000 or more bases composed of nucleotides, such as deoxyribonucleotides or ribonucleotides, or compounds produced synthetically which can hybridize with naturally occurring nucleic acids in a sequence specific manner analogous to that of two naturally occurring nucleic acids in Watson-Crick base pairing interactions. Polynucleotide and nucleic acid include polynucleotides that encode a native-sequence polypeptide, a polypeptide variant, a portion of a polypeptide, a chimeric polypeptide, or an isoform, precursor, complex, modified form, or derivative of any of the foregoing, and any precursors thereof. Polynucleotides can be deoxyribonucleotides, ribonucleotides, modified nucleotides or bases, and/or their analogs, or any substrate that can be incorporated into a polymer by DNA or RNA polymerase or by a synthetic reaction. A polynucleotide may be modified after synthesis (e.g., by conjugation with a label, such as a radioactive, chemiluminescent, chemiflourescent, or fluorescent label, and the like). Other types of modifications to polynucleotides known to a person skilled in the art include substitution of one or more naturally-occurring nucleotides with an analog, internucleotide modifications (e.g., uncharged linkages, charged linkages), and the like.

Polynucleotides can also include circulating nucleic acids (“CNA”). The term “circulating nucleic acid” or “CNA” refers to free nucleic acid, including RNA and DNA, circulating in the blood. CNA can include gene transcripts or other polynucleotide sequences. CNA can be obtained from any applicable biological sample, including blood, plasma, serum, and the like.

“Variants” of the sequences described herein are sequences wherein at least one nucleotide differs from that of the native or wild-type sequence (or the complement thereof), by virtue of an insertion, deletion, modification and/or substitution of one or more nucleotides within the native sequence. Such variants generally have less than 100% sequence identity relative to a native sequence or its complement. Accordingly, a sequence variant may have a nucleotide sequence with at least about 70%, 75%, 80%, 85%, 90%, 95%, 96%, 97%, 98%, or 99% sequence identity relative to the native or wild-type sequence or complement thereof. Variants, furthermore, may include fragments of any length that retain a biological activity of the corresponding native sequence. Variants also include sequences wherein one or more nucleotides are added to the 5′ or 3′ end of, or within, a native sequence or its complement.

“Percent sequence identity” is defined herein as the percentage of nucleotides or in the candidate sequence that are identical to the nucleotides in the sequence of interest after aligning the sequences and introducing gaps, if necessary, to achieve the maximum percent sequence identity, and not considering any conservative substitutions as part of the sequence identity. Methods and computer programs for the alignment of sequences are well known in the art, including, for example, “BLAST” algorithms.

“Oligonucleotides” include short, single-stranded polynucleotides that are at least seven nucleotides in length and less than about 250 nucleotides in length. The term “polynucleotides” includes oligonucleotides.

“Label” refers to a detectable compound or composition and “labelling” refers to the conjugation, fusion, or attachment of a detectable compound or composition to another. In some aspects described herein, the label is conjugated or fused directly or indirectly to a reagent, such as a polynucleotide probe or an antibody, and assists with the detection of the reagent to which it is conjugated or fused. The label itself can also be detectable (such as radioisotope labels or fluorescent labels and the like). In some aspects described herein, the label is an enzymatic label which catalyzes chemical alteration of a substrate compound or composition and results in a detectable product.

The term “diagnosis”, as used herein, refers to the identification or classification of a molecular or pathological state, disease, or condition (e.g., JD).). In a particular embodiment, JD is diagnosed in a subject (e.g., a bovine animal) in advance of onset of JD symptoms.

In a particular embodiment, a bovine subject is characterized as being “at risk for developing JD” because they have been exposed to or are suspected of having been exposed to MAP. Such exposures may be conveyed via exposure to a MAP-infected animal or an environmental feature (e.g., a field, water source, or food source) contaminated by bodily excretions/secretions (e.g., feces) of MAP-infected animals. Bovine subjects at risk for developing JD also include: very young cattle (e.g., less than one year old). Assays to evaluate risk for developing JD may also be implemented on all animals in a herd as a matter of routine practice. Such assays may also be performed in advance of acquisition of a new bovine animal so as to determine if the animal is a healthy, non-MAP-infected animal or an asymptomatic MAP-infected vector for JD transmission.

“Primer” refers to a polynucleotide capable of acting as a point of initiation of synthesis along a complementary strand when conditions are suitable for synthesis of a primer extension product. The synthesizing conditions include the presence of four different nucleotide bases (adenosine, cytidine, guanosine, thymidine/uridine) and at least one polymerization-inducing agent such as a reverse transcriptase or a DNA polymerase. The primers are present in a suitable buffer, which may include constituents which are co-factors or affect conditions such as pH and the like at various suitable temperatures. Primer includes single-stranded polynucleotide that is capable of hybridizing to nucleic acid and allowing the polymerization of a complementary nucleic acid, generally by providing a free 3′-OH group. Double stranded sequences can also be utilized. Primers are typically at least about 15 nucleotides. In some embodiments, primers can have a length of from about 15 to about 30, about 15 to about 50, about 15 to about 75, about 15 to about 100, or about 15 to about 500 nucleotides.

Exemplary primer pairs specific for each of SEQ ID NOs: 1-56 are presented herein in Table 1. Such primer pairs are selected based on their specificity for a particular polynucleotide and may be optimized for use in connection with, e.g., PCR amplification. Polynucleotide-specific primer pairs may comprise primers that include variations within their sequence such that the primer is no longer 100% complementary to the polynucleotide for which it is specific. Primers comprising such variations are encompassed herein as long as the variations do not alter the ability of the primer to amplify the polynucleotide with specificity. Such variations may also include nucleotides and/or tags at the 5′ and 3′ ends of the primer that are not complementary the polynucleotide for which the primer is specific. It is also understood that a primer or primer pair may be complementary to sequences that flank any one of SEQ ID NOs: 1-56 and SEQ ID NOs: 134-164 in the bovine genome and thus, may be used to amplify one of SEQ ID NOs: 1-56 and SEQ ID NOs: 134-164 in keeping with methods described herein. Design of such primers and primer pairs is well within the capabilities of one of ordinary skill in the art having read the present specification.

A “motif” or “sequence motif” refers to a nucleotide sequence pattern that is generally conserved across multiple species. Polynucleotides can be derived from the motif. The polynucleotides can correspond to the entire sequence of the motif or a portion or portions of the motif.

“Marker” or “biomarker” refers to an indicator which can be detected in a sample, and includes predictive, diagnostic, and prognostic indicators and the like. The marker can be an indicator of a particular disease or disorder (e.g., JD) having certain molecular, pathological, histological, and/or clinical features. Exemplary biomarkers include, without limitation, polynucleotides, polypeptides, polypeptide and polynucleotide modifications (such as post-translational modifications and the like), carbohydrates, and/or glycolipid-based molecular markers. The “presence”, “amount”, or “level” of a marker associated with an increased clinical benefit to an individual is a detectable level of the marker in a sample. The presence, amount, or level of a marker can be measured by methods known to a person skilled in the art. The presence, amount, or level of a marker may be measured prior to treatment, during treatment, after treatment, or a combination of any of the foregoing.

“Internal reference region” refers to a nucleic acid fragment circulating in a bodily fluid (e.g., blood or a fraction thereof such as serum) that is present in the same amount in both control subjects (those subjects who are not infected by MAP, those subjects who do not have JD, and/or those subjects who are not at risk for developing JD) and subjects who are at risk for developing JD or who have JD, respectively, as determined by RT-PCR experiments. Internal reference regions provide a nucleic acid fragment which is represented in the bodily fluid at a particular level, against which representation of other nucleic acid sequences (e.g., any one of SEQ ID NOs: 1-16 or 134-164), which differ in control subjects and MAP-infected subjects and are therefore discriminatory, can be evaluated in a relative manner.

In one embodiment, “over-representation” refers to a fold increase (relative quantity RQ) relative to at least 2^{-delta-delta Cq} of 2.

In one embodiment, “under-representation” refers to a fold decrease (relative quantity RQ) relative to minor as 2^{-delta-delta Cq} of 0,5.

The ΔCq method normalizes disease-specific motifs within a sample by subtracting the Cq value of the internal reference region (reference motif) from the Cq value of the disease specific motifs.

The 2-ΔΔCq method calculates relative quantity (RQ) of normalized disease specific motifs between two sample types (i.e. healthy and diseased). Information from multiple reference motifs can be combined to improve accuracy.

As used herein, the term “internal standard region” may refer to a composite of at least two individual internal standard regions. Accordingly, internal standard region may refer to a composite of at least 2, 3, 4, 5, 6, 7, 8, 9, 10, 11, 12, 13, 14, 15, 16, 17, 18, 19, 20, 25, 30, 35, 40, 45, or 50 or more individual internal standard regions. Exemplary internal reference regions are presented in Table 1 (SEQ ID NOs: 17-56) and over-representation and under-representation may be determined relative to the level of at least one of the internal reference regions.

Further to the above, in an embodiment, detection of over-representation or under-representation of at least one polynucleotide relative to an internal reference region in a biological sample or body fluid sample (e.g., serum), wherein the at least one polynucleotide comprises any one of, e.g., SEQ ID NOs: 1-16, is considered as a positive indicator that a bovine subject from which the sample was isolated will develop JD. Over-representation may be used to refer to a fold increase (relative quantity RQ) calculated as 2-delta-delta Cq value of at least 2, whereas under-representation refers to a fold decrease (relative quantity RQ) calculated as 2-delta-delta Cq value of at most 0.5.

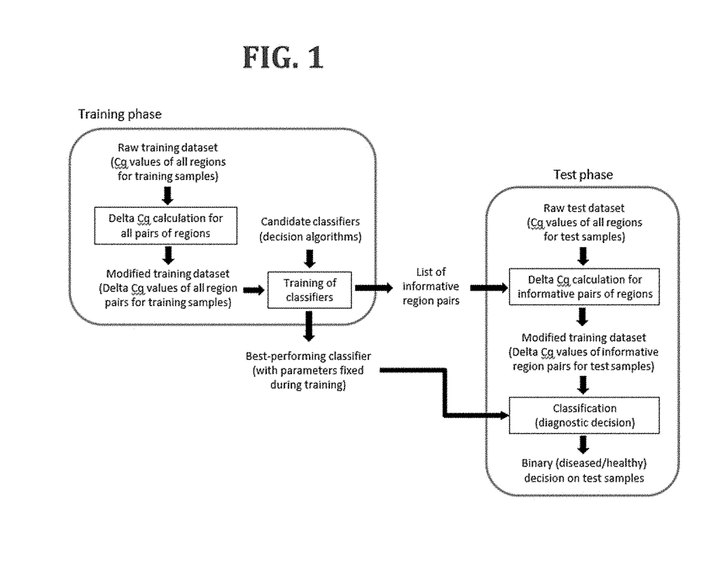

In another embodiment, for a given sample, the inventors use the Cq values for all (i.e. both old and new) regions (sequences) in the decision algorithm, which calculates the differences in Cq values (delta Cq) for certain informative pairs of regions and performs mathematical calculations on these delta Cq values. Based on these delta Cq values, the decision algorithm determines whether the collection of the delta Cq values as a whole is a positive indicator of Johne's disease. The informative pairs of regions to use, the specific decision algorithm to use, and the internal parameters of the decision algorithm are pre-determined based on a training dataset, but are fixed before the decision algorithm is used in a test setting. The decision algorithm is a binary (two-way, yes/no) classifier that assigns a class label (either diseased or healthy) to an input sample, thus determining the diagnosis. See FIG. 1 for flowchart illustrating the decision algorithm.

A specific set of decision algorithms (classifiers) are used with all sequences to determine the most informative region pairs and the final decision algorithm itself which together provide the classification performance on the training dataset. The current set of candidate classifiers comprises, without limitation, neural network, model averaged neural network, conditional inference random forest, distance weighted discrimination, and recursive partitioning. Sequences are then categorized as over-represented, under-represented, or internal reference based on the above classification scheme.

FIG. 1 presents an algorithm workflow illustrating a training phase and test phase of the algorithm whereby a determination of diseased versus healthy may be made in accordance with methods presented herein.

“Encode” refers to a polynucleotide “encoding” a polypeptide if, in its native state or when manipulated by methods well known to those skilled in the art, it can be transcribed and/or translated to produce the mRNA for the polypeptide and/or the polypeptide (or a fragment thereof). The anti-sense strand is the complement of such a nucleic acid, and the encoding sequence can be deduced therefrom.

“Array” or “microarray” refers to an ordered arrangement of hybridizable array elements on a substrate, such as solid substrate (e.g., glass slide and the like) or a semi-solid substrate (e.g., nitrocellulose membrane and the like). In some embodiments, the array elements may be polynucleotide probes (e.g. oligonucleotide). Array may include DNA microarrays (including cDNA microarrays, oligonucleotide microarrays, SNP microarrays, etc.), protein microarrays, peptide microarrays, antibody microarrays, and the like.

“Amplification” or “amplifying” refers to the production of one or more copies of a reference nucleic acid sequence or its complement. Amplification may be linear or exponential (e.g., in a polymerase chain reaction (PCR)). A nucleic acid copy produced from amplification may not have perfect sequence complementarity or identity relative to the reference sequence. In some embodiments, the copies can include nucleotide analogs, including deoxyinosine, intentional sequence alterations (such as alterations introduced through a primer that is hybridizable, but not fully complementary, to the template), and/or sequence errors that occur during the amplification process.

The terms “expression” and “expression level”, in general, are used interchangeably and generally refer to the amount of a marker in a sample. “Expression” generally refers to the process by which information (e.g., gene-encoded and/or epigenetic) is converted into the structures present and operating in the cell. Therefore, as used herein, “expression” can refer to transcription into a polynucleotide (such as mRNA and the like), translation into a polypeptide, or even polynucleotide and/or polypeptide modifications (e.g., post-translational modification of a polypeptide and the like). Fragments of the transcribed polynucleotide, the translated polypeptide, or polynucleotide and/or polypeptide modifications (such as post-translational modification of a polypeptide and the like) will also be regarded as expressed whether they originate from a transcript generated by alternative splicing or a degraded transcript, or from a post-translational processing of the polypeptide (e.g., by proteolysis). “Expressed genes” include those that are transcribed into a polynucleotide as mRNA and then translated into a polypeptide, and also those that are transcribed into RNA but not translated into a polypeptide (such as transfer and ribosomal RNAs and the like).

An “isolated” nucleic acid refers to a nucleic acid molecule that has been separated from a component of its natural environment. An isolated nucleic acid includes a nucleic acid molecule contained in cells that ordinarily contain the nucleic acid molecule. The nucleic acid molecule may be present extrachromosomally or at a chromosomal location that is different from its natural location.

An “isolated” antibody is an antibody which has been separated from a component of its natural environment. In some aspects described herein, an antibody is purified to greater than 95% or 99% purity as determined by methods known to a person skilled in the art, such as electrophoretic methods (e.g., SDS-PAGE, isoelectric focusing, capillary electrophoresis), chromatographic methods (e.g., ion exchange chromatography or reverse phase HPLC), and/or the like.

The term “sequencing” and its variants include obtaining sequence information from a strand of a nucleic acid molecule, typically by determining the identity of at least some nucleotides (including their nucleobase components) within the nucleic acid molecule. The term sequencing may also refer to determining the order of nucleotides (base sequences) in a nucleic acid sample (e.g. DNA or RNA). Many techniques are available and known to a person skilled in the art, such as Sanger sequencing, high-throughput sequencing technologies (such as the GS FLX platform offered by Roche Applied Science, Penzberg, Germany, based on pyro sequencing, or Illumina sequencing platforms, as offered by Illumina Inc., 5200 Illumina Way, San Diego, Calif. 92122, USA), and the like. High-throughput sequencing technologies refer to sequencing technologies having increased throughput as compared to traditional Sanger- and capillary-electrophoresis-based approaches (e.g., with the ability to generate hundreds of thousands or millions of relatively small sequence reads at a time). These high-through-put sequencing technologies include, but are not limited to, sequencing by synthesis, sequencing by ligation, pyrosequencing, sequencing by hybridization, and/or the like.

As used herein, “reactive” means the agent has affinity for, binds to, or is directed against specific CNAs. As further used herein, an “agent” includes a protein, polypeptide, peptide, nucleic acid (including DNA or RNA), antibody, Fab fragment, F(ab′)2 fragment, molecule, compound, antibiotic, drug, and any combinations thereof. A Fab fragment is a univalent antigen-binding fragment of an antibody, which is produced by papain digestion. A F(ab′)2 fragment is a divalent antigen-binding fragment of an antibody, which is produced by pepsin digestion. By way of example, the agent of the present invention can be labeled with a detectable marker. Agents that are reactive with CNAs can be identified by contacting the CNA with an agent of interest and assessing the ability of the agent to bind to the CNA.

In one embodiment of the present invention, the agent reactive with a JD biomarker is an antibody. Antibodies for use herein can be labeled with a detectable marker. Labeling of an antibody can be accomplished using one of a variety of labeling techniques, including peroxidase, chemiluminescent labels known in the art, and radioactive labels known in the art. The detectable marker of the present invention can be, for example, a nonradioactive or fluorescent marker, such as biotin, fluorescein (FITC), acridine, cholesterol, or carboxy-X-rhodamine (ROX), which can be detected using fluorescence and other imaging techniques readily known in the art. Alternatively, the detectable marker can be a radioactive marker, including, for example, a radioisotope. The radioisotope can be any isotope that emits detectable radiation, such as ³⁵S, ³²P, or ³H. Radioactivity emitted by the radioisotope can be detected by techniques well known in the art. For example, gamma emission from the radioisotope can be detected using gamma imaging techniques, particularly scintigraphic imaging. By way of example, such an agent can be a high-affinity antibody labeled with a detectable marker.

Where the agent is an antibody reactive with a JD biomarker, a biological sample taken from a mammal (e.g., a bovine animal) can be purified by passage through an affinity column which contains the antibody having affinity to the JD biomarker as a ligand attached to a solid support, such as an insoluble organic polymer in the form of a bead, gel, or plate. The antibody attached to the solid support can be used in the form of a column. Examples of suitable solid supports include, without limitation, agarose, cellulose, dextran, polyacrylamide, polystyrene, sepharose, and other insoluble organic polymers. The antibody can be further attached to the solid support through a spacer molecule, if desired. Appropriate binding conditions (e.g., temperature, pH, and salt concentration) can be readily determined by the skilled artisan. By way of example, the antibody can be attached to a sepharose column, such as Sepharose 4B.

Alternatively, a diagnostic sample from a bovine animal can be assayed using hybridization analysis of nucleic acid extracted from the diagnostic sample taken from the cattle to determine the presence of a JD biomarker, such as a CNA. In this aspect of the invention, the hybridization analysis can be conducted using Northern blot analysis of mRNA. This method can also be conducted by performing a Southern blot analysis of DNA using at least one nucleic acid probe which hybridizes to CNAs (including amplified CNAs). The nucleic acid probes of the present invention can be prepared by a variety of techniques known to those skilled in the art, including, without limitation, the following: restriction enzyme digestion of nucleic acid; and automated synthesis of oligonucleotides having sequences which correspond to selected portions of the nucleotide sequence of the JD biomarker, using commercially-available oligonucleotide synthesizers.

The nucleic acid probes used herein can be DNA or RNA, and can vary in length from about 5-20 nucleotides or 10-20 nucleotides to the entire length of the nucleic acid encoding for a JD biomarker. In some embodiments, the nucleic acid probes are oligonucleotides. The nucleic acid used in the probes can be derived from mammalian polynucleotide sequence complementary to the JD biomarker. In addition, the nucleic acid probes of the present invention can be labeled with one or more detectable markers. Labeling of the nucleic acid probes can be accomplished using one of a number of methods known in the art (e.g., nick translation, end labeling, fill-in end labeling, polynucleotide kinase exchange reaction, random priming, SP6 polymerase (for riboprobe preparation)) along with one of a variety of labels (e.g., radioactive labels, such as ³⁵S, ³²P, or ³H, or nonradioactive labels, such as biotin, fluorescein (FITC), acridine, cholesterol, or carboxy-X-rhodamine (ROX)). In some embodiments, these nucleic acid probes are used in an array or microarray.

In an exemplary approach, disease specific CNA motifs may be detected without any prior amplification step by implementing a hybridization-based procedure for the detection of JD-indicator markers using DNA probes labeled fluorescently. The probes may be the specific counterparts to the CNA molecules of interest and thus, hybridize to the JD-indicator CNA markers. Successful hybridization events produce a shift in the duration of the fluorescent signal, which shift can be measured and used as a parameter for the determination of the JD status of an animal.

In addition, the present invention provides a method of determining whether a bovine animal is at risk for JD, has been infected by MAP, and/or has already developed JD. The method includes analyzing a biological sample of the bovine animal for the presence of at least one JD biomarker, and optionally, further recommending a corroborative test for JD if the at least one JD biomarker is present in the biological sample. In some embodiments, the corroborative test includes ELISA, immunohistochemistry, and Western Blot/immunoblot or a combination of more than one of any of the foregoing.

In a further aspect, the present invention provides a method of determining progression of JD in cattle. The method includes analyzing a biological sample of the bovine animal for the presence of more than one JD biomarkers. The detection of the presence of more than one JD biomarker may be indicative of JD progressing in the bovine animal.

In the methods described herein, the step of analyzing a diagnostic sample can include obtaining the sample from the cattle (e.g., serum); using the serum directly or a diluted sample thereof (serum diluted in, e.g., water); amplifying the isolated nucleic acid using primers that are specific for or capable of amplifying a sequence corresponding to a JD CNA biomarker; and sequencing the amplified nucleic acid. In a more particular embodiment, the analyzing may include a step of isolating nucleic acids from the sample prior to amplification. n some embodiments, the isolated nucleic acid includes genomic DNA, mRNA, and/or cDNA obtained from mRNA. In some embodiments, the step of determining the representation of the at least one JD CNA marker includes use of at least one of a PCR-based detection method and a hybridization-based method. In some embodiments, the step of determining the representation of the at least one JD CNA marker includes an immunohistochemical analysis. In some embodiments, an array or a microarray is used for identifying the JD biomarker.

The diagnostic sample can be assayed for expression of JD biomarkers in vitro or in vivo. In addition, the diagnostic sample can be assayed for expression of JD biomarkers using all of the various assays and methods of detection and quantification described above.

The discovery that certain CNAs constitute JD biomarkers provides compositions and methods for identifying cattle at risk for developing JD and presents the potential for commercial application in the form of a test for the diagnosis of JD (including, e.g., early stage JD) and kits including same. The development of such a test or kit would provide general screening procedures; these procedures could assist in the early detection and diagnosis of JD in bovine subjects. Accordingly, the present invention further provides a kit for use as an assay of JD, comprising at least one agent reactive with a JD biomarker. The agent can be any of those described above, and can be used in any of the above-described assays or methods for detecting JD biomarkers.

Oligonucleotides complementary to a JD CNA biomarker can be designed based on the nucleotide sequence of the particular JD biomarker. A nucleotide sequence complementary to the selected partial sequence of the JD CNA biomarker can, e.g., be chemically synthesized using one of a variety of techniques known to those skilled in the art, including, without limitation, automated synthesis of oligonucleotides having sequences which correspond to a partial sequence of the JD CNA biomarker nucleotide sequence, or a variation sequence thereof, using commercially-available oligonucleotide synthesizers.

The present invention also provides the use of an oligonucleotide capable of identifying at least one JD CNA biomarker to determine the representation of same in a bovine subject. The oligonucleotide can be labelled with a detectable marker, such as a radioactive marker, fluorescent marker, the like, or a combination of any of the foregoing.

As described herein, the present inventors have identified DNA molecules, which circulate in the bovine bloodstream, the over- and under-representation of which can be used to discriminate between healthy and diseased animals afflicted by Johne's disease (i.e. bovine paratuberculosis) using DNA detection methods, including RT-PCR.

Serum from healthy individuals (controls) and diseased animals was harvested and initially stored at −80 degrees Celsius. Total DNA was extracted from the serum samples using the High Pure Viral Nucleic Acid Kit (Roche Applied Science; Cat. No. 11858874001). The DNA was amplified using the GenomePlex Single Cell Whole Genome Amplification Kit (Sigma; Cat. No. WGA4-500RXN) and purified using the GenElute™ PCR Clean-Up Kit (Sigma, Cat. No. NA1020-1KT). High-throughput paired-end DNA sequencing was performed by the University of Calgary's DNA Sequencing facility using Seq-It or NextSeq 500 sequencer machines. The resulting sequence reads were mapped to the bovine genome. Using an in-house Bioinformatics pipeline, which was established on the high-performance Bioinformatics infrastructure at the Institute of Computational Biotechnology, the present inventors have identified DNA motifs, that are present at distinctively high or distinctively low read count numbers in diseased animals, when compared to controls, corresponding to motifs that are over-represented and under-represented in JD, respectively. The motifs identified are the result of the host response (i.e. the response of the animal's body) to the infection with Mycobacterium avium ssp. paratuberculosis. The DNA motifs thus identified were used as targets for the development of a real-time Polymerase-Chain Reaction (RT-PCR) assay. In addition to the motifs that are present at distinctively different levels in healthy and infected animals (markers: over- and under-represented CNAs), motifs that are present at highly similar levels in healthy and diseased animals (internal reference regions) were also identified. The identification of internal reference regions facilitates normalization and accurate evaluation of the detected quantities of over- and under-represented JD-indicative DNA motifs across different animal samples and RT-PCR reactions. The RT-PCR-evaluated motifs, which are used to discriminate between healthy and diseased animals in a statistically significant manner, may be assessed as individual markers of JD or in combination with other motifs as a group.

Another aspect of the invention is arrays comprising one of more polynucleotides of the disclosure, PCR primers and/or probes for amplifying and/or detecting polynucleotides of the inventions, and methods of detecting JD indicative CNAs comprising an array or PCR primers and/or probes according to the disclosure.

Arrays comprising one of more polynucleotides of the disclosure, PCR primers and/or probes for amplifying and/or detecting polynucleotides (CNAs) described herein, and methods for detecting risk for developing JD comprising an array or PCR primers and/or probes are encompassed herein.

One of more polynucleotide sequences of the disclosure can be incorporated onto a sequence array, such as a biochip, DNA chip, BiofireDX filmarray and other filmarrays, microarray, macroarray, and the like, for screening, e.g., serum separated from whole blood from bovine animals for JD risk. Alternatively, CNAs can be extracted from the sample for screening on the array. Arrays are generally solid supports upon which a collection of polynucleotides and/or primers and/or probes are placed at defined locations on the array, either by spotting, printing, or direct synthesis. The array can include probes corresponding to one or more of the polynucleotides described herein (e.g., at least one of SEQ ID NOs: 1-56 and SEQ ID NOs: 134-164 and/or primers and/or probes for amplifying and/or detecting one or more polynucleotides of SEQ ID NOs: 1-56 and SEQ ID NOs: 134-164).

The underlying principle of arrays is base pairing or hybridization i.e., A-T and G-C for DNA, and A-U and G-C for RNA. A sample from a mammal (e.g., a bovine animal) is allowed to hybridize with the polynucleotides and/or primers and/or probes on the array providing an expression profile/pattern of CNA. The CNA expression pattern of JD-indicator sequences can be used to determine if a bovine animal is at risk for developing JD. The array can be prepared by any method known in the art. In some embodiments, a microarray is prepared generally as disclosed in U.S. Pat. No. 7,655,397, the entirety of which is hereby incorporated by reference.

In some embodiments, the array comprises at least 4 polynucleotides selected from polynucleotides comprising or consisting of SEQ ID NOs: 1-16 or 134-164 or primers or probes specific for at least 4 of SEQ ID NOs: 1-16 or 134-164. In another embodiment, the array comprises at least 8 polynucleotides selected from the polynucleotides comprising or consisting of SEQ ID NOs: 1-16 or 134-164 or primers or probes specific for at least 8 of SEQ ID NOs: 1-16 or 134-164. In some embodiments, the array comprises up to 19 polynucleotides comprising or consisting of SEQ ID NOs: 1-16 or 134-164 or primers or probes specific for at least 19 of SEQ ID NOs: 1-16 or 134-164. Arrays described herein may further comprise at least one of SEQ ID NOs: 17-56, which are internal reference region sequences. The array generally includes many copies of the selected polynucleotides to facilitate detection. In some embodiments, the array comprises a million or more copies of each of the selected polynucleotides.

Probes for detecting polynucleotides described herein can be designed and prepared using conventional methods. Software for modeling and designing probes and primers, including determining hybridization and annealing conditions, for detecting a specific polynucleotide sequence are publicly available, and include for example those of Integrated DNA Technologies, LightCycler® Probe Design Software (Roche Applied Science), Primer3 (Simgene), and FastPCR (PrimerDigital). See also techniques described by Illumina (Illumina Inc., 5200 Illumina Way, San Diego, Calif. 92122, USA).

The array can include positive indicator for JD sequences and/or probes for detecting same and negative and/or positive control sequences and/or probes.

Polynucleotides described herein can be amplified and/or detected via PCR, including but not limited to real-time PCR, multiplex PCR, nested PCR, solid phase PCR, miniprimer PCR, and the like. Primers and probes for amplifying and/or detecting polynucleotides described herein can be designed and prepared using conventional methods. Software for modeling and designing primers and probes, including determining hybridization, melting, annealing, and/or extensions conditions, for amplifying and/or detecting a specific polynucleotide sequence are publicly available, and include for example LightCycler® Probe Design Software (Roche Applied Science), Primer3 (Simgene), and FastPCR (PrimerDigital). See also techniques described by Illumina (Illumina Inc., 5200 Illumina Way, San Diego, Calif. 92122, USA). PCR conditions generally include the presence of four different nucleotide bases (adenosine, cytidine, guanosine, thymidine/uridine) and at least one polymerization-inducing agent such as a reverse transcriptase or a DNA polymerase. The primers are generally present in a suitable buffer, which may include constituents, which are co-factors or affect conditions such as pH and the like at various suitable temperatures. The primers are preferably single-strand nucleotide sequences, such that amplification efficiency of the desired polynucleotide is optimized. Double-stranded nucleotide sequences can also be utilized. The primers are typically at least about 15 nucleotides. In some embodiments, the primers can have a length of from about 15 to about 30, about 15 to about 50, about 15 to about 75, about 15 to about 100, or about 15 to about 500 nucleotides.

In some embodiments, primer sets are designed to amplify one or more of the at risk for JD-indicative polynucleotides comprising or consisting of SEQ ID NOs: 1-16 or 134-164 and then the PCR products of the primer sets are screened for JD-indicator sequences on an array as described herein. Primer sets designed to amplify one or more of the at least one of SEQ ID NOs: 17-56, which are internal reference region sequences, are also encompassed.

Diagnostic kits comprising one or more primer pairs, and optional probes, for amplifying and detecting one or more polynucleotides described herein are also provided. The kit can optionally include nucleotide bases (adenosine, cytidine, guanosine, thymidine/uridine) and at least one polymerization-inducing agent such as a reverse transcriptase or a DNA polymerase. The kit can optionally include a suitable primer buffer, which may include constituents which are co-factors or affect conditions such as pH and the like at various suitable temperatures. The kit can optionally include an array as described herein.

The primers provided in the diagnostic kit are generally provided at least in pairs (forward primer and reverse primer) for amplifying a specific polynucleotide sequence. These primers can be used to amplify and detect CNAs in blood serum from cattle, or any other appropriate biological sample from cattle that may contain CNAs. Alternatively, CNAs can be extracted from the sample and then amplified by PCR using a diagnostic kit of the disclosure. The CNA expression pattern of JD-indicative sequences detected by the diagnostic kit can be used to determine if a bovine animal is at risk for developing JD even at a stage wherein no clinical symptoms of JD are apparent.

In some embodiments, the kit comprises primers for amplifying at least 4 polynucleotides selected from the polynucleotides comprising or consisting of SEQ ID NOs: 1-16 or 134-164, and optionally one or more probes for detecting the amplified product. In another embodiment, the kit comprises primers for amplifying at least 8 polynucleotides selected from the polynucleotides comprising or consisting of SEQ ID NOs: 1-16 or 134-164, and optionally one or more probes for detecting the amplified product. In some embodiments, the kit comprises primers for amplifying up to 16 polynucleotides comprising or consisting of SEQ ID NOs: 1-16 or 134-164, and optionally one or more probes for detecting the amplified product.

### Treatment of MAP-Infected Cattle

In that JD is caused by MAP, antimicrobial therapy is envisioned as a reasonable therapeutic regimen for treatment thereof. In a particular embodiment, the antimicrobial therapy comprises administration of antibiotics, which may be administered alone or in conjunction with anti-inflammatory therapy. The choice of antibiotic used for treating a bovine animal afflicted with JD is based on the drug susceptibility profiles of MAP clinical isolates. Macrolide drugs, such as, for example, clarithromycin and azithromycin exhibit the greatest in vitro efficacy. The rifampicin family of antibiotics also exhibits in vitro efficacy against MAP. Accordingly, in a particular embodiment, at least one of a macrolide drug (such as, e.g., clarithromycin, azithromycin, amikacin, ciprofloxacin, and levofloxacin) and an antibiotic in the rifampicin family (e.g., rifampicin, rifabutin, and ethambutol) is administered to a bovine animal afflicted with JD. An exemplary anti-inflammatory therapy that may be administered in conjunction with antibiotic therapy involves administration of non-steroidal anti-inflammatory agents to the bovine animal afflicted with JD.

In a particular embodiment, a bovine animal which is identified as MAP-infected using methods and/or reagents described herein is treated with 20 mg/kg of an antibiotic in the rifampicin family (e.g., rifampicin, rifabutin, and ethambutol) alone or in combination with 20 mg/kg of isoniazid. In a more particular embodiment, a bovine animal which is identified as MAP-infected using methods and/or reagents described herein is treated with 10-20 mg/kg of rifampicin alone or in combination with 20 mg/kg of isoniazid. In a still more particular embodiment, a bovine animal which is identified as MAP-infected using methods and/or reagents described herein is treated with 10-20 mg/kg of rifampin.

The ionophore antibiotic, monensin, also offers promise as a therapeutic agent for treating JD in adult cattle and calves. Monensin may also serve as a chemoprophylactic for calves. Monensin inhibits in vitro growth of certain MAP strains. Data from in vivo studies is, moreover, encouraging. Provision of monensin to adult cattle naturally infected with MAP has been associated with modest improvements in histopathology scores (i.e., less tissue damage resulting from infection), decline in fecal shedding rate, and/or reduced odds of testing positive on a milk ELISA. Monensin used as a chemoprophylactic in calf milk replacer in dairy calves resulted in reduced tissue colonization and MAP fecal shedding.

Accordingly, monensin may be administered to a bovine animal which is identified as MAP-infected using methods and/or reagents described herein to reduce MAP infection levels. In a more particular embodiment, a bovine animal which is identified as MAP-infected using methods and/or reagents described herein is treated with Monensin CRC (Rumensin® CRC) administered in capsule form supplied by Elanco, a Division Eli Lilly Canada, Guelph, Ont., Canada. The monensin CRC is a sustained-release intraruminal device that has a medicated core containing 32 g monensin in a hexaglycerol distearate matrix (45% monensin). Each capsule delivers its product over an average period of 95 days (Rumensin CRC, Veterinary Reference Guide, Elanco Animal Health, A Division of Eli Lilly Canada Inc.). Capsules are administered orally with a specially designed administration tool. In another embodiment, monensin may be administered to a bovine animal which is identified as MAP-infected using methods and/or reagents described herein at a dose of 450 mg/head for 120 days.

In another embodiment, a bovine animal which is identified as MAP-infected using methods and/or reagents described herein is treated daily with 10-20 mg/kg isoniazid.

In another embodiment, a bovine animal which is identified as MAP-infected using methods and/or reagents described herein is treated daily with 2 mg/kg to 15 mg/kg of clofazimine administered orally. In yet another embodiment, a bovine animal which is identified as MAP-infected using methods and/or reagents described herein is treated daily with 600-1,000 mg of clofazimine administered orally.

In a further embodiment, a bovine animal which is identified as MAP-infected using methods and/or reagents described herein is treated daily with 10-20 mg/kg of rifampin administered orally.

In another embodiment, a bovine animal which is identified as MAP-infected using methods and/or reagents described herein is treated daily with 20 mg/kg of gallium nitrate.

In yet another embodiment, a bovine animal which is identified as MAP-infected using methods and/or reagents described herein is administered a once-daily oral treatment comprising rifampin (10-20 mg/kg) and isoniazid (10-20 mg/kg). In a more particular embodiment, monensin should be administered in conjunction with rifampin (10-20 mg/kg) and isoniazid (10-20 mg/kg) at a dose of 200 mg/head/day monensin for beef cattle or a dose of 410 mg/head/day monensin for dairy cattle

The aforementioned therapeutic regimens may be supplemented with levamisole as an adjunctive therapy. In a particular embodiment thereof, the levamisole is administered via weekly injections at a dosage of 2.5 mg/kg.

The present invention is described in the following Examples, which are set forth to aid in an understanding of the invention, and should not be construed to limit in any way the scope of the invention as defined in the claims which follow thereafter.

### EXAMPLES

### Example 1—Identification of Unique Johne's Disease-Associated Motifs in CNAS

The present inventors have identified CNAs, the over- and under-representation of which can be used to discriminate between healthy cattle and diseased cattle afflicted by Johne's disease (i.e. bovine paratuberculosis) using DNA detection methods, including RT-PCR.

Serum from healthy individuals (controls) and diseased animals was harvested and total DNA was extracted from the serum samples. The DNA was amplified and high-throughput paired-end DNA sequencing was performed. The resulting sequence reads were mapped to the bovine genome. The present inventors identified DNA motifs, which were present at different read count numbers in MAP-infected animals when compared to controls. The motifs identified reflect the host response to the infection with Mycobacterium avium ssp. paratuberculosis. The DNA motifs identified were used as targets for the development of a RT-PCR assay. In addition to the motifs, which are present at different levels in healthy and infected animals (markers; over- and under-represented CNAs), motifs which were present at the same level in healthy and infected animals (internal reference regions) were also identified. The identification of internal reference regions facilitates normalization of the results. The RT-PCR-evaluated motifs, which are used to discriminate between healthy and infected animals in a statistically significant manner, may be assessed as individual markers of JD or in combination with each other.

1 bov-jd-0001: 25:32377622-32377759; Over-Represented

1.1 Sequence

(SEQ ID NO: 1)

TACCATGGTGACCACGGGTGACGGGGAATCAGGGTTCGATTCCGGAGAGG
GAGCCTGAGAAACGGCTACCACATCCAAGGAAGGCAGCAGGCGCGCAAAT
TACCCACTCCCGACCCGGGGAGGTAGTGACGAAAAATA

1.2 Sequence with 25 bp Surroundings

(SEQ ID NO: 2)

AACTTTCGATGGTAGTCGCTGTGCCTACCATGGTGACCACGGGTGACGGG
GAATCAGGGTTCGATTCCGGAGAGGGAGCCTGAGAAACGGCTACCACATC
CAAGGAAGGCAGCAGGCGCGCAAATTACCCACTCCCGACCCGGGGAGGTA
GTGACGAAAAATAACAATACAGGACTCTTTGAGGCC

1.3 Annotation

1.3.1 Genes

1.3.2 Next Upstream Gene

### RefSeqID: NM_001192067

Gene name: GALNT17
Product: putative polypeptide N-acetylgalactosaminyltransferase-like protein 3
Translation start: 29391799

### Translation end: 29824281 Distance to

region end: 2553340 Exons: 11

1.3.3 Next Downstream Gene

### RefSeqID: NR_046257

Gene name: RN45S Product:
Translation start: 32380939

### Translation end: 32469510 Distance to

region end: 3181 Exons: 9

1.3.4 Repeats

Repeat name: SSU-rRNA_Hsa Repeat
class: rRNA
Repeat family: rRNA Repeat start:
32377234

### Repeat end: 32377936

2 bov-jd-0002: 18:58238154-58238329; Over-Represented

2.1 Sequence

(SEQ ID NO: 3)

GCTATCCTGAGGGAAACTTCGGAGGGAACCAGCTACTAGATGGTTCGATT
AGTCTTTCGCCCCTATACCCAGGTCGGACGACCGATTTGCACGTCAGGAC
CGCTACGGACCTCCACCAGAGTTTCCTCTGGCTTCGCCCTGCCCAGGCAT
AGTTCACCATCTTTCGGGTCCTAACA

2.2 Sequence with 25 bp Surroundings

(SEQ ID NO: 4)

GGTCTGTGCGTTTGCGAGAGCGCCAGCTATCCTGAGGGAAACTTCGGAGG
GAACCAGCTACTAGATGGTTCGATTAGTCTTTCGCCCCTATACCCAGGTC
GGACGACCGATTTGCACGTCAGGACCGCTACGGACCTCCACCAGAGTTTC
CTCTGGCTTCGCCCTGCCCAGGCATAGTTCACCATCTTTCGGGTCCTAAC
ACGTGCGCTCATGCTCCACCTCCCC

2.3 Annotation

2.3.1 Genes

2.3.2 Next Upstream Gene

### RefSeqID: NM_001075708

Gene name: ZNF350
Product: zinc finger protein 350
Translation start: 58212121

### Translation end: 58220105 Distance to

region end: 18048 Exons: 5

2.3.3 Next Downstream Gene

### RefSeqID: NM_001037477

Gene name: PPP2R1A
Product: serine/threonine-protein phosphatase 2A 65 kDa regulatory subunit A alpha isoform
Translation start: 58425841

### Translation end: 58448792 Distance to

region end: 187513 Exons: 15

2.3.4 Repeats

Repeat name: LSU-rRNA_Hsa Repeat
class: rRNA
Repeat family: rRNA Repeat start:
58238080

### Repeat end: 58238908

3 bov-jd-0003: X:62078561-62078748; Over-Represented

3.1 Sequence

(SEQ ID NO: 5)

TTCGCTGGATAGTAGGTAGGGACAGTGGGAATCTCGTTCATCCATTCATG
CGCGTCACTAATTAGATGACGAGGCATTTGGCTACCTTAAGAGAGTCATA
GTTACTCCCGCCGTTTACCCGCGCTTCATTGAATTTCTTCACTTTGACAT
TCAGAGCACTGGGCAGAAATCACATCGCGTCAACACCC

3.2 Sequence with 25 bp Surroundings

(SEQ ID NO: 6)

CCAAGCCCGTTCCCTTGGCTGTGGTTTCGCTGGATAGTAGGTAGGGACAG
TGGGAATCTCGTTCATCCATTCATGCGCGTCACTAATTAGATGACGAGGC
ATTTGGCTACCTTAAGAGAGTCATAGTTACTCCCGCCGTTTACCCGCGCT
TCATTGAATTTCTTCACTTTGACATTCAGAGCACTGGGCAGAAATCACAT
CGCGTCAACACCCGCCGCGGGCCTTCGCGATGCTTTG

3.3 Annotation

3.3.1 Genes

3.3.2 Next Upstream Gene

### RefSeqID: NM_001001171

Gene name: ATG4A
Product: cysteine protease ATG4A Translation
start: 61017761

### Translation end: 61050945 Distance to

region end: 1027615 Exons: 12

3.3.3 Next Downstream Gene

### RefSeqID: NM_174549

Gene name: GUCY2F
Product: retinal guanylyl cyclase 2 precursor
Translation start: 62214455

### Translation end: 62364957 Distance to

region end: 135708 Exons: 20

3.3.4 Repeats

Repeat name: LSU-rRNA_Hsa Repeat
class: rRNA
Repeat family: rRNA
Repeat start: 62078135

### Repeat end: 62078930

4 bov-jd-0004: 21:2014523-2014688; Over-Represented

4.1 Sequence

(SEQ ID NO: 7)

AGATGGATTTAGAGTGTGTGGCTGTTGTGTGTGGTTCAGAATTCTGAGTA
GTCCTTTCAGGGATCACCCAAAAGCTCAGACAAAGCGTGAGCCCTTCACG
TGGACTGTGCTGTGCGTCAGAAAGATAATACGAGCCACCTACACAATCTA
AAATTTCCTAAAAACC

4.2 Sequence with 25 bp Surroundings

(SEQ ID NO: 8)

TGAACACTGAATGTGTTTGCATTATAGATGGATTTAGAGTGTGTGGCTGT
TGTGTGTGGTTCAGAATTCTGAGTAGTCCTTTCAGGGATCACCCAAAAGC
TCAGACAAAGCGTGAGCCCTTCACGTGGACTGTGCTGTGCGTCAGAAAGA
TAATACGAGCCACCTACACAATCTAAAATTTCCTAAAAACCAACGGGCTA
CTCGACGTCCTCCTT

4.3 Annotation

4.3.1 Genes

4.3.2 Next Upstream Gene

### RefSeqID: NM_001014982

Gene name: NDN Product:
necdin Translation start: 736182

### Translation end: 737792 Distance to

region end: 1276730 Exons: 1

4.3.3 Next Downstream Gene

### RefSeqID: NM_001098462

Gene name: UBE3A
Product: ubiquitin-protein ligase E3A Translation
start: 2346808

### Translation end: 2410193 Distance to

region end: 332121 Exons: 12

4.3.4 Repeats

Repeat name: MER33 Repeat
class: DNA
Repeat family: hAT-Charlie Repeat start:
2014704

### Repeat end: 2014825

5 bov-jd-0005: 1:33090510-33090673; Over-Represented

5.1 Sequence

(SEQ ID NO: 9)

AGCTGAAGTGGTGGGATAGTGTAGTTTGTGTGTCATAGGAATCTCCTCAA
AGGATGATGTCATCAGTGTGAGGTCCTAATTGTGTCCTAGAAAAAGTTAG
AGCTGGTGAAAATGTTGCCTTCAAAGACTGTGTGAGATGGCAGGTGTGGA
CACTGGCCCCACTG

5.2 Sequence with 25 bp Surroundings

(SEQ ID NO: 10)

TTTACCAAAAAGTTTTCAGGAGCTTAGCTGAAGTGGTGGGATAGTGTAGT
TTGTGTGTCATAGGAATCTCCTCAAAGGATGATGTCATCAGTGTGAGGTC
CTAATTGTGTCCTAGAAAAAGTTAGAGCTGGTGAAAATGTTGCCTTCAAA
GACTGTGTGAGATGGCAGGTGTGGACACTGGCCCCACTGAGTAGACAGGT
GACGGTGTATCGT

5.3 Annotation

5.3.1 Genes

5.3.2 Next Upstream Gene

### RefSeqID: NM_001122729

Gene name: GBE1
Product: 1,4-alpha-glucan-branching enzyme
Translation start: 28659455

### Translation end: 28972472 Distance to

region end: 4118037 Exons: 15

5.3.3 Next Downstream Gene

### RefSeqID: NM_001191130

Gene name: VGLL3
Product: transcription cofactor vestigial-like protein 3
Translation start: 34657637

### Translation end: 34707554 Distance to

region end: 1566965 Exons: 4

5.3.4 Repeats

Repeat name: HERVL74-int Repeat class:

### LTR

Repeat family: ERVL Repeat start:
33090536

### Repeat end: 33090930

6 bov-jd-0006: 6:5440642-5440791; Over-Represented

6.1 Sequence

(SEQ ID NO: 11)

GGGTGTTGGTTCTCCTTGCAGGTCCTGGAGATACAGCCAAAGGCCCAGTT
TTTGAGAGAACTCTTCCTAACTGTCTTCTTGTCTGAAACTAATGTAGAAG
AGAGATTTTCACTAGTAGTAGTTTAGCTTATTTAGCCTGATTTCC ACCA
C

6.2 Sequence with 25 bp Surroundings

(SEQ ID NO: 12)

TTTTGGCTTTGTGGGTGGTAGACATGGGTGTTGGTTCTCCTTGCAGGTCC
TGGAGATACAGCCAAAGGCCCAGTTTTTGAGAGAACTCTTCCTAACTGTC
TTCTTGTCTGAAACTAATGTAGAAGAGAGATTTTCACTAGTAGTAGTTTA
GCTTATTTAGCCTGATTTCCACCACTTACATTCAGCAGCATGTTCATGG

6.3 Annotation

6.3.1 Genes

6.3.2 Next Upstream Gene

### RefSeqID: NM_001192907

Gene name: PRDM5
Product: PR domain zinc finger protein 5
Translation start: 4491174

### Translation end: 4743136 Distance to

region end: 697505 Exons: 16

6.3.3 Next Downstream Gene

### RefSeqID: NM_001079796

Gene name: LOC780876
Product: MAD2 mitotic arrest deficient-like 1 (yeast)-like
Translation start: 5536134

### Translation end: 5566460 Distance to

region end: 95344 Exons: 5

6.3.4 Repeats

7 bov-jd-0007: 14:1093166-1093354; Over-Represented

7.1 Sequence

(SEQ ID NO: 13)

GTTGTTTGTAATATTACTTAGGTATTTTTAACATTTTTATTGGAAAGTGA
AAGTTAGTTTTGGTTGTGCTGGGTTTTCATTGCTGTGTGCAGTCTTTCCC
TAGTTGCAGAGTGTGGGGGCTACTCTTTGTTTTAGGCTTTTCATTGTGGT
GGCTTCTCTTGTTGCAGAGCCTGGGCTGCAGAGTGTGTG

7.2 Sequence with 25 bp Surroundings

(SEQ ID NO: 14)

AAATTGTCTGGTTTATATGCATAGAGTTGTTTGTAATATTACTTAGGTAT
TTTTAACATTTTTATTGGAAAGTGAAAGTTAGTTTTGGTTGTGCTGGGTT
TTCATTGCTGTGTGCAGTCTTTCCCTAGTTGCAGAGTGTGGGGGCTACTC
TTTGTTTTAGGCTTTTCATTGTGGTGGCTTCTCTTGTTGCAGAGCCTGGG
CTGCAGAGTGTGTGGGCTTCAGTAGTTGTGGCACATGG

7.3 Annotation

7.3.1 Genes

7.3.2 Next Downstream Gene

### RefSeqID: NM_001046153

Gene name: C14H8orf33
Product: UPF0488 protein C8orf33 homolog
Translation start: 1487365

### Translation end: 1489409 Distance to

region end: 394012 Exons: 6

7.3.3 Repeats

Repeat name: L1MD Repeat
class: LINE Repeat family: L1
Repeat start: 1092897

### Repeat end: 1093218 Repeat

name: CHR-2A Repeat class:
SINE Repeat family: tRNA
Repeat start: 1093218

### Repeat end: 1093482

8 bov-jd-0008: 6:5425625-5425755; Under-Represented

8.1 Sequence

(SEQ ID NO: 15)

GATCTTATTCTTTGAAGTAATGTTATGAATCTTATCGGACAAAGCCAGAC
TTCTTCATTTGGGAGTAGATTTGGAAGAGAACACTTGGTCTCTCAGCACT
CCCCTTCTCAGGAAGGTTACAGAAACCAAAA

8.2 Sequence with 25 bp Surroundings

(SEQ ID NO: 16)

TTCTGACTCTGCAGTACCTAAATCAGATCTTATTCTTTGAAGTAATGTTA
TGAATCTTATCGGACAAAGCCAGACTTCTTCATTTGGGAGTAGATTTGGA
AGAGAACACTTGGTCTCTCAGCACTCCCCTTCTCAGGAAGGTTACAGAAA
CCAAAATAAGACCTACCAGAATTGGGGTGG

8.3 Annotation

8.3.1 Genes

8.3.2 Next Upstream Gene

### RefSeqID: NM_001192907

Gene name: PRDM5
Product: PR domain zinc finger protein 5
Translation start: 4491174

### Translation end: 4743136 Distance to

region end: 682488 Exons: 16

8.3.3 Next Downstream Gene

### RefSeqID: NM_001079796

Gene name: LOC780876
Product: MAD2 mitotic arrest deficient-like 1 (yeast)-like
Translation start: 5536134

### Translation end: 5566460 Distance to

region end: 110380 Exons: 5

8.3.4 Repeats

9 bov-jd-0009: 1:29318042-29318377; Internal Reference Region

9.1 Similarities

14 bov-jd-0021: 25-36122654-36123000: 96.0%

10 bov-jd-0010: 7-34812837-34813209: 83.6%

9.2 Sequence

(SEQ ID NO: 17)

GTTTGTAACCTGGGACCCTTGAGTTAATTCTTTTTCTTGTTATAGCCCAC
CACACCTTTGCTCTGTAGGAATGCAACTTTATCTAATGCTTTTTTGGAGG
GTGGCTCCTGACCAACCACCTTTAGAGAAAAATAAGTTTTCTGAAGAAAA
GGTCTTAAAATGTTAACAGGCCTCCGGGCCAGAAGATGATGCAAATCACC
TAAGCTTTTGCATATGATAAGTTTGCAGGAAGAAAGCCTGGTTTGCTGCA
AGACTCGACCCCTTCCCCCATTATCCTCTATGCATAACTTAAGGTATAAA
AACTACTTTGAAAAATAAAGTGCGGGCCTTGTTCAC

9.3 Sequence with 25 bp Surroundings

(SEQ ID NO: 18)

ATTGGTGTACCGTAGTAGCTTTAATGTTTGTAACCTGGGACCCTTGAGTT
AATTCTTTTTCTTGTTATAGCCCACCACACCTTTGCTCTGTAGGAATGCA
ACTTTATCTAATGCTTTTTTGGAGGGTGGCTCCTGACCAACCACCTTTAG
AGAAAAATAAGTTTTCTGAAGAAAAGGTCTTAAAATGTTAACAGGCCTCC
GGGCCAGAAGATGATGCAAATCACCTAAGCTTTTGCATATGATAAGTTTG
CAGGAAGAAAGCCTGGTTTGCTGCAAGACTCGACCCCTTCCCCCATTATC
CTCTATGCATAACTTAAGGTATAAAAACTACTTTGAAAAATAAAGTGCGG
GCCTTGTTCACCGAAACTTGGTCTCACCATGTCGT

9.4 Annotation

9.4.1 Genes

9.4.2 Next Upstream Gene

### RefSeqID: NM_001122729

Gene name: GBE1
Product: 1,4-alpha-glucan-branching enzyme
Translation start: 28659455

### Translation end: 28972472 Distance to

region end: 345569 Exons: 15

9.4.3 Next Downstream Gene

### RefSeqID: NM_001191130

Gene name: VGLL3
Product: transcription cofactor vestigial-like protein 3
Translation start: 34657637

### Translation end: 34707554 Distance to

region end: 5339261 Exons: 4

9.4.4 Repeats

Repeat name: BTLTR1 Repeat
class: LTR Repeat family:
ERVK Repeat start: 29317937

### Repeat end: 29318587

10 bov-jd-0010: 7:34812863-34813185; Internal Reference Region

10.1 Similarities

9 bov-jd-0009: 1-29318016-29318401: 83.6%

14 bov-jd-0021: 25-36122654-36123000: 73.7%

16 bov-jd-0023: 28-40139930-40140429: 75.2%

10.2 Sequence

(SEQ ID NO: 19)

GTGTCCCTGGCAGTGTATTGATTAATATAATTGGTGTAAGTAGTAGCTTT
AATGTTTGTAACCTGGGACCCTTGAGTTAATTCTTTTTCTTGTTATAGCC
CACCACACCTTTGCTCTGTAGGAATGCAACTTTATCTAATGCTTTTTTGG
AGGGTGGCTCCTGACCAACCACCTTTAGAGAAAAATAAGTTTTCTGAAGA
AAAGGTCTTAAAATGTTAACAGGCCTCCGGGCCAGAAGATGATGCAAATC
ACCTAAGCTTTTGCATATGATAAGTTTGCAGGAAGAAAGCCTGGTTTGCT
GCAAGACTCGACCCCTTCCCCCA

10.3 Sequence with 25 bp Surroundings

(SEQ ID NO: 20)

CTAAGTTTCCATATCCATTAGTGCTGTGTCCCTGGCAGTGTATTGATTAA
TATAATTGGTGTAAGTAGTAGCTTTAATGTTTGTAACCTGGGACCCTTGA
GTTAATTCTTTTTCTTGTTATAGCCCACCACACCTTTGCTCTGTAGGAAT
GCAACTTTATCTAATGCTTTTTTGGAGGGTGGCTCCTGACCAACCACCTT
TAGAGAAAAATAAGTTTTCTGAAGAAAAGGTCTTAAAATGTTAACAGGCC
TCCGGGCCAGAAGATGATGCAAATCACCTAAGCTTTTGCATATGATAAGT
TTGCAGGAAGAAAGCCTGGTTTGCTGCAAGACTCGACCCCTTCCCCCATT
ATCCTTCTATGCATAACTTAAG

10.4 Annotation

10.4.1 Genes

10.4.2 Next Upstream Gene

### RefSeqID: NM_001076190

Gene name: FTMT
Product: ferritin, mitochondrial precursor
Translation start: 33311976

### Translation end: 33313090 Distance to

region end: 1499772 Exons: 1

10.4.3 Next Downstream Gene

### RefSeqID: NM_001007809

Gene name: HSD17B4
Product: peroxisomal multifunctional enzyme type 2
Translation start: 35662598

### Translation end: 35763607 Distance to

region end: 849414 Exons: 24

10.4.4 Repeats

Repeat name: BTLTR1 Repeat
class: LTR Repeat family:
ERVK Repeat start: 34812348

### Repeat end: 34813462

11 bov-jd-0011: 3:66900607-66900863; Internal Reference Region

11.1 Similarities

12 bov-jd-0012: 4-12626463-12626916: 79.0%

11.2 Sequence

(SEQ ID NO: 21)

ACTACTTACACCAATTATATTAATCAATACACTGCCAGGGACACAGCAGG
TAAGGGATATGGAAACTTAGCAGCAAACAATGGCCCAACAAGTGAAAAAC
CATTCACCAATACAATTTCTAATCAATCTTGTAACTACTCAAAAGAATCT
GTGTTTAGACAGTTTAGAACATCTCCTGCCTCTCACAGTTGGGAGGCTCT
GAACAATCCCATGTGGCCGGAAAAACCTATTCAGGCAGGCTAGAGGATTT
CCAAAGG

11.3 Sequence with 25 bp Surroundings

(SEQ ID NO: 22)

GTCCCAGGTTACAAACATTAAAGCTACTACTTACACCAATTATATTAATC
AATACACTGCCAGGGACACAGCAGGTAAGGGATATGGAAACTTAGCAGCA
AACAATGGCCCAACAAGTGAAAAACCATTCACCAATACAATTTCTAATCA
ATCTTGTAACTACTCAAAAGAATCTGTGTTTAGACAGTTTAGAACATCTC
CTGCCTCTCACAGTTGGGAGGCTCTGAACAATCCCATGTGGCCGGAAAAA
CCTATTCAGGCAGGCTAGAGGATTTCCAAAGGAGTTTGTAGGTTAAACAC
TGTCAC

11.4 Annotation

11.4.1 Genes

11.4.2 Next Upstream Gene

### RefSeqID: NM_001080279

Gene name: GIPC2
Product: PDZ domain-containing protein GIPC2 Translation
start: 66795565

### Translation end: 66895377 Distance to

region end: 5229 Exons: 6

11.4.3 Next Downstream Gene

### RefSeqID: NM_001046503

Gene name: DNAJB4
Product: dnaJ homolog subfamily B member 4
Translation start: 66922935

### Translation end: 66931502 Distance to

region end: 22073 Exons: 3

11.4.4 Repeats

Repeat name: BTLTR1 Repeat
class: LTR Repeat family:
ERVK Repeat start: 66900561

### Repeat end: 66901198

12 bov-jd-0012: 4:12626489-12626892; Internal Reference Region

12.1 Similarities

11 bov-jd-0011: 3-66900581-66900887: 79.0%

17 bov-jd-0024: 8-61277849-61278098: 70.0%

12.2 Sequence

(SEQ ID NO: 23)

CATCATCTTCTGGCCCGGAGGCCTGTTAACATTTTAAGACCTTTTCTTCA
GAAAACTTATTTTTCTCTAAAGGTGGTTGGTCAGGAGCCACCCTCCAAAA
AAGCATTAGATAAAGTTGCATTCCTACAGAGCAAAGGTGTGGTGGGCTAT
AACAAGAAAAAGAATTAACTCAAGGGTCCCAGGTTACAAACATTAAAGCT
ACTACTTACACCAATTATATTAATCAATACACTGCCAGGGACACAGCAGG
TAAGGGATATGGAAACTTAGCAGCAAACAATGGCCCAACAAGTGAAAAAC
CATTCACCAATACAATTTCTAATCAATCTTGTAACTCCTCAAAAGAATCT
GTGTTTAGACAGTTTAGAACATCTCCTGCCTCTCACAGTTGGGAGGCTCT
GAAC

12.3 Sequence with 25 bp Surroundings

(SEQ ID NO: 24)

NNNNNNNAAAAGCTTAGGTGATTTGCATCATCTTCTGGCCCGGAGGCCTG
TTAACATTTTAAGACCTTTTCTTCAGAAAACTTATTTTTCTCTAAAGGTG
GTTGGTCAGGAGCCACCCTCCAAAAAAGCATTAGATAAAGTTGCATTCCT
ACAGAGCAAAGGTGTGGTGGGCTATAACAAGAAAAAGAATTAACTCAAGG
GTCCCAGGTTACAAACATTAAAGCTACTACTTACACCAATTATATTAATC
AATACACTGCCAGGGACACAGCAGGTAAGGGATATGGAAACTTAGCAGCA
AACAATGGCCCAACAAGTGAAAAACCATTCACCAATACAATTTCTAATCA
ATCTTGTAACTCCTCAAAAGAATCTGTGTTTAGACAGTTTAGAACATCTC
CTGCCTCTCACAGTTGGGAGGCTCTGAACAATCACATGTGGCCGGAAAAA
CCT

12.4 Annotation

12.4.1 Genes

### RefSeqID: NM_001192864

Gene name: ASB4
Product: ankyrin repeat and SOCS box protein 4
Translation start: 12597837

### Translation end: 12699701

### Exons: 5

12.4.2 Next Upstream Gene

### RefSeqID: NM_001013588

Gene name: PON2
Product: serum paraoxonase/arylesterase 2 precursor
Translation start: 12493205

### Translation end: 12520270 Distance to

region end: 106218 Exons: 9

12.4.3 Next Downstream Gene

### RefSeqID: NM_001101883

Gene name: PDK4
Product: pyruvate dehydrogenase kinase 4
Translation start: 12754201

### Translation end: 12767677 Distance to

region end: 127310 Exons: 11

12.4.4 Repeats

Repeat name: BTLTR1 Repeat
class: LTR Repeat family:
ERVK Repeat start: 12626470

### Repeat end: 12627280

13 bov-jd-0020: 4:51996600-51996918; Internal Reference Region

13.1 Similarities

15 bov-jd-0022: X-30978410-30978660: 100.0%

13.2 Sequence

(SEQ ID NO: 25)

ACCAAGTTTCGGTGAACAAGGCCCGCACTTTATTTTTCAAAGTAGTTTTT
ATACCTTAAGTTATGCATAGAGGATAATGGGGGAAGGGGTCGAGTCTTGC
AGCAAACCAGGCTTTCTTCCTGCAAACTTATCATATGCAAAAGCTTAGGT
GATTTGCATCATCTTCTGGCCCGGAGGCCTGTTAACATTTTAAGACCTTT
TCTTCAGAAAACTTATTTTTCTCTAAAGGTGGTTGGTCAGGAGCCACCCT
CCAAAAAAGCATTAGATAAAGTTGCATTCCTACAGAGCAAAGGTGTGGTG
GGCTATAACAAGAAAAAGA

13.3 Sequence with 25 bp Surroundings

(SEQ ID NO: 26)

GTAAGAGAAAGAACGACATGGTGAGACCAAGTTTCGGTGAACAAGGCCCG
CACTTTATTTTTCAAAGTAGTTTTTATACCTTAAGTTATGCATAGAGGAT
AATGGGGGAAGGGGTCGAGTCTTGCAGCAAACCAGGCTTTCTTCCTGCAA
ACTTATCATATGCAAAAGCTTAGGTGATTTGCATCATCTTCTGGCCCGGA
GGCCTGTTAACATTTTAAGACCTTTTCTTCAGAAAACTTATTTTTCTCTA
AAGGTGGTTGGTCAGGAGCCACCCTCCAAAAAAGCATTAGATAAAGTTGC
ATTCCTACAGAGCAAAGGTGTGGTGGGCTATAACAAGAAAAAGANNNNNN
NNNNNNNNNNNNNNNNNN

13.4 Annotation

13.4.1 Genes

### RefSeqID: NM_001012999

Gene name: MET
Product: hepatocyte growth factor receptor precursor
Translation start: 51912650

### Translation end: 52042198

### Exons: 22

13.4.2 Next Upstream Gene

### RefSeqID: NM_001012998

Gene name: CAPZA2
Product: F-actin-capping protein subunit alpha-2
Translation start: 51781843

### Translation end: 51836823 Distance to

region end: 159776 Exons: 10

Next downstream Gene

### RefSeqID: NM_174004

Gene name: CAVI Product:
caveolin-1 Translation start:
52173109

### Translation end: 52208687 Distance to

region end: 176192 Exons: 3

13.4.3 Repeats

Repeat name: BTLTR1 Repeat
class: LTR Repeat family:
ERVK Repeat start: 51996400

### Repeat end: 51996918

14 bov-jd-0021: 25:36122680-36122976; Internal Reference Region

14.1 Similarities

9 bov-jd-0009: 1-29318016-29318401: 96.0%

10 bov-jd-0010: 7-34812837-34813209: 73.7%

14.2 Sequence

(SEQ ID NO: 27)

ACACCTTTGCTCTGTAGGAATGCAACTTTATCTAATGCTTTTTTGGAGGG
TGGCTCCTGACCAACCACCTTTAGAGAAAAATAAGTTTTCTGAAGAAAAG
GTCTTAAAATGTTAACAGGCCTCCGGGCCAGAAGATGATGCAAATCACCT
AAGCTTTTGCATATGATAAGTTTGCAGGAAGAAAGCCTGGTTTGCTGCAA
GACTCGACCCCTTCCCCCATTATCCTCTATGCATAACTTAAGGTATAAAA
ACTACTTTGAAAAATAAAGTGCGGGCCTTGTTCACCGAAACTTGGTC

14.3 Sequence with 25 bp Surroundings

(SEQ ID NO: 28)

NNNNNNNNNNNNNNNNNNNNNNNNNACACCTTTGCTCTGTAGGAATGCAA
CTTTATCTAATGCTTTTTTGGAGGGTGGCTCCTGACCAACCACCTTTAGA
GAAAAATAAGTTTTCTGAAGAAAAGGTCTTAAAATGTTAACAGGCCTCCG
GGCCAGAAGATGATGCAAATCACCTAAGCTTTTGCATATGATAAGTTTGC
AGGAAGAAAGCCTGGTTTGCTGCAAGACTCGACCCCTTCCCCCATTATCC
TCTATGCATAACTTAAGGTATAAAAACTACTTTGAAAAATAAAGTGCGGG
CCTTGTTCACCGAAACTTGGTCTCACCATGTCGTTCTTTCTCTTAC

14.4 Annotation

14.4.1 Genes

14.4.2 Next Upstream Gene

### RefSeqID: NM_001193255

Gene name: PLOD3
Product: procollagen-lysine,2-oxoglutarate 5-dioxygenase 3 precursor Translation
start: 36057471

### Translation end: 36065405 Distance to

region end: 57274 Exons: 19

14.4.3 Next Downstream Gene

### RefSeqID: NM_001077110

Gene name: AP1S1
Product: AP-1 complex subunit sigma-1A
Translation start: 36180837

### Translation end: 36185662

Distance to region end: 57862 Exons: 4

14.4.4 Repeats

Repeat name: BTSAT4 Repeat
class: Satellite Repeat family:
centr Repeat start: 36121770

### Repeat end: 36124127

15 bov-jd-0022: X:30978436-30978636; Internal Reference Region

15.1 Similarities

13 bov-jd-0020: 4-51996574-51996942: 100.0%

15.2 Sequence

(SEQ ID NO: 29)

TGCATAGAGGATAATGGGGGAAGGGGTCGAGTCTTGCAGCAAACCAGGCT
TTCTTCCTGCAAACTTATCATATGCAAAAGCTTAGGTGATTTGCATCATC
TTCTGGCCCGGAGGCCTGTTAACATTTTAAGACCTTTTCTTCAGAAAACT
TATTTTTCTCTAAAGGTGGTTGGTCAGGAGCCACCCTCCAAAAAAGCATT
A

15.3 Sequence with 25 bp Surroundings

(SEQ ID NO: 30)

AAAGTAGTTTTTATACCTTAAATTATGCATAGAGGATAATGGGGGAAGGG
GTCGAGTCTTGCAGCAAACCAGGCTTTCTTCCTGCAAACTTATCATATGC
AAAAGCTTAGGTGATTTGCATCATCTTCTGGCCCGGAGGCCTGTTAACAT
TTTAAGACCTTTTCTTCAGAAAACTTATTTTTCTCTAAAGGTGGTTGGTC
AGGAGCCACCCTCCAAAAAAGCATTAGCCAGAGTTGCATTCCTACAGAGC

15.4 Annotation

15.4.1 Genes

15.4.2 Next Upstream Gene

### RefSeqID: NM_001192150

Gene name: FMR1
Product: fragile X mental retardation protein 1 Translation
start: 30922773

### Translation end: 30962111 Distance to

region end: 16324 Exons: 17

15.4.3 Next Downstream Gene

### RefSeqID: NM_001102262

Gene name: FMR1NB
Product: fragile X mental retardation 1 neighbor protein
Translation start: 30989739

### Translation end: 31029260 Distance to

region end: 11104 Exons: 6

15.4.4 Repeats

Repeat name: BTLTR1 Repeat
class: LTR Repeat family:
ERVK Repeat start: 30978173

### Repeat end: 30978969

16 bov-jd-0023: 28:40139956-40140405; Internal Reference Region

16.1 Similarities

28 bov-jd-0035: 27-26341465-26341714: 71.5%

10 bov-jd-0010: 7-34812837-34813209: 75.2%

16.2 Sequence

(SEQ ID NO: 31)

GAAATCCTCTAGCCTGCCTGAATAGGTTTTTCCGGCCACATGGGATTGTT
CAGAGCCTCCCAACTGTGAGAGGCAGGAGATGTTCTAAACTGTCTAAACA
CAGATTCTTTTGAGTAGTTACAAGATTGATTAGAAATTGTATTGGTGAAT
GGTTTTTCACTTCTTGGGCCATTGTTTGCTGCTAAGTTTCCATATCCCTT
ACCTGCTGTGTCCCTGGCAGTGTATTGATTAATATAATTGGTGTAAGTAG
TAGCTTTAATGTTTGTAACCTGGGACCCTTGAGTTAATTCTTTTTCTTGT
TATAGCCCACCACACCTTTGCTCTGTAGGAATGCAACTTTATCTAATGCT
TTTTTGGAGGGTGGCTCCTGACCAACCACCTTTAGAGAAAAATAAGTTTT
CTGAAGAAAAGGTCTTAAAATGTTAACAGGCCTCCGGGCCAGAAGATGAT

16.3 Sequence with 25 bp Surroundings

(SEQ ID NO: 32)

AGTGTTTAACCTACAAACTCCTTTGGAAATCCTCTAGCCTGCCTGAATAG
GTTTTTCCGGCCACATGGGATTGTTCAGAGCCTCCCAACTGTGAGAGGCA
GGAGATGTTCTAAACTGTCTAAACACAGATTCTTTTGAGTAGTTACAAGA
TTGATTAGAAATTGTATTGGTGAATGGTTTTTCACTTCTTGGGCCATTGT
TTGCTGCTAAGTTTCCATATCCCTTACCTGCTGTGTCCCTGGCAGTGTAT
TGATTAATATAATTGGTGTAAGTAGTAGCTTTAATGTTTGTAACCTGGGA
CCCTTGAGTTAATTCTTTTTCTTGTTATAGCCCACCACACCTTTGCTCTG
TAGGAATGCAACTTTATCTAATGCTTTTTTGGAGGGTGGCTCCTGACCAA
CCACCTTTAGAGAAAAATAAGTTTTCTGAAGAAAAGGTCTTAAAATGTTA
ACAGGCCTCCGGGCCAGAAGATGATGCAAATNNNNNNNNNNNNNNNNN

16.4 Annotation

16.4.1 Genes

16.4.2 Next Upstream Gene

### RefSeqID: NM_175775

Gene name: RGR
Product: RPE-retinal G protein-coupled receptor
Translation start: 39531570

### Translation end: 39542180 Distance to

region end: 597775 Exons: 7

16.4.3 Next Downstream Gene

### RefSeqID: NM_031210

Gene name: MIR346 Product:
Translation start: 41329054

### Translation end: 41329149 Distance to

region end: 1188650 Exons: 1

16.4.4 Repeats

Repeat name: BTLTR1 Repeat
class: LTR Repeat family:
ERVK Repeat start: 40139561

### Repeat end: 40140411

17 bov-jd-0024: 8:61277875-61278074; Internal Reference Region

17.1 Similarities

24 bov-jd-0031: 22-42434551-42434800: 76.0%

12 bov-jd-0012: 4-12626463-12626916: 70.0%

17.2 Sequence

(SEQ ID NO: 33)

ACCTTAGAACAAACCGAGGCACTATGAACATTTTGTGCTTCATGTTGATG
ACTCTTAGACATGTCTACAGTAGAGGAGCAAAAACAAAACTACTAGATAT
TTCATATTGACTAGTTCCCAGTTCACGGGACTCTGACATTCCCTGAGGTC
AAAGTTTTCTTGTATTGGAAGCAGTTGGGTTTGCAAGGGCTGCCTTGTCT

17.3 Sequence with 25 bp Surroundings

(SEQ ID NO: 34)

CAGATAGAACGATATAGCAATTTTTACCTTAGAACAAACCGAGGCACTAT
GAACATTTTGTGCTTCATGTTGATGACTCTTAGACATGTCTACAGTAGAG
GAGCAAAAACAAAACTACTAGATATTTCATATTGACTAGTTCCCAGTTCA
CGGGACTCTGACATTCCCTGAGGTCAAAGTTTTCTTGTATTGGAAGCAGT
TGGGTTTGCAAGGGCTGCCTTGTCTTGAGACCATTGAAATAAGAACTCA

17.4 Annotation

17.4.1 Genes

17.4.2 Next Upstream Gene

### RefSeqID: NM_001111260

Gene name: MELK
Product: maternal embryonic leucine zipper kinase
Translation start: 61157468

### Translation end: 61230549 Distance to

region end: 47325 Exons: 18

17.4.3 Next Downstream Gene

### RefSeqID: NM_001046100

Gene name: ZCCHC7
Product: zinc finger CCHC domain-containing protein 7
Translation start: 61611660

### Translation end: 61875342 Distance to

region end: 333587 Exons: 9

17.4.4 Repeats

18 bov-jd-0025: 6:19804114-19804313; Internal Reference Region

18.1 Similarities

22 bov-jd-0029: 27-23691655-23691904: 96.0%

28 bov-jd-0035: 27-26341465-26341714: 80.0%

18.2 Sequence

(SEQ ID NO: 35)

AGAAAACTTTGACCTCAGGGAATGTCAGAGTCCCGTGAACTGGGAACTAG
TCAATATGAAATATCTAGTAGTTTTGTTTTTGCTCCTCTACTGTAGACAT
GTCTAAGAGTCATCAACATGAAGCACAAAATGTTCATAGTGCCTCGGTTT
GTTCTAAGGTAAAAATTGCTATTGCTATATCGTTCTATCTGTCACAAGCT

18.3 Sequence with 25 bp Surroundings

(SEQ ID NO: 36)

TGCAAACCCAACTGCTTCCAATACAAGAAAACTTTGACCTCAGGGAATGT
CAGAGTCCCGTGAACTGGGAACTAGTCAATATGAAATATCTAGTAGTTTT
GTTTTTGCTCCTCTACTGTAGACATGTCTAAGAGTCATCAACATGAAGCA
CAAAATGTTCATAGTGCCTCGGTTTGTTCTAAGGTAAAAATTGCTATTGC
TATATCGTTCTATCTGTCACAAGCTTGTCAACAAGAAAGTACCTCGAAA

18.4 Annotation

18.4.1 Genes

18.4.2 Next Upstream Gene

### RefSeqID: NM_001082615

Gene name: DKK2
Product: dickkopf-related protein 2 precursor
Translation start: 19381412

### Translation end: 19511150 Distance to

region end: 292963 Exons: 4

18.4.3 Next Downstream Gene

### RefSeqID: NM_001035018

Gene name: AIMP1
Product: aminoacyl tRNA synthase complex-interacting multifunctional protein 1 Translation
start: 20111143

### Translation end: 20138597 Distance to

region end: 306831 Exons: 7

18.4.4 Repeats

19 bov-jd-0026: 27:26332496-26332695; Internal Reference Region

19.1 Similarities

20 bov-jd-0027: 28-30011085-30011334: 95.5%

19.2 Sequence

(SEQ ID NO: 37)

TGGACCCCAGAAAAGAGTGTTGTGCCTAGCACAAGGTTTCTTGAGAGGTT
GAACTGCCTTTGCTAATGGATTTTAAGTTTCTTTACCTCTTCCGTGATCT
CTTCTAGGTTGACCTAGAAGAGAAATCTTTTGTTAGCTTTGGCTAAGTGG
AGAAGTATTGCTTCACGGTGACTTGTGTGATCCTACCTGACTGTTCTAAA

19.3 Sequence with 25 bp Surroundings

(SEQ ID NO: 38)

TGGGATCAGGAGGTTGTAGGGAACGTGGACCCCAGAAAAGAGTGTTGTGC
CTAGCACAAGGTTTCTTGAGAGGTTGAACTGCCTTTGCTAATGGATTTTA
AGTTTCTTTACCTCTTCCGTGATCTCTTCTAGGTTGACCTAGAAGAGAAA
TCTTTTGTTAGCTTTGGCTAAGTGGAGAAGTATTGCTTCACGGTGACTTG
TGTGATCCTACCTGACTGTTCTAAACCCTTTTGATATCTATGCACTGCT

19.4 Annotation

19.4.1 Genes

### RefSeqID: NM_001143857

Gene name: WRN
Product: Werner syndrome ATP-dependent helicase
Translation start: 26263002

### Translation end: 26449795

### Exons: 34

19.4.2 Next Upstream Gene

### RefSeqID: NM_001075857

Gene name: PPP2CB
Product: serine/threonine-protein phosphatase 2A catalytic subunit beta isoform Translation
start: 26041252

### Translation end: 26064654 Distance to

region end: 267841 Exons: 7

19.4.3 Next Downstream Gene

### RefSeqID: NM_174128

Gene name: NRG1
Product: pro-neuregulin-1, membrane-bound isoform
Translation start: 27623937
Translation end: 27833477 Distance to region end: 1291243

### Exons: 7

19.4.4 Repeats

20 bov-jd-0027: 28:30011111-30011310; Internal Reference Region

20.1 Similarities

19 bov-jd-0026: 27-26332470-26332719: 95.5%

20.2 Sequence

(SEQ ID NO: 39)

CAGAAAAGAATGTTGTGCCTAGCACAAGGTTTCTTGAGAGGTTGAACTGC
CTTTGCTAATGGATTTTAAGTTTCTTTACCTCTTCCGTGATCTCTTCTAG
GTTGACCTAGAACAGAAATCTTTTGTTAGCTTTGGCTAAGTGGAGAAGTA
TTGCTTCACGGTGACTTGTGTGATCCTACCTGACTGTTCTAAACCCTTTT

20.3 Sequence with 25 bp Surroundings

(SEQ ID NO: 40)

GGAGGTTGTAGGGATAGTGGACCCCCAGAAAAGAATGTTGTGCCTAGCAC
AAGGTTTCTTGAGAGGTTGAACTGCCTTTGCTAATGGATTTTAAGTTTCT
TTACCTCTTCCGTGATCTCTTCTAGGTTGACCTAGAACAGAAATCTTTTG
TTAGCTTTGGCTAAGTGGAGAAGTATTGCTTCACGGTGACTTGTGTGATC
CTACCTGACTGTTCTAAACCCTTTTGATATCTATGCACTGTTGCTGCTG

20.4 Annotation

20.4.1 Genes

20.4.2 Next Upstream Gene

### RefSeqID: NM_174147

Gene name: PLAU
Product: urokinase-type plasminogen activator precursor
Translation start: 29964982

### Translation end: 29971029 Distance to

region end: 40081 Exons: 11

20.4.3 Next Downstream Gene

### RefSeqID: NM_001191370

Gene name: VCL Product:
vinculin
Translation start: 30053805

### Translation end: 30166316 Distance to

region end: 42496 Exons: 22

20.4.4 Repeats

Repeat name: (TGC)n Repeat class:
Simple repeat Repeat family:
Simple repeat Repeat start:
30011326

### Repeat end: 30011356

21 bov-jd-0028: 4:103646990-103647189; Internal Reference Region

21.1 Similarities

26 bov-jd-0033: X-65494632-65494881: 85.5%

21.2 Sequence

(SEQ ID NO: 41)

GTATCCTGGCACATACATGTACTGCCTCACCCCTAACCTCAGTCATGTGT
TCCAAGAACTTCTGTCCGTTTATTTATTTGTTTTTGGTAACATTGAATTC
GTTTTGACTATGCTGGGTCTTTGCTGCTCGCTGGAAGTTGCTAGGACCTC
TGGACTGCAGTCTCCCATCTCTTGCTGACCTCTTGTTGACCTCTGCAGAA

21.3 Sequence with 25 bp Surroundings

(SEQ ID NO: 42)

TCTACGTTAAGAGTATACTTTAAGAGTATCCTGGCACATACATGTACTGC
CTCACCCCTAACCTCAGTCATGTGTTCCAAGAACTTCTGTCCGTTTATTT
ATTTGTTTTTGGTAACATTGAATTCGTTTTGACTATGCTGGGTCTTTGCT
GCTCGCTGGAAGTTGCTAGGACCTCTGGACTGCAGTCTCCCATCTCTTGC
TGACCTCTTGTTGACCTCTGCAGAATCCTGGTTGGTGGGAGCTTGTTAG

21.3.1 Annotation

21.3.2 Genes

### RefSeqID: NM_001193208

Gene name: UBN2 Product:
ubinuclein-2
Translation start: 103594500

### Translation end: 103689157

### Exons: 18

21.3.3 Next Upstream Gene

### RefSeqID: NM_001192577

Gene name: ZC3HAV1L
Product: zinc finger CCCH-type antiviral protein 1-like
Translation start: 103454360

### Translation end: 103465193 Distance to

region end: 181796 Exons: 5

21.3.4 Next Downstream Gene

### RefSeqID: NM_001076997

Gene name: FMC1
Product: protein FMC1 homolog Translation start:
103715431

### Translation end: 103719743 Distance to

region end: 68243 Exons: 2

21.3.5 Repeats

Repeat name: ERV1-1C-LTR_BT Repeat class:

### LTR

Repeat family: ERV1 Repeat start:
103646797

### Repeat end: 103646974

22 bov-jd-0029: 27:23691681-23691880; Internal Reference Region

22.1 Similarities

28 bov-jd-0035: 27-26341465-26341714: 80.5%

18 bov-jd-0025: 6-19804088-19804337: 96.0%

22.2 Sequence

(SEQ ID NO: 43)

ACAAGAAAACTTTGACCTCAGGGAATGTCAGAGTCCCGTGAACTGGGAAC
TAGTCAATATGAAATATCTAGTAGTTTTGTTTTTGCTCCTCTACTGTAGA
CATGTCTAAGAGTCATCAACACGAAGCACAAAATGTTCATAGTGCCTCGG
TTTGTTCTGAGGTAAAAATTGCTATATCGTTCTATCTGTCACAAGCTTGT

22.3 Sequence with 25 bp Surroundings

(SEQ ID NO: 44)

CCTTGCAAACCCAACTGCTTCCAATACAAGAAAACTTTGACCTCAGGGAA
TGTCAGAGTCCCGTGAACTGGGAACTAGTCAATATGAAATATCTAGTAGT
TTTGTTTTTGCTCCTCTACTGTAGACATGTCTAAGAGTCATCAACACGAA
GCACAAAATGTTCATAGTGCCTCGGTTTGTTCTGAGGTAAAAATTGCTAT
ATCGTTCTATCTGTCACAAGCTTGTCAACAAGAAAGTACCTCGAAAGAA

22.4 Annotation

22.4.1 Genes

22.4.2 Next Upstream Gene

### RefSeqID: NM_001076832

Gene name: TRMT9B
Product: probable tRNA methyltransferase 9-like protein
Translation start: 23063309

### Translation end: 23137025 Distance to

region end: 554655 Exons: 6

22.4.3 Next Downstream Gene

### RefSeqID: NM_001038192

Gene name: ERI1
Product: 3′-5′ exoribonuclease 1
Translation start: 24193121

### Translation end: 24209947 Distance to

region end: 501242 Exons: 7

22.4.4 Repeats

23 bov-jd-0030: 27:23723113-23723312; Internal Reference Region

23.1 Sequence

(SEQ ID NO: 45)

AGCCTGCCTAAATCATTGACTTCTAGCCACCTTTGGGATGCCAATGACCA
CCCAGCCTTACCAGGCCACAGAGGAGAGAAAACATTTACCACATGAGGTC
CGGGTAAGGAACAAGGAACTAACGAGCTCCCACCAACCAGGATTCTGCAG
AGGTCAACAAGAGGTCAGCAAGAGATGGGAGACTGCAGTCCAGAGGTCCT

23.2 Sequence with 25 bp Surroundings

(SEQ ID NO: 46)

TTGCCTTCTCCCATATCTATTCACAAGCCTGCCTAAATCATTGACTTCTA
GCCACCTTTGGGATGCCAATGACCACCCAGCCTTACCAGGCCACAGAGGA
GAGAAAACATTTACCACATGAGGTCCGGGTAAGGAACAAGGAACTAACGA
GCTCCCACCAACCAGGATTCTGCAGAGGTCAACAAGAGGTCAGCAAGAGA
TGGGAGACTGCAGTCCAGAGGTCCTAGCAAATTCCAGCGAGCAGCTAAG

23.3 Annotation

23.3.1 Genes

23.3.2 Next Upstream Gene

### RefSeqID: NM_001076832

Gene name: TRMT9B
Product: probable tRNA methyltransferase 9-like protein
Translation start: 23063309

### Translation end: 23137025 Distance to

region end: 586087 Exons: 6

23.3.3 Next Downstream Gene

### RefSeqID: NM_001038192

Gene name: ERI1
Product: 3′-5′ exoribonuclease 1
Translation start: 24193121

### Translation end: 24209947 Distance to

region end: 469810 Exons: 7

23.3.4 Repeats

Repeat name: BOV-A2 Repeat
class: SINE Repeat family: Core-RTE
Repeat start: 23722838

### Repeat end: 23723098

24 bov-jd-0031: 22:42434577-42434776; Internal Reference Region

24.1 Similarities

17 bov-jd-0024: 8-61277849-61278098: 76.0%

24.2 Sequence

(SEQ ID NO: 47)

TGACTCTTAGACATGTCTACAGTAGAGGAGCAAAAACAAAACTACTAGAT
ATTTCATATTGACTAGTTCCCAGTTCACGGGACTCTGACATTCCCTGAGG
TCAAAGTTTTCTTGTATTGGAAGCAGTTGGGTTTGCAAGGGCTGCCTTGT
CTTGAGACCATTGAACTAAGAACTCAGAACTTGAGCACTATTATCAAAAA

24.3 Sequence with 25 bp Surroundings

(SEQ ID NO: 48)

ATGAACATTTTGTGCTTCATGTTGATGACTCTTAGACATGTCTACAGTAG
AGGAGCAAAAACAAAACTACTAGATATTTCATATTGACTAGTTCCCAGTT
CACGGGACTCTGACATTCCCTGAGGTCAAAGTTTTCTTGTATTGGAAGCA
GTTGGGTTTGCAAGGGCTGCCTTGTCTTGAGACCATTGAACTAAGAACTC
AGAACTTGAGCACTATTATCAAAAATCACAAGGCTCACACTGACACAGA

24.4 Annotation

24.4.1 Genes

24.4.2 Next Upstream Gene

### RefSeqID: NM_001040646

Gene name: FHIT
Product: bis(5′-adenosyl)-triphosphatase Translation
start: 40603978

### Translation end: 42134601 Distance to

region end: 299975 Exons: 9

24.4.3 Next Downstream Gene

### RefSeqID: NM_001083488

Gene name: FAM107A Product:

protein FAM107A Translation
start: 43354327

### Translation end: 43359047 Distance to

region end: 919552 Exons: 4

24.4.4 Repeats

25 bov-jd-0032: 20:2742-294; Internal Reference Region

25.1 Sequence

(SEQ ID NO: 49)

TTGGGTGTCACAGTGGAGGTGTAGAAGAGGTCTGAGAGTGCAAGGCAGTT
AAGGAAGAAGTACATGGGCTGGGTGATTAGCTGACTGCATGTAATAGAAA
TCACAATGATCAAATTCCCCAACCAAATAGCCAGGTAACAGAATAAGAAA
AATAAAAAGCAGAGGATCTGGACTTTCTTGTCCATAGAAAGTTCTAAGAG

25.2 Sequence with 25 bp Surroundings

(SEQ ID NO: 50)

CTCCATCAGTAAGTCAGTCATGAGTTTGGGTGTCACAGTGGAGGTGTAGA
AGAGGTCTGAGAGTGCAAGGCAGTTAAGGAAGAAGTACATGGGCTGGGTG
ATTAGCTGACTGCATGTAATAGAAATCACAATGATCAAATTCCCCAACCA
AATAGCCAGGTAACAGAATAAGAAAAATAAAAAGCAGAGGATCTGGACTT
TCTTGTCCATAGAAAGTTCTAAGAGGATAAATTCAGTAACATTATTTCT

25.3 Annotation

25.3.1 Genes

25.3.2 Next Downstream Gene

### RefSeqID: NM_001040540

Gene name: SPZ1
Product: spermatogenic leucine zipper protein 1 Translation
start: 153401

### Translation end: 155115 Distance to

region end: 150461 Exons: 1

25.3.3 Repeats

26 bov-jd-0033: X:65494658-65494857; Internal Reference Region

26.1 Similarities

21 bov-jd-0028: 4-103646964-103647213: 85.5%

26.2 Sequence

(SEQ ID NO: 51)

CACCCCTAACCTCAGTCGTGTGTTCCAAGAACTTCTGTCCGTTTATTTAT
TTGTTTTGGGTAACATTGAATTCGTTTTGACTATGCTGGGTCTTTGCTGC
TCGCTGGAAGTTGCTAGGACCTCTGGACTGCAGTCTCCCATCTCTTGCTG
ACCTCTTGTTGACCTCTGCAGAATCCTGGTTGCTGGGAGCTTGTTAGTTC

26.3 Sequence with 25 bp Surroundings

(SEQ ID NO: 52)

ATCCTGGCACATACATGTACTGCCTCACCCCTAACCTCAGTCGTGTGTTC
CAAGAACTTCTGTCCGTTTATTTATTTGTTTTGGGTAACATTGAATTCGT
TTTGACTATGCTGGGTCTTTGCTGCTCGCTGGAAGTTGCTAGGACCTCTG
GACTGCAGTCTCCCATCTCTTGCTGACCTCTTGTTGACCTCTGCAGAATC
CTGGTTGCTGGGAGCTTGTTAGTTCCTTGTTCCTTACCCGGACCACCTG

26.4 Annotation

26.4.1 Genes

26.4.2 Next Upstream Gene

### RefSeqID: NM_001166574

Gene name: TRPCS
Product: short transient receptor potential channel 5
Translation start: 65246770

### Translation end: 65449919 Distance to

region end: 44738 Exons: 10

26.4.3 Next Downstream Gene

### RefSeqID: NM_031235

Gene name: MIR764 Product:
Translation start: 67800434

### Translation end: 67800529 Distance to

region end: 2305578 Exons: 1

26.4.4 Repeats

27 bov-jd-0034: 27:26331117-26331316; Internal Reference Region

27.1 Sequence

(SEQ ID NO: 53)

CAAGAGGTCAGCAAGAGATGGGAGACTGCAGTCCAGAGGTCCTAGCAACT
TCCAGCGAGCAGCAAAGACCCAGCATAGTCAAAACGAATTCAATGTTACC
AAAAACAAATAAATAAACGGACAGAAGTTCTTGGAACACATGACTGAGGT
TAGGGGTGAGGCAGTACATGTATGTGCCAGGATACTCTTAAAGTATACTC

27.2 Sequence with 25 bp Surroundings

(SEQ ID NO: 54)

CCAACCAGGATTCTGCAGAGGTCAACAAGAGGTCAGCAAGAGATGGGAGA
CTGCAGTCCAGAGGTCCTAGCAACTTCCAGCGAGCAGCAAAGACCCAGCA
TAGTCAAAACGAATTCAATGTTACCAAAAACAAATAAATAAACGGACAGA
AGTTCTTGGAACACATGACTGAGGTTAGGGGTGAGGCAGTACATGTATGT
GCCAGGATACTCTTAAAGTATACTCTTAACGTAGATGAGGAGAACAAGG

27.3 Annotation

27.3.1 Genes

### RefSeqID: NM_001143857

Gene name: WRN
Product: Werner syndrome ATP-dependent helicase
Translation start: 26263002

### Translation end: 26449795

### Exons: 34

27.3.2 Next Upstream Gene

### RefSeqID: NM_001075857

Gene name: PPP2CB
Product: serine/threonine-protein phosphatase 2A catalytic subunit beta isoform Translation
start: 26041252

### Translation end: 26064654 Distance to

region end: 266462 Exons: 7

27.3.3 Next Downstream Gene

### RefSeqID: NM_174128

Gene name: NRG1
Product: pro-neuregulin-1, membrane-bound isoform
Translation start: 27623937

### Translation end: 27833477 Distance to

region end: 1292622 Exons: 7

27.3.4 Repeats

Repeat name: ERV1-1C-LTR BT Repeat class:

### LTR

Repeat family: ERV1 Repeat start:
26331316

### Repeat end: 26331494

28 bov-jd-0035: 27:26341491-26341690; Internal Reference Region

28.1 Similarities

22 bov-jd-0029: 27-23691655-23691904: 80.5%

16 bov-jd-0023: 28-40139930-40140429: 71.5%

18 bov-jd-0025: 6-19804088-19804337: 80.0%

28.2 Sequence

(SEQ ID NO: 55)

GACAAGGCAGCCCTTGCAAACCCAACTGCTTCCAATACAAGAAAACTTTG
ACCTCAGGGAATGTCAGAGTCCCGTGAACTGGGAACTAGTCAATATGAAA
TATCTGGTAGTTTTGTTTTTGCTCCTCTACTGTAGACATGTCTAAGAGTC
ATCAACATGAAGCACAAAATGTTCATAGTGCCTCGGTTTGTTCTAAGGTA

28.3 Sequence with 25 bp Surroundings

(SEQ ID NO: 56)

TGAGTTCTTATTTCAATGGTCTCAAGACAAGGCAGCCCTTGCAAACCCAA
CTGCTTCCAATACAAGAAAACTTTGACCTCAGGGAATGTCAGAGTCCCGT
GAACTGGGAACTAGTCAATATGAAATATCTGGTAGTTTTGTTTTTGCTCC
TCTACTGTAGACATGTCTAAGAGTCATCAACATGAAGCACAAAATGTTCA
TAGTGCCTCGGTTTGTTCTAAGGTAAAAATTGCTATATCGTTCTATCTG

28.4 Annotation

28.4.1 Genes

### RefSeqID: NM_001143857

Gene name: WRN
Product: Werner syndrome ATP-dependent helicase
Translation start: 26263002

### Translation end: 26449795

### Exons: 34

28.4.2 Next Upstream Gene

### RefSeqID: NM_001075857

Gene name: PPP2CB
Product: serine/threonine-protein phosphatase 2A catalytic subunit beta isoform Translation
start: 26041252

### Translation end: 26064654 Distance to

region end: 276836 Exons: 7

28.4.3 Next Downstream Gene

### RefSeqID: NM_174128

Gene name: NRG1
Product: pro-neuregulin-1, membrane-bound isoform Translation start: 27623937

### Translation end: 27833477 Distance to

region end: 1282248 Exons: 7

28.4.4 Repeats

### Sequence J139 (Over-Represented)

(SEQ ID NO: 134)

GAGTATTCCTCTGTCTCTTCATCTTGTTTAAATTGGAATCACAGATAAAT
AGCCTGGAGACAAGGATTGAGAAGATGCAAGAAAGGTTTAACAAGGACCT
AGAAGAAATAAAAAAGAGTCAATATATAATGAATAATGCAATAAATGACA
TTAAAAACACTCTGGAGGCAACAAATAGTAGAATAACAGAGGCAGAAGAT
AGGATTAGTGAATTAGAAGATAGAATGGTAGAAATAAATG

### Location

### Assembly Bos_taurus_UMD_3.1.1

chr22:50,204,119-50,204,340

### Annotation

### RepeatMasker Information

### Name: L1 BT

### Family: L1

### Class: LINE

### Sequence J141 (Over-Represented)

(SEQ ID NO: 135)

ATGGCACCCAGCCTTACCAGGCCACAGAGGAGAGAAAACACTTAACCACA
GGTGGTCCGGGTAAGGAACAAGGAACTAACAAGCTCCCAGCAACCAGGAT
TCTGCAGAGGTCAACAAGAGGTCAGCAAGAGATGGGAGACTGCAGTCCAG
AGGTCCTAGCAACTTCCAGCGAGCAGCAAAGACCCACTAACCC

### Location

### Assembly Bos_taurus_UMD_3.1.1

chrX:65,494,744-65,494,925

### Annotation

### RepeatMasker Information

### Sequence J143 (Over-Represented)

(SEQ ID NO: 136)

GTTTGGGTTGTGTTGTTAGTTCCTTGTTCCTTACCCGGACCACCTGTGGT
TAAGTGTTTTCTCTCCTCTGTGGCCTGGTAAGGCTGGGTGGTCATTGGCA
TCCCAAAGGTGGCTAGAGGTCAATGATTTAGGCAGGCTTGTGAATAGATA
TGGGAGAAGGCAATGGCACCCCACTCCAGTACTCTTGCCTGGAAAATCCC
ATGGACCCAAGAGCCTGGTAGGCTGCAGTCCATGGGGTCACTAGGAGT

### Location

### Assembly Bos_taurus_UMD_3.1.1

chr27:23,716,727-23,716,953

### Annotation

### RepeatMasker Information

### Name: BOV-A2

### Family: Core-RTE

### Class: SINE

### Sequence J145 (Over-Represented)

(SEQ ID NO: 137)

ACATGGAGAAGAGAAGAGGGAGGAGGGAGTTAGAAACTTTTCATCTTTAA
CGTAGATGTTTTATCAATGGTGCTGTATAGAAGGAGAAGTTTTGAAACTA
CTGTAAAAATAAGACCGATAACCGGAAGCAGGAGACTTAAGTCCAAACCC
TGACTCCAGGGAACTCCTGACTCCAAGGAACATTAATTGACGGGAGCTCA
TCAAATGCATCCATACCGACACTGAAACAAAGCACCACACAAGGGCCAAT
AAATTCCAG

### Location

### Assembly Bos_taurus_UMD_3.1.1

chr26:5,229,059-5,229,286

### Annotation

### RepeatMasker Information

### Name: L1 BT

### Family: L1

### Class: LINE

### Sequence J146 (Over-Represented)

(SEQ ID NO: 138)

GTCTCCTGTAGACAACATATGTAGGGGTCTTGTTTTTGTATCCATTCAGC
CAGTCTTTGTCTTTTGGTTGGGGCATTCAACCCATTTACGTTTAAGGTAA
TTACTGATAAGTATGATCCCGTTGCCATTTACTTTATTGTTTGGGGTTCG
AATTTATACACCATTTTTGTGTTTCCTGTCTAGAGAATATCCTTTAGTAT
TTGTTGGAGAGCTGGTTTGGTGGTGCAGAATTCTCTCAGCTTTTGCTTGT
CTGAGAAGCTTTTGATTTCTCCTTCATACTTGAATGAGATCC

### Location

### Assembly Bos_taurus_UMD_3.1.1

chr6:19,478,840-19,479,435

### Annotation

### Genes

### RefSeq: NM_001082615.1

Description: Bos taurus dickkopf WNT signaling pathway inhibitor 2 (DKK2), mRNA.

### Gene Symbol: DKK2

### Repeats

### Name: L1 BT

### Family: L1

### Class: LINE

### Sequence J147 (Over-Represented)

(SEQ ID NO: 139)

GTATAACCCCGTGAACATCTCTGAGTGGTCCAGTTGTGGAGTGCACATAA
GGAAGTGACTACTGGCTAGCCCACTCTCTCCACTATTGATTCACCTCATC
TCATTTGGGTCACCTCTAACTCCCTCCTCCCTCTTCTCTTCTCCATGTAA
CCCTGTGAACCTCTCTGAGTGACCCTCACTGTAGAGAAACTTATCATCTT
TAATGTAGATGTTTTATCAATGGTGCTGTATAGAAG

### Location

### Assembly Bos_taurus_UMD_3.1.1

chr9:90,778,334-90,778,569

### Annotation

### Repeats

### Name: L1 BT

### Family: L1

### Class: LINE

### Sequence J148 (Over-Represented)

(SEQ ID NO: 140)

ATTCTATCTTCTAATTCACTAATCCTATCTTCTGCCTCTGTTATTCTACT
ATTTGTTGCCTCCAGAGTGTTTTTAATTTCATTTATTGCATTATTCATTA
TATATTGACTCTCTTTTATTTCTTCTAGGTCCTTGTTAAACCTTTCTTGC
ATCTTCTCGATCTTAGTCTCCAAGCTATTTATCTGTGATTCCATTTTGAT
TTCAAGATTTTGGATCAATTTCACTATCATTATAGGGAATCTACCTGATA
AAGAATTCCAAATAA

### Location

### Assembly Bos_taurus_UMD_3.1.1

chr5:91,534,317-91,534,549

### Annotation

### Repeats

### Name: L1 BT

### Family: L1

### Class: LINE

### Sequence J149 (Over-Represented)

(SEQ ID NO: 141)

ATTTTATCTGATATGAGTATTGCTACTCCGGCTCTCCAACAAATACTAAA
GGATATTCTCTAGACAGGAAACACAAAAAGGGTGTATAAATTCGAACCCA
AAACAATAAAGTAAATGGCAACGGGATCATACTTATCAATAATTACCTTA
AACGTAAATGGGTTGAATGCCCCAACCAAAAGACAAAGACTGGCTGAATG
GATACAAAAACAAGACCCCTACATATGTTGTCTACAAGAGACCCACTTCA
GACCTAGGGAC

### Location

### Assembly Bos_taurus_UMD_3.1.1

chr1:35,411,734-35,411,952

### Annotation

### Repeats

### Name: L1M2

### Family: L1

### Class: LINE

### Sequence J150 (Over-Represented)

(SEQ ID NO: 142)

CTTCTATACAGCACCATTGATAAAACATCTACGTTAAAGATGAACAGTTT
CTCTACTGTGAGGGTCACCCAGAGAGGTTCACAGCGTTACATGGAGAAGA
GAAGAGGGAGGAGGGAGTTAGAGGTGACCCGAATGAGATGAGGTGGAATC
AATAGTGGAGTGAGTGGACTAGCCAATAATCACTTCCTTATGTGCACTCC
ACAACTGGACCGCTCATAGATGTTCACAGAGTTATACAG

### Location

### Assembly Bos_taurus_UMD_3.1.1

chr6:5,515,497-5,515,735

### Annotation

### Repeats

### Name: L1 BT

### Family: L1

### Class: LINE

### Sequence J151 (Over-Represented)

(SEQ ID NO: 143)

GTTTCACATTGTCCCAGAGGTCTCTGAGGTTGTCCTAATTTCTTATAATT
CTTTTTCCTTGTTTCCTCTCTGCTTCATTTATTTCTACCATTCTATCTTC
TACCTCACTTATCCTATCTTCTGCCTCTGTTATTCTACTATTTGTTGCCT
CCAGAGTGTTTTTAATTTCATTTATTGCATTATTCATTATATATTGACTC
TTTTTTATTTCTTCTAGGTCCTTGTTAAACCTTTCTTGCATCTTCTC

### Location

### Assembly Bos_taurus_UMD_3.1.1

chr6:5,833,268-5,833,514

### Annotation

### Repeats

### Name: L1-3 BT

### Family: L1

### Class: LINE

### Sequence J153 (Over-Represented)

(SEQ ID NO: 144)

GTTTGGGTTTGTTGTTTGATTAATATGTGTCTTGGGGTGTTTCCCCTTGG
GTTTATCCTGTTTGGGACTCTCTGGTTTTCTTGGACTTGGGTGATTATTT
CCTTCCCCATTTTAGGGAAGTTTTCAACTATTATCTCCTCAAGTATTTTC
TCATGGTCTTTCTTTTTGTCTTCTTCTTCTGGGACCCCTATGATTCGAAT
GTTGTAGCGTTTAATATTGTCCTGGAGGTCTCTGAGATTGTCCTCATTTC
TTTTAATTCGTTTTTCTTTTATTTTCTC

### Location

### Assembly Bos_taurus_UMD_3.1.1

chr23:1,113,953-1,114,617

### Annotation

### Repeats

### Name: L1 BT

### Family: L1

### Class: LINE

### Sequence JC83 (Under-Represented)

(SEQ ID NO: 145)

TTCCTGTGGTCATGTATGGATGTGAGAGTTGGACTGTGAAGAAGGCTGAG
CGCCGAAGAATTGATGCTTTTGAACTGTGGTGTTGGAGAAGACTCTAGAG
AGTCCCTTGGACTGCAAGGAGATCCAACCAGTCCATCCTAAAGGAGATCA
GTCCTGCGTGTTGATTGGAAGGAATGATGCTAAAGCTGAAACTCCAGTAC
TTTGACCACACA

### Location

### Assembly Bos_taurus_UMD_3.1.1

chr8:34,607,129-34,607,338

### Annotation

### Repeats

### Name: BovB

### Family: RTE-BovB

### Class: LINE

### Sequence JC86 (Under-Represented)

(SEQ ID NO: 146)

TTTCAAATGAGTCAGCTCTTCGCATCAGGTGGACAAAATATTGGAGTTTC
AGCTTCAACATCAGGACTGATTTCCTTTAGAATGGACTGGTTGGATCTCC
TTGCAGTCCAAGGGACTCTCAAGAGTCTTCTCCAACACCACAGTTCAAAA
GCATCAATTCTTCAGCACTCAGCTTTCTTCACAGTCCAACTCTCACATCC
AT

### Location

### Assembly Bos_taurus_UMD_3.1.1

chr11:60,951,570-61,196,616

### Annotation

### Repeats

### Name: BovB

### Family: RTE-BovB

### Class: LINE

### Sequence JC88 (Under-Represented)

(SEQ ID NO: 147)

GATGTGAGAGTTGGACTGTGAAGAAGGCTGAGCGCCGAAGAATTGATGCT
TTTGAACTGTGGTGTTGGAGAAGACTCTTGAGAGTCCTTTGGACTGCAAG
GAGATCCAACCAGTCCATTCTAAAGGAGATCAGCCCTGGGATTCCTTTGG
AGGGAATGATGCTAAAGCTGAAACTCCAATACTTTGGCCACCTGATGCAA
AGAAC

### Location

### Assembly Bos_taurus_UMD_3.1.1

chr15:44,835,477-44,836,082

### Annotation

### Repeats

### Name: Bov-tA3

Family: tRNA-Core-RTE

### Class: SINE

### Name: BovB

### Family: RTE-BovB

### Class: LINE

### Sequence JC89 (Under-Represented)

(SEQ ID NO: 148)

GTATAGATGTGAGAGTTGGACTGTGAAGAAGGCTGAGTGCTGAAGAATTG
ATGCTTTTGAACTGTGGTGTTGGAGAAGACTCTTGAGAGTCCCTTGGACT
GCAAGGAGATCCAACCAGTCCATTCTAAAGGAGGTCAGTCCTGGGTGTTC
ATTGGAAGGAATGATGCTAAAGCTGAAACTCCAGTACTTTGGCCACACAA
AAAACCAAACA

### Location

### Assembly Bos_taurus_UMD_3.1.1

chr13:33,526,513-33,526,708

### Annotation

### Repeats

### Name: BovB

### Family: RTE-BovB

### Class: LINE

### Sequence JC90 (Under-Represented)

(SEQ ID NO: 149)

GATAGCATATTAAAAATCAGAGACATTACTTTGCTGACAAAGGTTCATCT
AGTTAAGGCTATGGTTTTTCCAGTAGTCATGTATGGAATTGAGAATTGCA
GTATAAAGAAAGCTGAGCACCGAAGAATTGATGCTTTTGAACTGTGGTGT
TGGAGAAGACTCTTGAGAGTCCCTTGGACTGCAAGGAGATCCAACCAGTC
CATTCTGAAGGAGATCAGCCCTGGGATTTCTTTGGGAG

### Location

### Assembly Bos_taurus_UMD_3.1.1

chr29:20,418,647-20,418,884

### Annotation

### Genes

### RefSeq: NM_001102336.1

Description: Bos taurus leucine zipper protein 2 (LUZP2), mRNA.

### Gene Symbol: LUZP2

### Repeats

### Name: BovB

### Family: RTE-BovB

### Class: LINE

### Sequence JC91 (Under-Represented)

(SEQ ID NO: 150)

CAGGACTGGAAAAGGTCAGTTTTCATTCCAATCCCAAAGAAAGGCAATGC
CAAAGAATGCTCAAACTACCGCACAATTGTACTCATCTCACATGCTAGTA
AAGTAATGCTCAAAATTCTCCAGGCCAGGCTTCAGCAATATGTGAACCAT
GAACTTCCTGATGTTAAAGGCAGAAGAACCAGAGATCAAATTGCCAACAT
CCACTGGATCATAAAAAAAGCAAGAGAGTTCCAGAAAAGCATC

### Location

### Assembly Bos_taurus_UMD_3.1.1

chr15:49,580,120-49,580,362

### Annotation

### Repeats

### Name: BovB

### Family: RTE-BovB

### Class: LINE

### Sequence JC92 (Under-Represented)

(SEQ ID NO: 151)
GTGCAATTGTGCGGTAGTTTGAGCATTCTTTGGCATTGCCTTTCTTTGGG
ATTGGAATGAAAACTGACCTTTTCCAGTCCTGTGGCCACTGCTGAGTTTC
CAAATTTGCTGGCATATTGAGTGCAGCACTTTCATAGCATCATCTTTTAG
GATTTGAAATAGCTCACCTGGAACTCCATCACCTCCGCTACCTTTGTTTG
TAGTGATGCTTCCTAAAGCCCACTTGACTTCACATTCCAGGATGTCTGGA
TCTAGGTGAG

### Location

### Assembly Bos_taurus_UMD_3.1.1

chr2:103,004,793-103,005,045

### Annotation

### Repeats

### Name: BTLTR1

### Family: ERVK

### Class: LTR

### Name: BovB

### Family: RTE-BovB

### Class: LINE

### Sequence JC93 (Under-Represented)

(SEQ ID NO: 152)
CAAATATATTGAGTCTTTTAACCACCATCTCCATAAATGGCATTAAATCC
ACCAGTATTGGATTCCAGCTGAGTTATTTCAAATCCTAGAAGATGATGCT
GTGAAACTGCTGCACTCAATATGCCAGCAAATTTGGAAAACTCAGCAGTG
GCCACAGGACTGGAAAAGGTCAGTTTTCATTCCAATCCCAAAGAAAGGCA
ATGCCAAAGAATGCTCAAACTACTGCACAATTGCACT

### Location

### Assembly Bos_taurus_UMD_3.1.1

chr2:3,084,175-3,084,409

### Annotation

### Repeats

### Name: BTLTR1

### Family: ERVK

### Class: LTR

### Name: BovB

### Family: RTE-BovB

### Class: LINE

### Sequence JC94 (Under-Represented)

(SEQ ID NO: 153)
GATCTCCTTTAGGATGGACTGGTTGGATCTCCTTGCAGAACAAGGGACTC
TCGAGTCTTTTCCAACACCGCAGTTCAAAAGCATCAATTCTTTGGCACTC
AGCTTTCTTTATAGTTCAACTCTCACATCCATACATGACCACTGGAAAAA
CCATAGCCTTGACTAGACGGACCTTTATTGACAAAGTAATGTCTCTGCTT
TTGAATATGCTGTCTAGGTTGGTCATAACTTTCCTTCCAAGGAGTAAGTG
TCTTTTAATTTCATGGCTGCAGTCACCATCTGCAGTGAT

### Location

### Assembly Bos_taurus_UMD_3.1.1

chr8:26,316,238-26,316,595

### Annotation

### Repeats

### Name: BovB

### Family: RTE-BovB

### Class: LINE

### Sequence JC95 (Under-Represented)

(SEQ ID NO: 154)
ATCCAACGGATGTTGGCAATTTGATCTCTGGTTCCTCTGCCTTTTCTAAA
GCCAGCTTGAACATCTGGAAGTTCAGATTCACGTACTGCTGAAGCCTGGC
TTGGAGAATTCTGAGCATTACTTTACTAGCGTGTGAAGTGAATGCAATTG
TGCGGTAGTTTGAGCATTCTTTGGCATTGCCTTTCTTTGGGATTGGAATG
AAAACTGACCTTTTCCAGTCCTGTGGCCACTGCTGAGTTTTCCAAATTTG
CTGGCATATTGAGTGC

### Location

### Assembly Bos_taurus_UMD_3.1.1

chr29:27,463,561-27,463,826

### Annotation

### Repeats

### Name: BovB

### Family: RTE-BovB

### Class: LINE

### Sequence JC96 (Under-Represented)

(SEQ ID NO: 155)
GCATCATCTTTCAGGATTTGGAATAGGTCAGTTTTCATTCCAATCCCAAA
GAAAGGCAATGCCAAAGAATGCTCAAACTACTGCACAATTGCACTCATCT
CACACGCTAGTAAAGTAATGCTCAAAATTCTCCAAGCCAGGCTTCAGCAA
TATGTGAACCGTGAACTTCCTGATGTTCAAGCTGGCAGGGGAACCAGAGA
TCAAATTGCCAACATCCGCTGGATCATGGAAAAAGCAGAGAGTTCCAGAA
AAACATCTATTTC

### Location

### Assembly Bos_taurus_UMD_3.1.1

chr17:56,918,088-56,918,338

### Annotation

### Genes

### RefSeq: NM_001075763.2

Description: Bos taurus coiled-coil domain containing 63 (CCDC63), mRNA.

### Gene Symbol: CCDC63

### Repeats

### Name: BovB

### Family: RTE-BovB

### Class: LINE

### Sequence JC98 (Under-Represented)

(SEQ ID NO: 156)
AGTACAGCCACTATGGAGAACAGCGTGGAGATTCCTTAAAAAACTGGAAA
TAGAACTGCCTTATGATCCAGCAATCCCACTGCTGGGCATACACACTGAG
GAAACCAGAAGGGAAAGAGACACGTGTACCCCAATGTTCATTGCAGCACT
GTTCCAGGACATGGAAGCAACCTAGATGTCCATCAGCAGATGAATGGATA
AGAAAGCTGTGGTACATATATACAA

### Location

### Assembly Bos_taurus_UMD_3.1.1

chr2:34,160,472-34,160,696

### Annotation

### Repeats

### Name: L1 BT

### Family: L1

### Class: LINE

### Sequence S49 (Over-Represented)

(SEQ ID NO: 157)
GTACCCATTTTTTACTAGAGAGCGAGATTACTGGCTTGACTGCTCTCTCC
CCCTTTGGACTCTCCTTTTTCTCCACCAGGTCGTCTGTGTCTCCTCCCTA
ACCCCTCTCTACTTTACCCAACTCTGTGAATTTCTGTGTGTTCCAGATGG
TGGGGAACACTTAGGGAACTGATTACTGGCTGGATCTGTCTCCCTTCTTT

### Location

### Assembly Bos_taurus_UMD_3.1.1

chr10:22,785,125-22,785,324

### Annotation

### Repeats

### Name: L1 BT

### Family: L1

### Class: LINE

### Sequence S53 (Over-Represented)

(SEQ ID NO: 158)
ATCGCAGCACTGTTTATAATAGCCAGGACATGGAAGCAACCTAGATGTCC
ATCAGCAGATGAATGGATAAGAAAGCTGTGGTACATATACACAATGGAGT
ATTACTCAGCCATTAAAAAGAATACATTTGAATCAGTTCTAATGAGGTGG
ATGAAACTGGAGCCTATTATACAGAGTGAAGTAAGCCAGAAAAAAAAAAC

### Location

### Assembly Bos_taurus_UMD_3.1.1

chr24:29,814,601-29,814,800

### Annotation

### Repeats

### Name: L1 BT

### Family: L1

### Class: LINE

### Name: BTLTR1

### Family: ERVK

### Class: LTR

### Sequence S54 (Over-Represented)

(SEQ ID NO: 159)
TTTTCCATGCAAATAGGGACCAAAAGAAAGCAGGAGTAGCAATACTCATA
TCAGATAAAATAGACTTTAAAACAAAGGCTGTGAAAAGAGACAAAGAAGG
TCACTACATAATGATTAAAGGATCAATCCAAGAAGAAGATATAACAATTA
TAAATATATATGCACCCAACACGGGAGCACCGCAGTATGTAAGACAAATG

### Location

### Assembly Bos_taurus_UMD_3.1.1

chr1:22,612,199-22,612,398

### Annotation

### Repeats

### Name: L1 BT

### Family: L1

### Class: LINE

### Sequence S55 (Over-Represented)

(SEQ ID NO: 160)
CTAGGGATCTCTTCAAGGAAATCAGAGATACCAAAGGAAGATTTCATGCA
AAGATGAGCTCGATAAAGGACAAAAATGGTATGGACCTAACAGACGCAGA
ATATATTAAGAAGAGATGGCAACAATACACAGAAGAATCATACAAAAAAG
GTCTTCACGACCCAGATAATCACGATGGTGTGATCACTGACCTAGAGCCA

### Location

### Assembly Bos_taurus_UMD_3.1.1

chr10:99,151,264-99,151,463

### Annotation

### Repeats

### Name: BovB

### Family: RTE-BovB

### Class: LINE

### Sequence SC3 (Under-Represented)

(SEQ ID NO: 161)
GCATCGTTCGGAAAAAACCCCAGGTTCCAAATACAGCTCGACAAGCGGCC
TCTCTCCCCGGGGACATCTCGAGAGGCAAGCGGAGCTCCATGCCTCAACC
CAAGACGAGGCCTGACTCTCCTGTCCCCAGTCTGCAGGGACCCTGCGATC
GGAGTCTGAAATCAGAGGTACCCTGCGGTTCCTGCCTCAACTGGAGATGA

### Location

### Assembly Bos_taurus_UMD_3.1.1

chr3:82,757,239-82,757,438

### Annotation

### Repeats

### Name: BTSAT4

Family: centr

### Class: Satellite

### Sequence SC5 (Under-Represented)

(SEQ ID NO: 162)
AGGTGGGAGGGGCCTCTCGGGACTTTTGGGGGTTTGGTGCATTGGAAGAG
GGCCTCATCTCCAGTTGAGGCAGGAACCGCAGGGTACCTCTGATTTCAGA
CTCCGATCGCAGGGTCCCTGCAGACTGGGGACGGGAGAGTCAGGCCTCGT
CTTGGGTTGAGGCATGGGACTCCGCTTGCCTCTCGAGATGTCCCCGGGGA

### Location

### Assembly Bos_taurus_UMD_3.1.1

chr1:45,727,925-45,728,124

### Annotation

### Repeats

### Name: BTSAT4

Family: centr

### Class: Satellite

### Sequence SC6 (Under-Represented)

((SEQ ID NO: 163)
TGTTTTTATCTCACGTAATGCCATAACAAACCACTTGTGGAATCTTTGTT
CCTGACTAGAGATCAAGCCCTGAACCTTTGGAGTGGGAGCACTGACTCCA
AGACTATAGCCTACCAGAGAGCTAACCCCAGGGAGTATCAAATAGCGGGA
ACTCACACAAAGGAAAGCACTTGAAAAGGACCCAGCGTCACCCGCCGCCA

### Location

### Assembly Bos_taurus_UMD_3.1.1

chr24:62,416,051-62,416,250

### Annotation

### Repeats

### Name: L1-3 BT

### Family: L1

### Class: LINE

### Sequence SC7 (Under-Represented)

(SEQ ID NO: 164)
TCAACACGAAGGGGCAGTGACCCGCCCGTGCATCGTCCGGAAAAGACCCC
CAGGTTCCAAATACAGCTCGACAAGTGGCCTCTCTCCCCGGGGACACCTC
GAGAGGCAAGCGGAGTTCCATGCCTCAACCCAAGACGAGGCCTGACTCTC
CTGTCCCCAGTCTGCAGGGACCCTGCGATCGGAGTCTGAAATCAGAGGAA

### Location

### Assembly Bos_taurus_UMD_3.1.1

chr9:45,016,883-45,017,082

### Annotation

### Repeats

### Name: BTSAT4

Family: centr

### Class: Satellite

All publications mentioned herein are hereby incorporated by reference in their entireties. While the foregoing invention has been described in some detail for purposes of clarity and understanding, it will be appreciated by one skilled in the art, from a reading of the disclosure, that various changes in form and detail can be made without departing from the true scope of the invention in the appended claims.

Specific examples of methods and kits have been described herein for purposes of illustration. These are only examples. The technology provided herein can be applied to systems other than the example systems described above. Many alterations, modifications, additions, omissions, and permutations are possible within the practice of this invention. This invention includes variations on described embodiments that would be apparent to the skilled addressee, including variations obtained by: replacing features, elements and/or acts with equivalent features, elements and/or acts; mixing and matching of features, elements and/or acts from different embodiments; combining features, elements and/or acts from embodiments as described herein with features, elements and/or acts of other technology; and/or omitting combining features, elements and/or acts from described embodiments.

The embodiments of the invention described above are intended to be exemplary only. Those skilled in this art will understand that various modifications of detail may be made to these embodiments, all of which come within the scope of the invention.

### APPENDIX

TABLE 1

| Marker ID | Marker Name (internal) | Assembly | Genomic Region | Sequence | fwd primer sequence | rev primer sequence |
|---|---|---|---|---|---|---|
| bov-jd-0001 | J6 | UMD 3.1.1 | Chromosome | TACCATGGTGACCACGGGTGACGGGGAAT | GGAATCAGGGTT | GGTCGGGA |
| | | | 25: | CAGGGTTCGATTCCGGAGAGGGAGCCTGA | CGATTCCG (SEQ ID | GTGGGTAA |
| | | | 32377622-32377759 | GAAACGGCTACCACATCCAAGGAAGGCAG | NO: 64) | TTTG (SEQ |
| | | | | CAGGCGCGCAAATTACCCACTCCCGACCC | | ID NO: 65) |
| | | | | GGGGAGGTAGTGACGAAAAATA (SEQ ID | | |
| | | | | NO: 1) | | |
| bov-jd-0002 | J7 | UMD 3.1.1 | Chromosome | GCTATCCTGAGGGAAACTTCGGAGGGAAC | GGTCGGACGACC | ACCCGAAA |
| | | | 18: | CAGCTACTAGATGGTTCGATTAGTCTTTCG | GATTTG (SEQ ID | GATGGTGA |
| | | | 58238154-58238329 | CCCCTATACCCAGGTCGGACGACCGATTTG | NO: 66) | ACTATG |
| | | | | CACGTCAGGACCGCTACGGACCTCCACCA | | (SEQ ID NO: |
| | | | | GAGTTTCCTCTGGCTTCGCCCTGCCCAGGC | | 67) |
| | | | | ATAGTTCACCATCTTTCGGGTCCTAACA | | |
| | | | | (SEQ ID NO: 3) | | |
| bov-jd-0003 | J8 | UMD 3.1.1 | Chromosome | TTCGCTGGATAGTAGGTAGGGACAGTGGG | GACAGTGGGAAT | GTAAACGG |
| | | | X: | AATCTCGTTCATCCATTCATGCGCGTCACT | CTCGTTCATC (SEQ | CGGGAGTA |
| | | | 62078561-62078748 | AATTAGATGACGAGGCATTTGGCTACCTTA | ID NO: 68) | ACTATG |
| | | | | AGAGAGTCATAGTTACTCCCGCCGTTTACC | | (SEQ ID NO: |
| | | | | CGCGCTTCATTGAATTTCTTCACTTTGACAT | | 69) |
| | | | | TCAGAGCACTGGGCAGAAATCACATCGCG | | |
| | | | | TCAACACCC (SEQ ID NO: 5) | | |
| bov-jd-0004 | J13 | UMD 3.1.1 | Chromosome | AGATGGATTTAGAGTGTGTGGCTGTTGTG | TCTGAGTAGTCCT | GTAGGTGG |
| | | | 21: | TGTGGTTCAGAATTCTGAGTAGTCCTTTCA | TTCAGGGAT (SEQ | CTCGTATTA |
| | | | 2014523-2014688 | GGGATCACCCAAAAGCTCAGACAAAGCGT | ID NO: 70) | TCTTTCT |
| | | | | GAGCCCTTCACGTGGACTGTGCTGTGCGT | | (SEQ ID NO: |
| | | | | CAGAAAGATAATACGAGCCACCTACACAA | | 71) |
| | | | | TCTAAAATTTCCTAAAAACC (SEQ ID NO: | | |
| | | | | 7) | | |
| bov-jd-0005 | J14 | UMD 3.1.1 | Chromosome | AGCTGAAGTGGTGGGATAGTGTAGTTTGT | GTGGTGGGATAG | CACAGTCTT |
| | | | 1: | GTGTCATAGGAATCTCCTCAAAGGATGAT | TGTAGTTTGT (SEQ | TGAAGGCA |
| | | | 33090510-33090673 | GTCATCAGTGTGAGGTCCTAATTGTGTCCT | ID NO: 72) | ACATT (SEQ |
| | | | | AGAAAAAGTTAGAGCTGGTGAAAATGTTG | | ID NO: 73) |
| | | | | CCTTCAAAGACTGTGTGAGATGGCAGGTG | | |
| | | | | TGGACACTGGCCCCACTG (SEQ ID NO: 9) | | |
| bov-jd-0006 | J16 | UMD 3.1.1 | Chromosome | GGGTGTTGGTTCTCCTTGCAGGTCCTGGA | CTTGCAGGTCCTG | CTCTCTTCT |
| | | | 6: | GATACAGCCAAAGGCCCAGTTTTTGAGAG | GAGATA (SEQ ID | ACATTAGTT |
| | | | 5440642-5440791 | AACTCTTCCTAACTGTCTTCTTGTCTGAAAC | NO: 74) | TCAGACA |
| | | | | TAATGTAGAAGAGAGATTTTCACTAGTAG | | (SEQ ID NO: |
| | | | | TAGTTTAGCTTATTTAGCCTGATTTCCACCA | | 75) |
| | | | | C (SEQ ID NO: 11) | | |
| bov-jd-0007 | J17 | UMD 3.1.1 | Chromosome | GTTGTTTGTAATATTACTTAGGTATTTTTAA | TCATTGCTGTGTG | AGGCTCTG |
| | | | 14: | CATTTTTATTGGAAAGTGAAAGTTAGTTTT | CAGTCT (SEQ ID | CAACAAGA |
| | | | 1093166-1093354 | GGTTGTGCTGGGTTTTCATTGCTGTGTGCA | NO: 76) | GAAG (SEQ |
| | | | | GTCTTTCCCTAGTTGCAGAGTGTGGGGGC | | ID NO: 77) |
| | | | | TACTCTTTGTTTTAGGCTTTTCATTGTGGTG | | |
| | | | | GCTTCTCTTGTTGCAGAGCCTGGGCTGCA | | |
| | | | | GAGTGTGTG (SEQ ID NO: 13) | | |
| bov-jd-0008 | Control 1 | UMD 3.1.1 | Chromosome | GATCTTATTCTTTGAAGTAATGTTATGAAT | TCTTATCGGACAA | TGGTTTCTG |
| | | | 6: | CTTATCGGACAAAGCCAGACTTCTTCATTT | AGCCAGAC (SEQ | TAACCTTCC |
| | | | 5425625-5425755 | GGGAGTAGATTTGGAAGAGAACACTTGGT | ID NO: 78) | TGAG (SEQ |
| | | | | CTCAGCACTCCCCTTCTCAGGAAGGTTA | | ID NO: 79) |
| | | | | CAGAAACCAAAA (SEQ ID NO: 15) | | |
| bov-jd-0009 | Universal | UMD 3.1.1 | Chromosome | GTTTGTAACCTGGGACCCTTGAGTTAATTC | CTCCTGACCAACC | GGCTTTCTT |
| | | JU1 | 1: | TTTTTCTTGTTATAGCCCACCACACCTTTGC | ACCTTTAG (SEQ ID | CCTGCAAA |
| | | | 29318042-29318377 | TCTGTAGGAATGCAACTTTATCTAATGCTT | NO: 80) | CTTATC |
| | | | | TTTTGGAGGGTGGCTCCTGACCAACCACCT | | (SEQ ID NO: |
| | | | | TTAGAGAAAAATAAGTTTTCTGAAGAAAA | | 81) |
| | | | | GGTCTTAAAATGTTAACAGGCCTCCGGGC | | |
| | | | | CAGAAGATGATGCAAATCACCTAAGCTTTT | | |
| | | | | GCATATGATAAGTTTGCAGGAAGAAAGCC | | |
| | | | | TGGTTTGCTGCAAGACTCGACCCCTTCCCC | | |
| | | | | CATTATCCTCTATGCATAACTTAAGGTATA | | |
| | | | | AAAACTACTTTGAAAAATAAAGTGCGGGC | | |
| | | | | CTTGTTCAC (SEQ ID NO: 17) | | |
| bov-jd-0010 | Universal | UMD 3.1.1 | Chromosome | GTGTCCCTGGCAGTGTATTGATTAATATAA | GTCCCTGGCAGTG | CAAAGGTG |
| | JU2 | | 7: | TTGGTGTAAGTAGTAGCTTTAATGTTTGTA | TATTGATT (SEQ ID | TGGTGGGC |
| | | | 34812863-34813185 | ACCTGGGACCCTTGAGTTAATTCTTTTTCTT | NO: 82) | TATAA (SEQ |
| | | | | GTTATAGCCCACCACACCTTTGCTCTGTAG | | ID NO: 83) |
| | | | | GAATGCAACTTTATCTAATGCTTTTTTGGA | | |
| | | | | GGGTGGCTCCTGACCAACCACCTTTAGAG | | |
| | | | | AAAAATAAGTTTTCTGAAGAAAAGGTCTT | | |
| | | | | AAAATGTTAACAGGCCTCCGGGCCAGAAG | | |
| | | | | ATGATGCAAATCACCTAAGCTTTTGCATAT | | |
| | | | | GATAAGTTTGCAGGAAGAAAGCCTGGTTT | | |
| | | | | GCTGCAAGACTCGACCCCTTCCCCCA (SEQ | | |
| | | | | ID NO: 19) | | |
| bov-jd-0011 | Universal | UMD 3.1.1 | Chromosome | ACTACTTACACCAATTATATTAATCAATAC | GTTTAGAACATCT | ATCCTCTAG |
| | JU3 | | 3: | ACTGCCAGGGACACAGCAGGTAAGGGAT | CCTGCCTCTC (SEQ | CCTGCCTGA |
| | | | 66900607-66900863 | ATGGAAACTTAGCAGCAAACAATGGCCCA | ID NO: 84) | ATA (SEQ ID |
| | | | | ACAAGTGAAAAACCATTCACCAATACAATT | | NO: 85) |
| | | | | TCTAATCAATCTTGTAACTACTCAAAAGAA | | |
| | | | | TCTGTGTTTAGACAGTTTAGAACATCTCCT | | |
| | | | | GCCTCTCACAGTTGGGAGGCTCTGAACAA | | |
| | | | | TCCCATGTGGCCGGAAAAACCTATTCAGG | | |
| | | | | CAGGCTAGAGGATTTCCAAAGG (SEQ ID | | |
| | | | | NO: 21) | | |
| bov-jd-0012 | Universal | UMD 3.1.1 | Chromosome | CATCATCTTCTGGCCCGGAGGCCTGTTAAC | CAAAGGTGTGGT | GTCCCTGG |
| | JU4 | | 4: | ATTTTAAGACCTTTTCTTCAGAAAACTTATT | GGGCTATAA (SEQ | CAGTGTATT |
| | | | 12626489-1262.6892 | TTTCTCTAAAGGTGGTTGGTCAGGAGCCA | ID NO: 86) | GATT (SEQ |
| | | | | CCCTCCAAAAAAGCATTAGATAAAGTTGC | | ID NO: 87) |
| | | | | ATTCCTACAGAGCAAAGGTGTGGTGGGCT | | |
| | | | | ATAACAAGAAAAAGAATTAACTCAAGGGT | | |
| | | | | CCCAGGTTACAAACATTAAAGCTACTACTT | | |
| | | | | ACACCAATTATATTAATCAATACACTGCCA | | |
| | | | | GGGACACAGCAGGTAAGGGATATGGAAA | | |
| | | | | CTTAGCAGCAAACAATGGCCCAACAAGTG | | |
| | | | | AAAAACCATTCACCAATACAATTTCTAATC | | |
| | | | | AATCTTGTAACTCCTCAAAAGAATCTGTGT | | |
| | | | | TTAGACAGTTTAGAACATCTCCTGCCTCTC | | |
| | | | | ACAGTTGGGAGGCTCTGAAC (SEQ ID NO: | | |
| | | | | 23) | | |
| bov-jd-0013 | S13 | Btau 4.6.1 | Chromosome | TCGCTGGAGGTTGCTAGGACCTCTGGACT | GCTTGTTAGTTCC | CTCTAGCCA |
| | | | 6: | GCAGTCTCCCATCTCTTGCTGACCTCTTGTT | TTGTTCCTTAC | CCTTTGGG |
| | | | 6320256-6320455 | GACCTCTGCAGAATCCTGGTTGCTGGGAG | (SEQ ID NO: 88) | ATG (SEQ ID |
| | | | | CTTGTTAGTTCCTTGTTCCTTACCCGGACCA | | NO: 89) |
| | | | | CCTGTGGTTAAGTGTTTTCTCTCCTCTGTG | | |
| | | | | GCCTGGTAAGGCTGGGTGGTCATTGGCAT | | |
| | | | | CCCAAAGGTGGCTAGAGGTCA (SEQ ID | | |
| | | | | NO: 57) | | |
| bov-jd-0014 | S14 | Btau 4.6.1 | Chromosome | CATATCTATTCACAAGCCTGCCTAAATCAT | CGGGTAAGGAAC | CATCTCTTG |
| | | | 14: | TGACCTCTAGCCACCTTTGGGATGCCAATG | AAGGAACTAA | CTGACCTCT |
| | | | 53324225-53324424 | ACCACCCAGCCTTACCAGGCCACAGAGGA | (SEQ ID NO: 90) | TGT (SEQ ID |
| | | | | GAGAAAACACTTAACCACAGGTGGTCCGG | | NO: 91) |
| | | | | GTAAGGAACAAGGAACTAACAAGCTCCCA | | |
| | | | | GCAACCAGGATTCTGCAGAGGTCAACAAG | | |
| | | | | AGGTCAGCAAGAGATGGGAGACTG (SEQ | | |
| | | | | ID NO: 58) | | |
| bov-jd-0015 | S16 | Btau 4.6.1 | Chromosome | GAAACAGGAAGCCCAGAGGTGCCCCTGTA | CCTTAGGAGGTCA | GAACATCC |
| | | | 6: | GTTTGGCGAAGGCCTTAGGAGGTCACTTG | CTTGGAAAT (SEQ | ATGCAAAG |
| | | | 6338951-6339151 | GAAATGCCGCTGTCTGTGAGGGGGGGAG | ID NO: 92) | GACAAG |
| | | | | GGGGCACATGTGCACAGAGCTGTGCAGG | (SEQ ID NO: | |
| | | | | CTTGTCCTTTGCATGGATGTTCTTTGTAGA | 93) | |
| | | | | AATCCACCTCGGAGCCATCTCCTGATGTGT | | |
| | | | | AAGAATTTCCCAGACAAACCTTGAGCT | | |
| | | | | (SEQ ID NO: 59) | | |
| bov-jd-0016 | S17 | Btau 4.6.1 | Chromosome | GGGTGTGTGTGTGTGGGGGGGGGAGCCC | GAGCCCTTCTTTC | TTCACCCAC |
| | | | 7: | TTCTTTCCATTCCACATGAGACCCAGTGGA | CATTCCA (SEQ ID | ATTACCGTT |
| | | | 62628006-62628205 | CAATCCAGGCCCTGCCCACTGCATTGAGTG | NO: 94) | GATA (SEQ |
| | | | | TGCAGCAACAATATCAACGGTAATGTGGG | | ID NO: 95) |
| | | | | TGAACAACATGTATTCCTGGAATCCACTCC | | |
| | | | | GTTGGCTACTGACTGTGTGTGGTGGTGGT | | |
| | | | | CTGGAACCTAGACCTGGAAGCTTT (SEQ ID | | |
| | | | | NO: 60) | | |
| bov-jd-0017 | S18 | Btau 4.6.1 | Chromosome | GTTTATCTAAAAGCTCATATCACATTTAATT | CCTGTTCTTTCGA | CATAGTGC |
| | | | 26: | TTTCGTTTTGTTCCTGTTCTTTCGAGGTACT | GGTACTTTCT (SEQ | CTCGGTTTG |
| | | | 15253287-15253486 | TTCTTGTTGACAAGCTTGTGACAGATAGAA | ID NO: 96) | TTCTA (SEQ |
| | | | | CGATATAGCAATTTTTACCTTAGAACAAAC | | ID NO: 97) |
| | | | | CGAGGCACTATGAACATTTTGTGCTTCATG | | |
| | | | | TTGATGACTCTTAGACATGTCTACAGTAGA | | |
| | | | | GGAGCAAAAACAAAACTA (SEQ ID NO: | | |
| | | | | 61) | | |
| bov-jd-0018 | S19 | Btau 4.6.1 | Chromosome | GATACTCGCCAGCTGAGCCGCGCGACACC | ACATCTGCCCGCT | CTCAGCTG |
| | | | 6: | TGCATGCTCATCTGCCTGATACTCGCCAGC | CATC (SEQ ID NO: | GCGAGTAT |
| | | | 76185750-76186050 | TGAGCCGCGCGACATCTGCCCGCTCATCA | 98) | CAG (SEQ ID |
| | | | | GCCTGATACTCGCCAGCTGAGCCCCGCGA | | NO: 99) |
| | | | | CACCTGCCCGCTCATCAGCCTGATACTCGC | | |
| | | | | CAGCTGAGCCCCGCGACACCTGCCCGCTC | | |
| | | | | ATCTGCCTGATACTCGCCAGCTGAGCCGC | | |
| | | | | GCGACACCTGCCCGCTCATCTGCCTGATAC | | |
| | | | | TCGCCAGCTGAGCCGCGCGACACCTGCCC | | |
| | | | | GCTCATCTGCCTGATACTCTCCAGGTGAAT | | |
| | | | | CGCGCCA (SEQ ID NO: 62) | | |
| bov-jd-0019 | S20 | Btau 4.6.1 | Chromosome | CGACCGGAAGGTCGGGAACCCCTTGCAGA | AAACCTCAGGGTT | CCACAATTG |
| | | | 25: | CAAAGCAGGGGAGTCGACCCTCCCGTCCA | CCTCTC (SEQ ID | GAGGTCGA |
| | | | 37331550-37331750 | GATCAGGAGGGGAGAAAGGGCTCAGAGG | NO: 100) | AAG (SEQ ID |
| | | | | AGGGGGTGCCGGAAAACCTCAGGGTTCCT | | NO: 101) |
| | | | | CTCGAGGGAGACCGGGATTTCGGGGAACT | | |
| | | | | TTGTGGGTCGCATCAAGGGTGCCAAGTGC | | |
| | | | | CCTTTCGACCTCCAATTGTGGCTTCTTG | | |
| | | | | (SEQ ID NO: 63) | | |
| bov-jd-0020 | Universal | UMD 3.1.1 | Chromosome | ACCAAGTTTCGGTGAACAAGGCCCGCACT | AACAAGGCCCGC | GCATATGA |
| | JU5 | | 4: | TTATTTTTCAAAGTAGTTTTTATACCTTAAG | ACTTTA (SEQ ID | TAAGTTTGC |
| | | | 51996600-51996918 | TTATGCATAGAGGATAATGGGGGAAGGG | NO: 102) | AGGAAGAA |
| | | | | GTCGAGTCTTGCAGCAAACCAGGCTTTCTT | | (SEQ ID NO: |
| | | | | CCTGCAAACTTATCATATGCAAAAGCTTAG | | 103) |
| | | | | GTGATTTGCATCATCTTCTGGCCCGGAGGC | | |
| | | | | CTGTTAACATTTTAAGACCTTTTCTTCAGAA | | |
| | | | | AACTTATTTTTCTCTAAAGGTGGTTGGTCA | | |
| | | | | GGAGCCACCCTCCAAAAAAGCATTAGATA | | |
| | | | | AAGTTGCATTCCTACAGAGCAAAGGTGTG | | |
| | | | | GTGGGCTATAACAAGAAAAAGA (SEQ ID | | |
| | | | | NO: 25) | | |
| bov-jd-0021 | Universal | UMD 3.1.1 | Chromosome | ACACCTTTGCTCTGTAGGAATGCAACTTTA | GCCAGAAGATGA | CTTAAGTTA |
| | JU6 | | 25: | TCTAATGCTTTTTTGGAGGGTGGCTCCTGA | TGCAAATCAC | TGCATAGA |
| | | | 36122680-36122976 | CCAACCACCTTTAGAGAAAAATAAGTTTTC | (SEQ ID NO: 104) | GGATAATG |
| | | | | TGAAGAAAAGGTCTTAAAATGTTAACAGG | | GG (SEQ ID |
| | | | | CCTCCGGGCCAGAAGATGATGCAAATCAC | | NO: 105) |
| | | | | CTAAGCTTTTGCATATGATAAGTTTGCAGG | | |
| | | | | AAGAAAGCCTGGTTTGCTGCAAGACTCGA | | |
| | | | | CCCCTTCCCCCATTATCCTCTATGCATAACT | | |
| | | | | TAAGGTATAAAAACTACTTTGAAAAATAA | | |
| | | | | AGTGCGGGCCTTGTTCACCGAAACTTGGT | | |
| | | | | C (SEQ ID NO: 27) | | |
| bov-jd-0022 | Universal | UMD 3.1.1 | Chromosome | TGCATAGAGGATAATGGGGGAAGGGGTC | GCTTAGGTGATTT | TTTGGAGG |
| | JU7 | | X: | GAGTCTTGCAGCAAACCAGGCTTTCTTCCT | GCATCATCTTC | GTGGCTCCT |
| | | | 30978436-30978636 | GCAAACTTATCATATGCAAAAGCTTAGGT | (SEQ ID NO: 106) | (SEQ ID NO: |
| | | | | GATTTGCATCATCTTCTGGCCCGGAGGCCT | | 107) |
| | | | | GTTAACATTTTAAGACCTTTTCTTCAGAAA | | |
| | | | | ACTTATTTTTCTCTAAAGGTGGTTGGTCAG | | |
| | | | | GAGCCACCCTCCAAAAAAGCATTA (SEQ ID | | |
| | | | | NO: 29) | | |
| bov-jd-0023 | Universal | UMD 3.1.1 | Chromosome | GAAATCCTCTAGCCTGCCTGAATAGGTTTT | GTCCCTGGCAGTG | CAAAGGTG |
| | JU8 | | 28: | TCCGGCCACATGGGATTGTTCAGAGCCTCC | TATTGATT (SEQ ID | TGGTGGGC |
| | | | 40139956-40140405 | CAACTGTGAGAGGCAGGAGATGTTCTAAA | NO: 108) | TATAA (SEQ |
| | | | | CTGTCTAAACACAGATTCTTTTGAGTAGTT | | ID NO: 109) |
| | | | | ACAAGATTGATTAGAAATTGTATTGGTGA | | |
| | | | | ATGGTTTTTCACTTCTTGGGCCATTGTTTGC | | |
| | | | | TGCTAAGTTTCCATATCCCTTACCTGCTGT | | |
| | | | | GTCCCTGGCAGTGTATTGATTAATATAATT | | |
| | | | | GGTGTAAGTAGTAGCTTTAATGTTTGTAAC | | |
| | | | | CTGGGACCCTTGAGTTAATTCTTTTTCTTGT | | |
| | | | | TATAGCCCACCACACCTTTGCTCTGTAGGA | | |
| | | | | ATGCAACTTTATCTAATGCTTTTTTGGAGG | | |
| | | | | GTGGCTCCTGACCAACCACCTTTAGAGAA | | |
| | | | | AAATAAGTTTTCTGAAGAAAAGGTCTTAA | | |
| | | | | AATGTTAACAGGCCTCCGGGCCAGAAGAT | | |
| | | | | GAT (SEQ ID NO: 31) | | |
| bov-jd-0024 | Universal | UMD 3.1.1 | Chromosome | ACCTTAGAACAAACCGAGGCACTATGAAC | ATTGACTAGTTCC | AGCCCTTGC |
| | SU1 | | 8: | ATTTTGTGCTTCATGTTGATGACTCTTAGA | CAGTTCACG (SEQ | AAACCCAA |
| | | | 61277875-61278074 | CATGTCTACAGTAGAGGAGCAAAAACAAA | ID NO: 110) | (SEQ ID NO: |
| | | | | ACTACTAGATATTTCATATTGACTAGTTCCC | | 111) |
| | | | | AGTTCACGGGACTCTGACATTCCCTGAGGT | | |
| | | | | CAAAGTTTTCTTGTATTGGAAGCAGTTGGG | | |
| | | | | TTTGCAAGGGCTGCCTTGTCT (SEQ ID NO: | | |
| | | | | 33) | | |
| bov-jd-0025 | Universal | UMD 3.1.1 | Chromosome | AGAAAACTTTGACCTCAGGGAATGTCAGA | GAGTCATCAACAT | AGCTTGTG |
| | SU2 | | 6: | GTCCCGTGAACTGGGAACTAGTCAATATG | GAAGCACAA (SEQ | ACAGATAG |
| | | | 19804114-19804313 | AAATATCTAGTAGTTTTGTTTTTGCTCCTCT | ID NO: 112) | AACGATA |
| | | | | ACTGTAGACATGTCTAAGAGTCATCAACAT | | (SEQ ID NO: |
| | | | | GAAGCACAAAATGTTCATAGTGCCTCGGT | | 113) |
| | | | | TTGTTCTAAGGTAAAAATTGCTATTGCTAT | | |
| | | | | ATCGTTCTATCTGTCACAAGCT (SEQ ID | | |
| | | | | NO: 35) | | |
| bov-jd-0026 | Universal | UMD 3.1.1 | Chromosome | TGGACCCCAGAAAAGAGTGTTGTGCCTAG | CTTCCGTGATCTC | GTCAGGTA |
| | SU3 | | 27: | CACAAGGTTTCTTGAGAGGTTGAACTGCCT | TTCTAGGTTG | GGATCACA |
| | | | 26332496-26332695 | TTGCTAATGGATTTTAAGTTTCTTTACCTCT | (SEQ ID NO: 114) | CAAGTC |
| | | | | TCCGTGATCTCTTCTAGGTTGACCTAGAAG | | (SEQ ID NO: |
| | | | | AGAAATCTTTTGTTAGCTTTGGCTAAGTGG | | 115) |
| | | | | AGAAGTATTGCTTCACGGTGACTTGTGTG | | |
| | | | | ATCCTACCTGACTGTTCTAAA (SEQ ID NO: | | |
| | | | | 37) | | |
| bov-jd-0027 | Universal | UMD 3.1.1 | Chromosome | CAGAAAAGAATGTTGTGCCTAGCACAAGG | AGGTTGACCTAGA | GGGTTTAG |
| | SU4 | | 28: | TTTCTTGAGAGGTTGAACTGCCTTTGCTAA | ACAGAAATCTT | AACAGTCA |
| | | | 30011111-30011310 | TGGATTTTAAGTTTCTTTACCTCTTCCGTGA | (SEQ ID NO: 116) | GGTAGG |
| | | | | TCTCTTCTAGGTTGACCTAGAACAGAAATC | | (SEQ ID NO: |
| | | | | TTTTGTTAGCTTTGGCTAAGTGGAGAAGTA | | 117) |
| | | | | TTGCTTCACGGTGACTTGTGTGATCCTACC | | |
| | | | | TGACTGTTCTAAACCCTTTT (SEQ ID NO: 39) | | |
| bov-jd-0028 | Universal | UMD 3.1.1 | Chromosome | GTATCCTGGCACATACATGTACTGCCTC | GACTATGCTGGGT | TTCTGCAGA |
| | SU5 | | 4: | ACCCCTAACCTCAGTCATGTGTTCCAAGA | CTTTGCT (SEQ ID | GGTCAACA |
| | | | 103646990-103647189 | ACTTCTGTCCGTTTATTTATTTGTTTTTGG | NO: 118) | AGAG (SEQ |
| | | | | TAACATTGAATTCGTTTTGACTATGCTGG | | ID NO: 119) |
| | | | | GTCTTTGCTGCTCGCTGGAAGTTGCTAGG | | |
| | | | | ACCTCTGGACTGCAGTCTCCCATCTCTTG | | |
| | | | | CTGACCTCTTGTTGACCTCTGCAGAA (SEQ | | |
| | | | | ID NO: 41) | | |
| bov-jd-0029 | Universal | UMD 3.1.1 | Chromosome | ACAAGAAAACTTTGACCTCAGGGAATGTC | ACTTTGACCTCAG | TGCTTCGTG |
| | SU6 | | 27: | AGAGTCCCGTGAACTGGGAACTAGTCAAT | GGAATGTC (SEQ | TTGATGACT |
| | | | 23691681-23691880 | ATGAAATATCTAGTAGTTTTGTTTTTGCTCC | ID NO: 120) | CTTA (SEQ |
| | | | | TCTACTGTAGACATGTCTAAGAGTCATCAA | | ID NO: 121) |
| | | | | CACGAAGCACAAAATGTTCATAGTGCCTC | | |
| | | | | GGTTTGTTCTGAGGTAAAAATTGCTATATC | | |
| | | | | GTTCTATCTGTCACAAGCTTGT (SEQ ID | | |
| | | | | NO: 43) | | |
| bov-jd-0030 | Universal | UMD 3.1.1 | Chromosome | AGCCTGCCTAAATCATTGACTTCTAGCCAC | TTGACTTCTAGCC | GCTCGTTA |
| | SU7 | | 27: | CTTTGGGATGCCAATGACCACCCAGCCTTA | ACCTTTGG (SEQ ID | GTTCCTTGT |
| | | | 23723113-23723312 | CCAGGCCACAGAGGAGAGAAAACATTTAC | NO: 122) | TCCT (SEQ |
| | | | | CACATGAGGTCCGGGTAAGGAACAAGGA | | ID NO: 123) |
| | | | | ACTAACGAGCTCCCACCAACCAGGATTCTG | | |
| | | | | CAGAGGTCAACAAGAGGTCAGCAAGAGA | | |
| | | | | TGGGAGACTGCAGTCCAGAGGTCCT (SEQ | | |
| | | | | ID NO: 45) | | |
| bov-jd-0031 | Universal | UMD 3.1.1 | Chromosome | TGACTCTTAGACATGTCTACAGTAGAGGA | ATTGACTAGTTCC | CAAACCCA |
| | SU8 | | 22: | GCAAAAACAAAACTACTAGATATTTCATAT | CAGTTCACG (SEQ | ACTGCTTCC |
| | | | 42434577-42434776 | TGACTAGTTCCCAGTTCACGGGACTCTGAC | ID NO: 124) | AATAC (SEQ |
| | | | | ATTCCCTGAGGTCAAAGTTTTCTTGTATTG | | ID NO: 125) |
| | | | | GAAGCAGTTGGGTTTGCAAGGGCTGCCTT | | |
| | | | | GTCTTGAGACCATTGAACTAAGAACTCAG | | |
| | | | | AACTTGAGCACTATTATCAAAAA (SEQ ID | | |
| | | | | NO: 47) | | |
| bov-jd-0032 | Universal | UMD 3.1.1 | Chromosome | TTGGGTGTCACAGTGGAGGTGTAGAAGA | CTGAGAGTGCAA | TACCTGGCT |
| | SU9 | | 20: 2742-2941 | GGTCTGAGAGTGCAAGGCAGTTAAGGAA | GGCAGTTA (SEQ | ATTTGGTTG |
| | | | | GAAGTACATGGGCTGGGTGATTAGCTGAC | ID NO: 126) | GG (SEQ ID |
| | | | | TGCATGTAATAGAAATCACAATGATCAAAT | | NO: 127) |
| | | | | TCCCCAACCAAATAGCCAGGTAACAGAAT | | |
| | | | | AAGAAAAATAAAAAGCAGAGGATCTGGA | | |
| | | | | CTTTCTTGTCCATAGAAAGTTCTAAGAG | | |
| | | | | (SEQ ID NO: 49) | | |
| bov-jd-0033 | Universal | UMD 3.1.1 | Chromosome | CACCCCTAACCTCAGTCGTGTGTTCCAAGA | CTCGCTGGAAGTT | CTCCCAGCA |
| | SU10 | | X: | ACTTCTGTCCGTTTATTTATTTGTTTTGGGT | GCTAGG (SEQ ID | ACCAGGAT |
| | | | 65494658-65494857 | AACATTGAATTCGTTTTGACTATGCTGGGT | NO: 128) | TC (SEQ ID |
| | | | | CTTTGCTGCTCGCTGGAAGTTGCTAGGACC | | NO: 129) |
| | | | | TCTGGACTGCAGTCTCCCATCTCTTGCTGA | | |
| | | | | CCTCTTGTTGACCTCTGCAGAATCCTGGTT | | |
| | | | | GCTGGGAGCTTGTTAGTTC (SEQ ID NO: 51) | | |
| bov-jd-0034 | Universal | UMD 3.1.1 | Chromosome | CAAGAGGTCAGCAAGAGATGGGAGACTG | CAAGAGGTCAGC | TCCAAGAA |
| | SU11 | | 27: | CAGTCCAGAGGTCCTAGCAACTTCCAGCG | AAGAGATGG (SEQ | CTTCTGTCC |
| | | | 26331117-26331316 | AGCAGCAAAGACCCAGCATAGTCAAAACG | ID NO: 130) | GTTTAT |
| | | | | AATTCAATGTTACCAAAAACAAATAAATAA | | (SEQ ID NO: |
| | | | | ACGGACAGAAGTTCTTGGAACACATGACT | | 131) |
| | | | | GAGGTTAGGGGTGAGGCAGTACATGTAT | | |
| | | | | GTGCCAGGATACTCTTAAAGTATACTC | | |
| | | | | (SEQ ID NO: 53) | | |
| bov-jd-0035 | Universal | UMD 3.1.1 | Chromosome | GACAAGGCAGCCCTTGCAAACCCAACTGC | AGCCCTTGCAAAC | ATTGACTA |
| | SU12 | | 27: | TTCCAATACAAGAAAACTTTGACCTCAGGG | CCAA (SEQ ID NO: | GTTCCCAGT |
| | | | 26341491-26341690 | AATGTCAGAGTCCCGTGAACTGGGAACTA | 132) | TCACG (SEQ |
| | | | | GTCAATATGAAATATCTGGTAGTTTTGTTT | | ID NO: 133) |
| | | | | TTGCTCCTCTACTGTAGACATGTCTAAGAG | | |
| | | | | TCATCAACATGAAGCACAAAATGTTCATAG | | |
| | | | | TGCCTCGGTTTGTTCTAAGGTA (SEQ ID | | |
| | | | | NO: 55) | | |

1. A method for treating a bovine animal suspected of having Johne's disease, the method comprising treating the bovine animal identified as having Johne's disease with a therapeutically effective amount of at least one agent used to treat Johne's disease, wherein the bovine animal is identifiable as having Johne's disease by
analyzing a biological sample isolated from the bovine animal for over-representation or under-representation of at least one polynucleotide relative to an internal standard region, wherein the at least one polynucleotide comprises any one of SEQ ID NOs: 1-16 or 134-164 and wherein the over-representation or under-representation of the at least one polynucleotide in the biological sample is a positive indicator of Johne's disease.

2. The method of claim 1, wherein the at least one polynucleotide comprising any one of SEQ ID NOs: 1-16 or 134-164 over-represented or under-represented relative to the internal standard region is at least two, at least three, at least four, or at least five of the polynucleotides comprising any one of SEQ ID NOs: 1-16 or 134-164.

3. The method of claim 1, wherein the biological sample is blood, a product derived from blood, or a fraction derived from blood.

4. The method of claim 3, wherein the product derived from blood is plasma or serum.

5. The method of claim 1, wherein detecting the over-representation or under-representation of the at least one polynucleotide relative to an internal standard region comprises at least one of a polymerase chain reaction (PCR)-based detection method, a hybridization-based method, enzyme-linked immunosorbent assay (ELISA), radioimmunoassay (MA), solid-phase enzyme immunoassay (EIA), mass spectrometry, and microarray analysis.

6. The method of claim 5, wherein the PCR-based detection method comprises amplifying nucleic acid sequences in the biological sample using primers that are specific for and capable of amplifying any one of SEQ ID NOs: 1-16 or 134-164, wherein the amplifying generates amplification products corresponding to any one of SEQ ID NOs: 1-16 or 134-164 when the biological sample comprises any one of SEQ ID NOs: 1-16 or 134-164.

7. The method of claim 5, wherein the PCR-based detection method is performed using at least one primer pair, wherein each primer pair of the at least one primer pair is specific for any one of SEQ ID NOs: 1-16 or 134-164.

8. The method of claim 7, wherein the primer pair specific for any one of SEQ ID NOs: 1-16 or 134-164 is any one of the primer pairs presented in Table 1.

9. The method of claim 6, further comprising sequencing the amplification products corresponding to any one of SEQ ID NOs: 1-16 or 134-164.

10. The method of claim 6, wherein the nucleic acid sequences comprise circulating nucleic acid.

11. The method of claim 1, wherein the at least one agent used to treat Johne's disease comprises at least one of antibiotic.

12. The method of claim 11, wherein the at least antibiotic is in a genus comprising rifabutin.

13. The method of claim 11, wherein the at least antibiotic is in a genus comprising clarithromycin.

14. The method of claim 11, wherein the at least antibiotic comprises rifabutin and clarithromycin.

15. The method of claim 1, wherein the bovine animal is monitored for Johne's disease.

16. A probe comprising a manmade nucleotide sequence capable of binding specifically to a polynucleotide comprising any one of SEQ ID NOs: 1-16 or 134-164 and at least one manmade tag conjugated thereto, wherein the manmade nucleotide sequence is complementary to the polynucleotide comprising any one of SEQ ID NOs: 1-16 or 134-164.

17. The probe of claim 16, wherein the manmade nucleotide sequence capable of binding specifically to a polynucleotide comprising any one of SEQ ID NOs: 1-16 or 134-164 exhibits at least 90%, at least 91%, at least 92%, at least 93%, at least 94%, or at least 95%, at least 96%, at least 97%, at least 98%, or at least 99% complementarity to any one of SEQ ID NOs: 1-16 or 134-164.

18. The probe of claim 16, wherein the manmade tag is a detectable marker.

19. An array comprising at least one probe comprising a manmade nucleotide sequence capable of binding specifically to a polynucleotide comprising any one of SEQ ID NOs: 1-16 or 134-164, wherein the manmade nucleotide sequence is complementary to the polynucleotide comprising any one of SEQ ID NOs: 1-16 or 134-164, wherein the at least one probe is bound to a solid surface.

20. The array of claim 19, comprising a microarray, gene chip, DNA chip, or a FILMARRAY®.


SEQUENCE LISTING (164 sequences)

SEQ ID NO: 1 - DNA, 138 bp, Bos taurus
taccatggtgaccacgggtgacggggaatcagggttcgattccggagagggagcctgagaaacggctaccacatccaaggaaggcagcaggcgcgcaaattacccactcccgacccggggaggtagtgacgaaaaata

SEQ ID NO: 2 - DNA, 186 bp, Bos taurus
aactttcgatggtagtcgctgtgcctaccatggtgaccacgggtgacggggaatcagggttcgattccggagagggagcctgagaaacggctaccacatccaaggaaggcagcaggcgcgcaaattacccactcccgacccggggaggtagtgacgaaaaataacaatacaggactctttgaggcc

SEQ ID NO: 3 - DNA, 176 bp, Bos taurus
gctatcctgagggaaacttcggagggaaccagctactagatggttcgattagtctttcgcccctatacccaggtcggacgaccgatttgcacgtcaggaccgctacggacctccaccagagtttcctctggcttcgccctgcccaggcatagttcaccatctttcgggtcctaaca

SEQ ID NO: 4 - DNA, 225 bp, Bos taurus
ggtctgtgcgtttgcgagagcgccagctatcctgagggaaacttcggagggaaccagctactagatggttcgattagtctttcgcccctatacccaggtcggacgaccgatttgcacgtcaggaccgctacggacctccaccagagtttcctctggcttcgccctgcccaggcatagttcaccatctttcgggtcctaacacgtgcgctcatgctccacctcccc

SEQ ID NO: 5 - DNA, 188 bp, Bos taurus
ttcgctggatagtaggtagggacagtgggaatctcgttcatccattcatgcgcgtcactaattagatgacgaggcatttggctaccttaagagagtcatagttactcccgccgtttacccgcgcttcattgaatttcttcactttgacattcagagcactgggcagaaatcacatcgcgtcaacaccc

SEQ ID NO: 6 - DNA, 237 bp, Bos taurus
ccaagcccgttcccttggctgtggtttcgctggatagtaggtagggacagtgggaatctcgttcatccattcatgcgcgtcactaattagatgacgaggcatttggctaccttaagagagtcatagttactcccgccgtttacccgcgcttcattgaatttcttcactttgacattcagagcactgggcagaaatcacatcgcgtcaacacccgccgcgggccttcgcgatgctttg

SEQ ID NO: 7 - DNA, 166 bp, Bos taurus
agatggatttagagtgtgtggctgttgtgtgtggttcagaattctgagtagtcctttcagggatcacccaaaagctcagacaaagcgtgagcccttcacgtggactgtgctgtgcgtcagaaagataatacgagccacctacacaatctaaaatttcctaaaaacc

SEQ ID NO: 8 - DNA, 215 bp, Bos taurus
tgaacactgaatgtgtttgcattatagatggatttagagtgtgtggctgttgtgtgtggttcagaattctgagtagtcctttcagggatcacccaaaagctcagacaaagcgtgagcccttcacgtggactgtgctgtgcgtcagaaagataatacgagccacctacacaatctaaaatttcctaaaaaccaacgggctactcgacgtcctcctt

SEQ ID NO: 9 - DNA, 164 bp, Bos taurus
agctgaagtggtgggatagtgtagtttgtgtgtcataggaatctcctcaaaggatgatgtcatcagtgtgaggtcctaattgtgtcctagaaaaagttagagctggtgaaaatgttgccttcaaagactgtgtgagatggcaggtgtggacactggccccactg

SEQ ID NO: 10 - DNA, 213 bp, Bos taurus
tttaccaaaaagttttcaggagcttagctgaagtggtgggatagtgtagtttgtgtgtcataggaatctcctcaaaggatgatgtcatcagtgtgaggtcctaattgtgtcctagaaaaagttagagctggtgaaaatgttgccttcaaagactgtgtgagatggcaggtgtggacactggccccactgagtagacaggtgacggtgtatcgt

SEQ ID NO: 11 - DNA, 150 bp, Bos taurus
gggtgttggttctccttgcaggtcctggagatacagccaaaggcccagtttttgagagaactcttcctaactgtcttcttgtctgaaactaatgtagaagagagattttcactagtagtagtttagcttatttagcctgatttccaccac

SEQ ID NO: 12 - DNA, 199 bp, Bos taurus
ttttggctttgtgggtggtagacatgggtgttggttctccttgcaggtcctggagatacagccaaaggcccagtttttgagagaactcttcctaactgtcttcttgtctgaaactaatgtagaagagagattttcactagtagtagtttagcttatttagcctgatttccaccacttacattcagcagcatgttcatgg

SEQ ID NO: 13 - DNA, 189 bp, Bos taurus
gttgtttgtaatattacttaggtatttttaacatttttattggaaagtgaaagttagttttggttgtgctgggttttcattgctgtgtgcagtctttccctagttgcagagtgtgggggctactctttgttttaggcttttcattgtggtggcttctcttgttgcagagcctgggctgcagagtgtgtg

SEQ ID NO: 14 - DNA, 238 bp, Bos taurus
aaattgtctggtttatatgcatagagttgtttgtaatattacttaggtatttttaacatttttattggaaagtgaaagttagttttggttgtgctgggttttcattgctgtgtgcagtctttccctagttgcagagtgtgggggctactctttgttttaggcttttcattgtggtggcttctcttgttgcagagcctgggctgcagagtgtgtgggcttcagtagttgtggcacatgg

SEQ ID NO: 15 - DNA, 131 bp, Bos taurus
gatcttattctttgaagtaatgttatgaatcttatcggacaaagccagacttcttcatttgggagtagatttggaagagaacacttggtctctcagcactccccttctcaggaaggttacagaaaccaaaa

SEQ ID NO: 16 - DNA, 180 bp, Bos taurus
ttctgactctgcagtacctaaatcagatcttattctttgaagtaatgttatgaatcttatcggacaaagccagacttcttcatttgggagtagatttggaagagaacacttggtctctcagcactccccttctcaggaaggttacagaaaccaaaataagacctaccagaattggggtgg

SEQ ID NO: 17 - DNA, 336 bp, Bos taurus
gtttgtaacctgggacccttgagttaattctttttcttgttatagcccaccacacctttgctctgtaggaatgcaactttatctaatgcttttttggagggtggctcctgaccaaccacctttagagaaaaataagttttctgaagaaaaggtcttaaaatgttaacaggcctccgggccagaagatgatgcaaatcacctaagcttttgcatatgataagtttgcaggaagaaagcctggtttgctgcaagactcgaccccttcccccattatcctctatgcataacttaaggtataaaaactactttgaaaaataaagtgcgggccttgttcac

SEQ ID NO: 18 - DNA, 385 bp, Bos taurus
attggtgtaccgtagtagctttaatgtttgtaacctgggacccttgagttaattctttttcttgttatagcccaccacacctttgctctgtaggaatgcaactttatctaatgcttttttggagggtggctcctgaccaaccacctttagagaaaaataagttttctgaagaaaaggtcttaaaatgttaacaggcctccgggccagaagatgatgcaaatcacctaagcttttgcatatgataagtttgcaggaagaaagcctggtttgctgcaagactcgaccccttcccccattatcctctatgcataacttaaggtataaaaactactttgaaaaataaagtgcgggccttgttcaccgaaacttggtctcaccatgtcgt

SEQ ID NO: 19 - DNA, 323 bp, Bos taurus
gtgtccctggcagtgtattgattaatataattggtgtaagtagtagctttaatgtttgtaacctgggacccttgagttaattctttttcttgttatagcccaccacacctttgctctgtaggaatgcaactttatctaatgcttttttggagggtggctcctgaccaaccacctttagagaaaaataagttttctgaagaaaaggtcttaaaatgttaacaggcctccgggccagaagatgatgcaaatcacctaagcttttgcatatgataagtttgcaggaagaaagcctggtttgctgcaagactcgaccccttccccca

SEQ ID NO: 20 - DNA, 372 bp, Bos taurus
ctaagtttccatatccattagtgctgtgtccctggcagtgtattgattaatataattggtgtaagtagtagctttaatgtttgtaacctgggacccttgagttaattctttttcttgttatagcccaccacacctttgctctgtaggaatgcaactttatctaatgcttttttggagggtggctcctgaccaaccacctttagagaaaaataagttttctgaagaaaaggtcttaaaatgttaacaggcctccgggccagaagatgatgcaaatcacctaagcttttgcatatgataagtttgcaggaagaaagcctggtttgctgcaagactcgaccccttcccccattatccttctatgcataacttaag

SEQ ID NO: 21 - DNA, 257 bp, Bos taurus
actacttacaccaattatattaatcaatacactgccagggacacagcaggtaagggatatggaaacttagcagcaaacaatggcccaacaagtgaaaaaccattcaccaatacaatttctaatcaatcttgtaactactcaaaagaatctgtgtttagacagtttagaacatctcctgcctctcacagttgggaggctctgaacaatcccatgtggccggaaaaacctattcaggcaggctagaggatttccaaagg

SEQ ID NO: 22 - DNA, 306 bp, Bos taurus
gtcccaggttacaaacattaaagctactacttacaccaattatattaatcaatacactgccagggacacagcaggtaagggatatggaaacttagcagcaaacaatggcccaacaagtgaaaaaccattcaccaatacaatttctaatcaatcttgtaactactcaaaagaatctgtgtttagacagtttagaacatctcctgcctctcacagttgggaggctctgaacaatcccatgtggccggaaaaacctattcaggcaggctagaggatttccaaaggagtttgtaggttaaacactgtcac

SEQ ID NO: 23 - DNA, 404 bp, Bos taurus
catcatcttctggcccggaggcctgttaacattttaagaccttttcttcagaaaacttatttttctctaaaggtggttggtcaggagccaccctccaaaaaagcattagataaagttgcattcctacagagcaaaggtgtggtgggctataacaagaaaaagaattaactcaagggtcccaggttacaaacattaaagctactacttacaccaattatattaatcaatacactgccagggacacagcaggtaagggatatggaaacttagcagcaaacaatggcccaacaagtgaaaaaccattcaccaatacaatttctaatcaatcttgtaactcctcaaaagaatctgtgtttagacagtttagaacatctcctgcctctcacagttgggaggctctgaac

SEQ ID NO: 24 - DNA, 453 bp, Bos taurus
  modified_base (1)..(7): a, c, t, g, unknown or other
nnnnnnnaaaagcttaggtgatttgcatcatcttctggcccggaggcctgttaacattttaagaccttttcttcagaaaacttatttttctctaaaggtggttggtcaggagccaccctccaaaaaagcattagataaagttgcattcctacagagcaaaggtgtggtgggctataacaagaaaaagaattaactcaagggtcccaggttacaaacattaaagctactacttacaccaattatattaatcaatacactgccagggacacagcaggtaagggatatggaaacttagcagcaaacaatggcccaacaagtgaaaaaccattcaccaatacaatttctaatcaatcttgtaactcctcaaaagaatctgtgtttagacagtttagaacatctcctgcctctcacagttgggaggctctgaacaatcacatgtggccggaaaaacct

SEQ ID NO: 25 - DNA, 319 bp, Bos taurus
accaagtttcggtgaacaaggcccgcactttatttttcaaagtagtttttataccttaagttatgcatagaggataatgggggaaggggtcgagtcttgcagcaaaccaggctttcttcctgcaaacttatcatatgcaaaagcttaggtgatttgcatcatcttctggcccggaggcctgttaacattttaagaccttttcttcagaaaacttatttttctctaaaggtggttggtcaggagccaccctccaaaaaagcattagataaagttgcattcctacagagcaaaggtgtggtgggctataacaagaaaaaga

SEQ ID NO: 26 - DNA, 368 bp, Bos taurus
  modified_base (345)..(368): a, c, t, g, unknown or other
gtaagagaaagaacgacatggtgagaccaagtttcggtgaacaaggcccgcactttatttttcaaagtagtttttataccttaagttatgcatagaggataatgggggaaggggtcgagtcttgcagcaaaccaggctttcttcctgcaaacttatcatatgcaaaagcttaggtgatttgcatcatcttctggcccggaggcctgttaacattttaagaccttttcttcagaaaacttatttttctctaaaggtggttggtcaggagccaccctccaaaaaagcattagataaagttgcattcctacagagcaaaggtgtggtgggctataacaagaaaaagannnnnnnnnnnnnnnnnnnnnnnn

SEQ ID NO: 27 - DNA, 297 bp, Bos taurus
acacctttgctctgtaggaatgcaactttatctaatgcttttttggagggtggctcctgaccaaccacctttagagaaaaataagttttctgaagaaaaggtcttaaaatgttaacaggcctccgggccagaagatgatgcaaatcacctaagcttttgcatatgataagtttgcaggaagaaagcctggtttgctgcaagactcgaccccttcccccattatcctctatgcataacttaaggtataaaaactactttgaaaaataaagtgcgggccttgttcaccgaaacttggtc

SEQ ID NO: 28 - DNA, 346 bp, Bos taurus
  modified_base (1)..(25): a, c, t, g, unknown or other
nnnnnnnnnnnnnnnnnnnnnnnnnacacctttgctctgtaggaatgcaactttatctaatgcttttttggagggtggctcctgaccaaccacctttagagaaaaataagttttctgaagaaaaggtcttaaaatgttaacaggcctccgggccagaagatgatgcaaatcacctaagcttttgcatatgataagtttgcaggaagaaagcctggtttgctgcaagactcgaccccttcccccattatcctctatgcataacttaaggtataaaaactactttgaaaaataaagtgcgggccttgttcaccgaaacttggtctcaccatgtcgttctttctcttac

SEQ ID NO: 29 - DNA, 201 bp, Bos taurus
tgcatagaggataatgggggaaggggtcgagtcttgcagcaaaccaggctttcttcctgcaaacttatcatatgcaaaagcttaggtgatttgcatcatcttctggcccggaggcctgttaacattttaagaccttttcttcagaaaacttatttttctctaaaggtggttggtcaggagccaccctccaaaaaagcatta

SEQ ID NO: 30 - DNA, 250 bp, Bos taurus
aaagtagtttttataccttaaattatgcatagaggataatgggggaaggggtcgagtcttgcagcaaaccaggctttcttcctgcaaacttatcatatgcaaaagcttaggtgatttgcatcatcttctggcccggaggcctgttaacattttaagaccttttcttcagaaaacttatttttctctaaaggtggttggtcaggagccaccctccaaaaaagcattagccagagttgcattcctacagagc

SEQ ID NO: 31 - DNA, 450 bp, Bos taurus
gaaatcctctagcctgcctgaataggtttttccggccacatgggattgttcagagcctcccaactgtgagaggcaggagatgttctaaactgtctaaacacagattcttttgagtagttacaagattgattagaaattgtattggtgaatggtttttcacttcttgggccattgtttgctgctaagtttccatatcccttacctgctgtgtccctggcagtgtattgattaatataattggtgtaagtagtagctttaatgtttgtaacctgggacccttgagttaattctttttcttgttatagcccaccacacctttgctctgtaggaatgcaactttatctaatgcttttttggagggtggctcctgaccaaccacctttagagaaaaataagttttctgaagaaaaggtcttaaaatgttaacaggcctccgggccagaagatgat

SEQ ID NO: 32 - DNA, 498 bp, Bos taurus
  modified_base (482)..(498): a, c, t, g, unknown or other
agtgtttaacctacaaactcctttggaaatcctctagcctgcctgaataggtttttccggccacatgggattgttcagagcctcccaactgtgagaggcaggagatgttctaaactgtctaaacacagattcttttgagtagttacaagattgattagaaattgtattggtgaatggtttttcacttcttgggccattgtttgctgctaagtttccatatcccttacctgctgtgtccctggcagtgtattgattaatataattggtgtaagtagtagctttaatgtttgtaacctgggacccttgagttaattctttttcttgttatagcccaccacacctttgctctgtaggaatgcaactttatctaatgcttttttggagggtggctcctgaccaaccacctttagagaaaaataagttttctgaagaaaaggtcttaaaatgttaacaggcctccgggccagaagatgatgcaaatnnnnnnnnnnnnnnnnn

SEQ ID NO: 33 - DNA, 200 bp, Bos taurus
accttagaacaaaccgaggcactatgaacattttgtgcttcatgttgatgactcttagacatgtctacagtagaggagcaaaaacaaaactactagatatttcatattgactagttcccagttcacgggactctgacattccctgaggtcaaagttttcttgtattggaagcagttgggtttgcaagggctgccttgtct

SEQ ID NO: 34 - DNA, 249 bp, Bos taurus
cagatagaacgatatagcaatttttaccttagaacaaaccgaggcactatgaacattttgtgcttcatgttgatgactcttagacatgtctacagtagaggagcaaaaacaaaactactagatatttcatattgactagttcccagttcacgggactctgacattccctgaggtcaaagttttcttgtattggaagcagttgggtttgcaagggctgccttgtcttgagaccattgaaataagaactca

SEQ ID NO: 35 - DNA, 200 bp, Bos taurus
agaaaactttgacctcagggaatgtcagagtcccgtgaactgggaactagtcaatatgaaatatctagtagttttgtttttgctcctctactgtagacatgtctaagagtcatcaacatgaagcacaaaatgttcatagtgcctcggtttgttctaaggtaaaaattgctattgctatatcgttctatctgtcacaagct

SEQ ID NO: 36 - DNA, 249 bp, Bos taurus
tgcaaacccaactgcttccaatacaagaaaactttgacctcagggaatgtcagagtcccgtgaactgggaactagtcaatatgaaatatctagtagttttgtttttgctcctctactgtagacatgtctaagagtcatcaacatgaagcacaaaatgttcatagtgcctcggtttgttctaaggtaaaaattgctattgctatatcgttctatctgtcacaagcttgtcaacaagaaagtacctcgaaa

SEQ ID NO: 37 - DNA, 200 bp, Bos taurus
tggaccccagaaaagagtgttgtgcctagcacaaggtttcttgagaggttgaactgcctttgctaatggattttaagtttctttacctcttccgtgatctcttctaggttgacctagaagagaaatcttttgttagctttggctaagtggagaagtattgcttcacggtgacttgtgtgatcctacctgactgttctaaa

SEQ ID NO: 38 - DNA, 249 bp, Bos taurus
tgggatcaggaggttgtagggaacgtggaccccagaaaagagtgttgtgcctagcacaaggtttcttgagaggttgaactgcctttgctaatggattttaagtttctttacctcttccgtgatctcttctaggttgacctagaagagaaatcttttgttagctttggctaagtggagaagtattgcttcacggtgacttgtgtgatcctacctgactgttctaaacccttttgatatctatgcactgct

SEQ ID NO: 39 - DNA, 200 bp, Bos taurus
cagaaaagaatgttgtgcctagcacaaggtttcttgagaggttgaactgcctttgctaatggattttaagtttctttacctcttccgtgatctcttctaggttgacctagaacagaaatcttttgttagctttggctaagtggagaagtattgcttcacggtgacttgtgtgatcctacctgactgttctaaaccctttt

SEQ ID NO: 40 - DNA, 249 bp, Bos taurus
ggaggttgtagggatagtggacccccagaaaagaatgttgtgcctagcacaaggtttcttgagaggttgaactgcctttgctaatggattttaagtttctttacctcttccgtgatctcttctaggttgacctagaacagaaatcttttgttagctttggctaagtggagaagtattgcttcacggtgacttgtgtgatcctacctgactgttctaaacccttttgatatctatgcactgttgctgctg

SEQ ID NO: 41 - DNA, 200 bp, Bos taurus
gtatcctggcacatacatgtactgcctcacccctaacctcagtcatgtgttccaagaacttctgtccgtttatttatttgtttttggtaacattgaattcgttttgactatgctgggtctttgctgctcgctggaagttgctaggacctctggactgcagtctcccatctcttgctgacctcttgttgacctctgcagaa

SEQ ID NO: 42 - DNA, 249 bp, Bos taurus
tctacgttaagagtatactttaagagtatcctggcacatacatgtactgcctcacccctaacctcagtcatgtgttccaagaacttctgtccgtttatttatttgtttttggtaacattgaattcgttttgactatgctgggtctttgctgctcgctggaagttgctaggacctctggactgcagtctcccatctcttgctgacctcttgttgacctctgcagaatcctggttggtgggagcttgttag

SEQ ID NO: 43 - DNA, 200 bp, Bos taurus
acaagaaaactttgacctcagggaatgtcagagtcccgtgaactgggaactagtcaatatgaaatatctagtagttttgtttttgctcctctactgtagacatgtctaagagtcatcaacacgaagcacaaaatgttcatagtgcctcggtttgttctgaggtaaaaattgctatatcgttctatctgtcacaagcttgt

SEQ ID NO: 44 - DNA, 249 bp, Bos taurus
ccttgcaaacccaactgcttccaatacaagaaaactttgacctcagggaatgtcagagtcccgtgaactgggaactagtcaatatgaaatatctagtagttttgtttttgctcctctactgtagacatgtctaagagtcatcaacacgaagcacaaaatgttcatagtgcctcggtttgttctgaggtaaaaattgctatatcgttctatctgtcacaagcttgtcaacaagaaagtacctcgaaagaa

SEQ ID NO: 45 - DNA, 200 bp, Bos taurus
agcctgcctaaatcattgacttctagccacctttgggatgccaatgaccacccagccttaccaggccacagaggagagaaaacatttaccacatgaggtccgggtaaggaacaaggaactaacgagctcccaccaaccaggattctgcagaggtcaacaagaggtcagcaagagatgggagactgcagtccagaggtcct

SEQ ID NO: 46 - DNA, 249 bp, Bos taurus
ttgccttctcccatatctattcacaagcctgcctaaatcattgacttctagccacctttgggatgccaatgaccacccagccttaccaggccacagaggagagaaaacatttaccacatgaggtccgggtaaggaacaaggaactaacgagctcccaccaaccaggattctgcagaggtcaacaagaggtcagcaagagatgggagactgcagtccagaggtcctagcaaattccagcgagcagctaag

SEQ ID NO: 47 - DNA, 200 bp, Bos taurus
tgactcttagacatgtctacagtagaggagcaaaaacaaaactactagatatttcatattgactagttcccagttcacgggactctgacattccctgaggtcaaagttttcttgtattggaagcagttgggtttgcaagggctgccttgtcttgagaccattgaactaagaactcagaacttgagcactattatcaaaaa

SEQ ID NO: 48 - DNA, 249 bp, Bos taurus
atgaacattttgtgcttcatgttgatgactcttagacatgtctacagtagaggagcaaaaacaaaactactagatatttcatattgactagttcccagttcacgggactctgacattccctgaggtcaaagttttcttgtattggaagcagttgggtttgcaagggctgccttgtcttgagaccattgaactaagaactcagaacttgagcactattatcaaaaatcacaaggctcacactgacacaga

SEQ ID NO: 49 - DNA, 200 bp, Bos taurus
ttgggtgtcacagtggaggtgtagaagaggtctgagagtgcaaggcagttaaggaagaagtacatgggctgggtgattagctgactgcatgtaatagaaatcacaatgatcaaattccccaaccaaatagccaggtaacagaataagaaaaataaaaagcagaggatctggactttcttgtccatagaaagttctaagag

SEQ ID NO: 50 - DNA, 249 bp, Bos taurus
ctccatcagtaagtcagtcatgagtttgggtgtcacagtggaggtgtagaagaggtctgagagtgcaaggcagttaaggaagaagtacatgggctgggtgattagctgactgcatgtaatagaaatcacaatgatcaaattccccaaccaaatagccaggtaacagaataagaaaaataaaaagcagaggatctggactttcttgtccatagaaagttctaagaggataaattcagtaacattatttct

SEQ ID NO: 51 - DNA, 200 bp, Bos taurus
cacccctaacctcagtcgtgtgttccaagaacttctgtccgtttatttatttgttttgggtaacattgaattcgttttgactatgctgggtctttgctgctcgctggaagttgctaggacctctggactgcagtctcccatctcttgctgacctcttgttgacctctgcagaatcctggttgctgggagcttgttagttc

SEQ ID NO: 52 - DNA, 249 bp, Bos taurus
atcctggcacatacatgtactgcctcacccctaacctcagtcgtgtgttccaagaacttctgtccgtttatttatttgttttgggtaacattgaattcgttttgactatgctgggtctttgctgctcgctggaagttgctaggacctctggactgcagtctcccatctcttgctgacctcttgttgacctctgcagaatcctggttgctgggagcttgttagttccttgttccttacccggaccacctg

SEQ ID NO: 53 - DNA, 200 bp, Bos taurus
caagaggtcagcaagagatgggagactgcagtccagaggtcctagcaacttccagcgagcagcaaagacccagcatagtcaaaacgaattcaatgttaccaaaaacaaataaataaacggacagaagttcttggaacacatgactgaggttaggggtgaggcagtacatgtatgtgccaggatactcttaaagtatactc

SEQ ID NO: 54 - DNA, 249 bp, Bos taurus
ccaaccaggattctgcagaggtcaacaagaggtcagcaagagatgggagactgcagtccagaggtcctagcaacttccagcgagcagcaaagacccagcatagtcaaaacgaattcaatgttaccaaaaacaaataaataaacggacagaagttcttggaacacatgactgaggttaggggtgaggcagtacatgtatgtgccaggatactcttaaagtatactcttaacgtagatgaggagaacaagg

SEQ ID NO: 55 - DNA, 200 bp, Bos taurus
gacaaggcagcccttgcaaacccaactgcttccaatacaagaaaactttgacctcagggaatgtcagagtcccgtgaactgggaactagtcaatatgaaatatctggtagttttgtttttgctcctctactgtagacatgtctaagagtcatcaacatgaagcacaaaatgttcatagtgcctcggtttgttctaaggta

SEQ ID NO: 56 - DNA, 249 bp, Bos taurus
tgagttcttatttcaatggtctcaagacaaggcagcccttgcaaacccaactgcttccaatacaagaaaactttgacctcagggaatgtcagagtcccgtgaactgggaactagtcaatatgaaatatctggtagttttgtttttgctcctctactgtagacatgtctaagagtcatcaacatgaagcacaaaatgttcatagtgcctcggtttgttctaaggtaaaaattgctatatcgttctatctg

SEQ ID NO: 57 - DNA, 200 bp, Bos taurus
tcgctggaggttgctaggacctctggactgcagtctcccatctcttgctgacctcttgttgacctctgcagaatcctggttgctgggagcttgttagttccttgttccttacccggaccacctgtggttaagtgttttctctcctctgtggcctggtaaggctgggtggtcattggcatcccaaaggtggctagaggtca

SEQ ID NO: 58 - DNA, 200 bp, Bos taurus
catatctattcacaagcctgcctaaatcattgacctctagccacctttgggatgccaatgaccacccagccttaccaggccacagaggagagaaaacacttaaccacaggtggtccgggtaaggaacaaggaactaacaagctcccagcaaccaggattctgcagaggtcaacaagaggtcagcaagagatgggagactg

SEQ ID NO: 59 - DNA, 201 bp, Bos taurus
gaaacaggaagcccagaggtgcccctgtagtttggcgaaggccttaggaggtcacttggaaatgccgctgtctgtgagggggggagggggcacatgtgcacagagctgtgcaggcttgtcctttgcatggatgttctttgtagaaatccacctcggagccatctcctgatgtgtaagaatttcccagacaaaccttgagct

SEQ ID NO: 60 - DNA, 200 bp, Bos taurus
gggtgtgtgtgtgtgggggggggagcccttctttccattccacatgagacccagtggacaatccaggccctgcccactgcattgagtgtgcagcaacaatatcaacggtaatgtgggtgaacaacatgtattcctggaatccactccgttggctactgactgtgtgtggtggtggtctggaacctagacctggaagcttt

SEQ ID NO: 61 - DNA, 200 bp, Bos taurus
gtttatctaaaagctcatatcacatttaatttttcgttttgttcctgttctttcgaggtactttcttgttgacaagcttgtgacagatagaacgatatagcaatttttaccttagaacaaaccgaggcactatgaacattttgtgcttcatgttgatgactcttagacatgtctacagtagaggagcaaaaacaaaacta

SEQ ID NO: 62 - DNA, 301 bp, Bos taurus
gatactcgccagctgagccgcgcgacacctgcatgctcatctgcctgatactcgccagctgagccgcgcgacatctgcccgctcatcagcctgatactcgccagctgagccccgcgacacctgcccgctcatcagcctgatactcgccagctgagccccgcgacacctgcccgctcatctgcctgatactcgccagctgagccgcgcgacacctgcccgctcatctgcctgatactcgccagctgagccgcgcgacacctgcccgctcatctgcctgatactctccaggtgaatcgcgcca

SEQ ID NO: 63 - DNA, 201 bp, Bos taurus
cgaccggaaggtcgggaaccccttgcagacaaagcaggggagtcgaccctcccgtccagatcaggaggggagaaagggctcagaggagggggtgccggaaaacctcagggttcctctcgagggagaccgggatttcggggaactttgtgggtcgcatcaagggtgccaagtgccctttcgacctccaattgtggcttcttg

SEQ ID NO: 64 - DNA, 20 bp, Artificial Sequence
  Description of Artificial Sequence Synthetic primer
ggaatcagggttcgattccg

SEQ ID NO: 65 - DNA, 20 bp, Artificial Sequence
  Description of Artificial Sequence Synthetic primer
ggtcgggagtgggtaatttg

SEQ ID NO: 66 - DNA, 18 bp, Artificial Sequence
  Description of Artificial Sequence Synthetic primer
ggtcggacgaccgatttg

SEQ ID NO: 67 - DNA, 22 bp, Artificial Sequence
  Description of Artificial Sequence Synthetic primer
acccgaaagatggtgaactatg

SEQ ID NO: 68 - DNA, 22 bp, Artificial Sequence
  Description of Artificial Sequence Synthetic primer
gacagtgggaatctcgttcatc

SEQ ID NO: 69 - DNA, 22 bp, Artificial Sequence
  Description of Artificial Sequence Synthetic primer
gtaaacggcgggagtaactatg

SEQ ID NO: 70 - DNA, 22 bp, Artificial Sequence
  Description of Artificial Sequence Synthetic primer
tctgagtagtcctttcagggat

SEQ ID NO: 71 - DNA, 24 bp, Artificial Sequence
  Description of Artificial Sequence Synthetic primer
gtaggtggctcgtattatctttct

SEQ ID NO: 72 - DNA, 22 bp, Artificial Sequence
  Description of Artificial Sequence Synthetic primer
gtggtgggatagtgtagtttgt

SEQ ID NO: 73 - DNA, 22 bp, Artificial Sequence
  Description of Artificial Sequence Synthetic primer
cacagtctttgaaggcaacatt

SEQ ID NO: 74 - DNA, 19 bp, Artificial Sequence
  Description of Artificial Sequence Synthetic primer
cttgcaggtcctggagata

SEQ ID NO: 75 - DNA, 25 bp, Artificial Sequence
  Description of Artificial Sequence Synthetic primer
ctctcttctacattagtttcagaca

SEQ ID NO: 76 - DNA, 19 bp, Artificial Sequence
  Description of Artificial Sequence Synthetic primer
tcattgctgtgtgcagtct

SEQ ID NO: 77 - DNA, 20 bp, Artificial Sequence
  Description of Artificial Sequence Synthetic primer
aggctctgcaacaagagaag

SEQ ID NO: 78 - DNA, 21 bp, Artificial Sequence
  Description of Artificial Sequence Synthetic primer
tcttatcggacaaagccagac

SEQ ID NO: 79 - DNA, 22 bp, Artificial Sequence
  Description of Artificial Sequence Synthetic primer
tggtttctgtaaccttcctgag

SEQ ID NO: 80 - DNA, 21 bp, Artificial Sequence
  Description of Artificial Sequence Synthetic primer
ctcctgaccaaccacctttag

SEQ ID NO: 81 - DNA, 23 bp, Artificial Sequence
  Description of Artificial Sequence Synthetic primer
ggctttcttcctgcaaacttatc

SEQ ID NO: 82 - DNA, 21 bp, Artificial Sequence
  Description of Artificial Sequence Synthetic primer
gtccctggcagtgtattgatt

SEQ ID NO: 83 - DNA, 21 bp, Artificial Sequence
  Description of Artificial Sequence Synthetic primer
caaaggtgtggtgggctataa

SEQ ID NO: 84 - DNA, 23 bp, Artificial Sequence
  Description of Artificial Sequence Synthetic primer
gtttagaacatctcctgcctctc

SEQ ID NO: 85 - DNA, 21 bp, Artificial Sequence
  Description of Artificial Sequence Synthetic primer
atcctctagcctgcctgaata

SEQ ID NO: 86 - DNA, 21 bp, Artificial Sequence
  Description of Artificial Sequence Synthetic primer
caaaggtgtggtgggctataa

SEQ ID NO: 87 - DNA, 21 bp, Artificial Sequence
  Description of Artificial Sequence Synthetic primer
gtccctggcagtgtattgatt

SEQ ID NO: 88 - DNA, 24 bp, Artificial Sequence
  Description of Artificial Sequence Synthetic primer
gcttgttagttccttgttccttac

SEQ ID NO: 89 - DNA, 20 bp, Artificial Sequence
  Description of Artificial Sequence Synthetic primer
ctctagccacctttgggatg

SEQ ID NO: 90 - DNA, 22 bp, Artificial Sequence
  Description of Artificial Sequence Synthetic primer
cgggtaaggaacaaggaactaa

SEQ ID NO: 91 - DNA, 21 bp, Artificial Sequence
  Description of Artificial Sequence Synthetic primer
catctcttgctgacctcttgt

SEQ ID NO: 92 - DNA, 22 bp, Artificial Sequence
  Description of Artificial Sequence Synthetic primer
ccttaggaggtcacttggaaat

SEQ ID NO: 93 - DNA, 22 bp, Artificial Sequence
  Description of Artificial Sequence Synthetic primer
gaacatccatgcaaaggacaag

SEQ ID NO: 94 - DNA, 20 bp, Artificial Sequence
  Description of Artificial Sequence Synthetic primer
gagcccttctttccattcca

SEQ ID NO: 95 - DNA, 22 bp, Artificial Sequence
  Description of Artificial Sequence Synthetic primer
ttcacccacattaccgttgata

SEQ ID NO: 96 - DNA, 23 bp, Artificial Sequence
  Description of Artificial Sequence Synthetic primer
cctgttctttcgaggtactttct

SEQ ID NO: 97 - DNA, 22 bp, Artificial Sequence
  Description of Artificial Sequence Synthetic primer
catagtgcctcggtttgttcta

SEQ ID NO: 98 - DNA, 17 bp, Artificial Sequence
  Description of Artificial Sequence Synthetic primer
acatctgcccgctcatc

SEQ ID NO: 99 - DNA, 19 bp, Artificial Sequence
  Description of Artificial Sequence Synthetic primer
ctcagctggcgagtatcag

SEQ ID NO: 100 - DNA, 19 bp, Artificial Sequence
  Description of Artificial Sequence Synthetic primer
aaacctcagggttcctctc

SEQ ID NO: 101 - DNA, 20 bp, Artificial Sequence
  Description of Artificial Sequence Synthetic primer
ccacaattggaggtcgaaag

SEQ ID NO: 102 - DNA, 18 bp, Artificial Sequence
  Description of Artificial Sequence Synthetic primer
aacaaggcccgcacttta

SEQ ID NO: 103 - DNA, 25 bp, Artificial Sequence
  Description of Artificial Sequence Synthetic primer
gcatatgataagtttgcaggaagaa

SEQ ID NO: 104 - DNA, 22 bp, Artificial Sequence
  Description of Artificial Sequence Synthetic primer
gccagaagatgatgcaaatcac

SEQ ID NO: 105 - DNA, 27 bp, Artificial Sequence
  Description of Artificial Sequence Synthetic primer
cttaagttatgcatagaggataatggg

SEQ ID NO: 106 - DNA, 24 bp, Artificial Sequence
  Description of Artificial Sequence Synthetic primer
gcttaggtgatttgcatcatcttc

SEQ ID NO: 107 - DNA, 17 bp, Artificial Sequence
  Description of Artificial Sequence Synthetic primer
tttggagggtggctcct

SEQ ID NO: 108 - DNA, 21 bp, Artificial Sequence
  Description of Artificial Sequence Synthetic primer
gtccctggcagtgtattgatt

SEQ ID NO: 109 - DNA, 21 bp, Artificial Sequence
  Description of Artificial Sequence Synthetic primer
caaaggtgtggtgggctataa

SEQ ID NO: 110 - DNA, 22 bp, Artificial Sequence
  Description of Artificial Sequence Synthetic primer
attgactagttcccagttcacg

SEQ ID NO: 111 - DNA, 17 bp, Artificial Sequence
  Description of Artificial Sequence Synthetic primer
agcccttgcaaacccaa

SEQ ID NO: 112 - DNA, 22 bp, Artificial Sequence
  Description of Artificial Sequence Synthetic primer
gagtcatcaacatgaagcacaa

SEQ ID NO: 113 - DNA, 23 bp, Artificial Sequence
  Description of Artificial Sequence Synthetic primer
agcttgtgacagatagaacgata

SEQ ID NO: 114 - DNA, 23 bp, Artificial Sequence
  Description of Artificial Sequence Synthetic primer
cttccgtgatctcttctaggttg

SEQ ID NO: 115 - DNA, 22 bp, Artificial Sequence
  Description of Artificial Sequence Synthetic primer
gtcaggtaggatcacacaagtc

SEQ ID NO: 116 - DNA, 24 bp, Artificial Sequence
  Description of Artificial Sequence Synthetic primer
aggttgacctagaacagaaatctt

SEQ ID NO: 117 - DNA, 22 bp, Artificial Sequence
  Description of Artificial Sequence Synthetic primer
gggtttagaacagtcaggtagg

SEQ ID NO: 118 - DNA, 20 bp, Artificial Sequence
  Description of Artificial Sequence Synthetic primer
gactatgctgggtctttgct

SEQ ID NO: 119 - DNA, 21 bp, Artificial Sequence
  Description of Artificial Sequence Synthetic primer
ttctgcagaggtcaacaagag

SEQ ID NO: 120 - DNA, 21 bp, Artificial Sequence
  Description of Artificial Sequence Synthetic primer
actttgacctcagggaatgtc

SEQ ID NO: 121 - DNA, 22 bp, Artificial Sequence
  Description of Artificial Sequence Synthetic primer
tgcttcgtgttgatgactctta

SEQ ID NO: 122 - DNA, 21 bp, Artificial Sequence
  Description of Artificial Sequence Synthetic primer
ttgacttctagccacctttgg

SEQ ID NO: 123 - DNA, 21 bp, Artificial Sequence
  Description of Artificial Sequence Synthetic primer
gctcgttagttccttgttcct

SEQ ID NO: 124 - DNA, 22 bp, Artificial Sequence
  Description of Artificial Sequence Synthetic primer
attgactagttcccagttcacg

SEQ ID NO: 125 - DNA, 22 bp, Artificial Sequence
  Description of Artificial Sequence Synthetic primer
caaacccaactgcttccaatac

SEQ ID NO: 126 - DNA, 20 bp, Artificial Sequence
  Description of Artificial Sequence Synthetic primer
ctgagagtgcaaggcagtta

SEQ ID NO: 127 - DNA, 20 bp, Artificial Sequence
  Description of Artificial Sequence Synthetic primer
tacctggctatttggttggg

SEQ ID NO: 128 - DNA, 19 bp, Artificial Sequence
  Description of Artificial Sequence Synthetic primer
ctcgctggaagttgctagg

SEQ ID NO: 129 - DNA, 19 bp, Artificial Sequence
  Description of Artificial Sequence Synthetic primer
ctcccagcaaccaggattc

SEQ ID NO: 130 - DNA, 21 bp, Artificial Sequence
  Description of Artificial Sequence Synthetic primer
caagaggtcagcaagagatgg

SEQ ID NO: 131 - DNA, 23 bp, Artificial Sequence
  Description of Artificial Sequence Synthetic primer
tccaagaacttctgtccgtttat

SEQ ID NO: 132 - DNA, 17 bp, Artificial Sequence
  Description of Artificial Sequence Synthetic primer
agcccttgcaaacccaa

SEQ ID NO: 133 - DNA, 22 bp, Artificial Sequence
  Description of Artificial Sequence Synthetic primer
attgactagttcccagttcacg

SEQ ID NO: 134 - DNA, 240 bp, Bos taurus
gagtattcctctgtctcttcatcttgtttaaattggaatcacagataaatagcctggagacaaggattgagaagatgcaagaaaggtttaacaaggacctagaagaaataaaaaagagtcaatatataatgaataatgcaataaatgacattaaaaacactctggaggcaacaaatagtagaataacagaggcagaagataggattagtgaattagaagatagaatggtagaaataaatg

SEQ ID NO: 135 - DNA, 193 bp, Bos taurus
atggcacccagccttaccaggccacagaggagagaaaacacttaaccacaggtggtccgggtaaggaacaaggaactaacaagctcccagcaaccaggattctgcagaggtcaacaagaggtcagcaagagatgggagactgcagtccagaggtcctagcaacttccagcgagcagcaaagacccactaaccc

SEQ ID NO: 136 - DNA, 248 bp, Bos taurus
gtttgggttgtgttgttagttccttgttccttacccggaccacctgtggttaagtgttttctctcctctgtggcctggtaaggctgggtggtcattggcatcccaaaggtggctagaggtcaatgatttaggcaggcttgtgaatagatatgggagaaggcaatggcaccccactccagtactcttgcctggaaaatcccatggacccaagagcctggtaggctgcagtccatggggtcactaggagt

SEQ ID NO: 137 - DNA, 259 bp, Bos taurus
acatggagaagagaagagggaggagggagttagaaacttttcatctttaacgtagatgttttatcaatggtgctgtatagaaggagaagttttgaaactactgtaaaaataagaccgataaccggaagcaggagacttaagtccaaaccctgactccagggaactcctgactccaaggaacattaattgacgggagctcatcaaatgcatccataccgacactgaaacaaagcaccacacaagggccaataaattccag

SEQ ID NO: 138 - DNA, 292 bp, Bos taurus
gtctcctgtagacaacatatgtaggggtcttgtttttgtatccattcagccagtctttgtcttttggttggggcattcaacccatttacgtttaaggtaattactgataagtatgatcccgttgccatttactttattgtttggggttcgaatttatacaccatttttgtgtttcctgtctagagaatatcctttagtatttgttggagagctggtttggtggtgcagaattctctcagcttttgcttgtctgagaagcttttgatttctccttcatacttgaatgagatcc

SEQ ID NO: 139 - DNA, 236 bp, Bos taurus
gtataaccccgtgaacatctctgagtggtccagttgtggagtgcacataaggaagtgactactggctagcccactctctccactattgattcacctcatctcatttgggtcacctctaactccctcctccctcttctcttctccatgtaaccctgtgaacctctctgagtgaccctcactgtagagaaacttatcatctttaatgtagatgttttatcaatggtgctgtatagaag

SEQ ID NO: 140 - DNA, 265 bp, Bos taurus
attctatcttctaattcactaatcctatcttctgcctctgttattctactatttgttgcctccagagtgtttttaatttcatttattgcattattcattatatattgactctcttttatttcttctaggtccttgttaaacctttcttgcatcttctcgatcttagtctccaagctatttatctgtgattccattttgatttcaagattttggatcaatttcactatcattatagggaatctacctgataaagaattccaaataa

SEQ ID NO: 141 - DNA, 261 bp, Bos taurus
attttatctgatatgagtattgctactccggctctccaacaaatactaaaggatattctctagacaggaaacacaaaaagggtgtataaattcgaacccaaaacaataaagtaaatggcaacgggatcatacttatcaataattaccttaaacgtaaatgggttgaatgccccaaccaaaagacaaagactggctgaatggatacaaaaacaagacccctacatatgttgtctacaagagacccacttcagacctagggac

SEQ ID NO: 142 - DNA, 239 bp, Bos taurus
cttctatacagcaccattgataaaacatctacgttaaagatgaacagtttctctactgtgagggtcacccagagaggttcacagcgttacatggagaagagaagagggaggagggagttagaggtgacccgaatgagatgaggtggaatcaatagtggagtgagtggactagccaataatcacttccttatgtgcactccacaactggaccgctcatagatgttcacagagttatacag

SEQ ID NO: 143 - DNA, 247 bp, Bos taurus
gtttcacattgtcccagaggtctctgaggttgtcctaatttcttataattctttttccttgtttcctctctgcttcatttatttctaccattctatcttctacctcacttatcctatcttctgcctctgttattctactatttgttgcctccagagtgtttttaatttcatttattgcattattcattatatattgactcttttttatttcttctaggtccttgttaaacctttcttgcatcttctc

SEQ ID NO: 144 - DNA, 278 bp, Bos taurus
gtttgggtttgttgtttgattaatatgtgtcttggggtgtttccccttgggtttatcctgtttgggactctctggttttcttggacttgggtgattatttccttccccattttagggaagttttcaactattatctcctcaagtattttctcatggtctttctttttgtcttcttcttctgggacccctatgattcgaatgttgtagcgtttaatattgtcctggaggtctctgagattgtcctcatttcttttaattcgtttttcttttattttctc

SEQ ID NO: 145 - DNA, 212 bp, Bos taurus
ttcctgtggtcatgtatggatgtgagagttggactgtgaagaaggctgagcgccgaagaattgatgcttttgaactgtggtgttggagaagactctagagagtcccttggactgcaaggagatccaaccagtccatcctaaaggagatcagtcctgcgtgttgattggaaggaatgatgctaaagctgaaactccagtactttgaccacaca

SEQ ID NO: 146 - DNA, 202 bp, Bos taurus
tttcaaatgagtcagctcttcgcatcaggtggacaaaatattggagtttcagcttcaacatcaggactgatttcctttagaatggactggttggatctccttgcagtccaagggactctcaagagtcttctccaacaccacagttcaaaagcatcaattcttcagcactcagctttcttcacagtccaactctcacatccat

SEQ ID NO: 147 - DNA, 205 bp, Bos taurus
gatgtgagagttggactgtgaagaaggctgagcgccgaagaattgatgcttttgaactgtggtgttggagaagactcttgagagtcctttggactgcaaggagatccaaccagtccattctaaaggagatcagccctgggattcctttggagggaatgatgctaaagctgaaactccaatactttggccacctgatgcaaagaac

SEQ ID NO: 148 - DNA, 211 bp, Bos taurus
gtatagatgtgagagttggactgtgaagaaggctgagtgctgaagaattgatgcttttgaactgtggtgttggagaagactcttgagagtcccttggactgcaaggagatccaaccagtccattctaaaggaggtcagtcctgggtgttcattggaaggaatgatgctaaagctgaaactccagtactttggccacacaaaaaaccaaaca

SEQ ID NO: 149 - DNA, 238 bp, Bos taurus
gatagcatattaaaaatcagagacattactttgctgacaaaggttcatctagttaaggctatggtttttccagtagtcatgtatggaattgagaattgcagtataaagaaagctgagcaccgaagaattgatgcttttgaactgtggtgttggagaagactcttgagagtcccttggactgcaaggagatccaaccagtccattctgaaggagatcagccctgggatttctttgggag

SEQ ID NO: 150 - DNA, 243 bp, Bos taurus
caggactggaaaaggtcagttttcattccaatcccaaagaaaggcaatgccaaagaatgctcaaactaccgcacaattgtactcatctcacatgctagtaaagtaatgctcaaaattctccaggccaggcttcagcaatatgtgaaccatgaacttcctgatgttaaaggcagaagaaccagagatcaaattgccaacatccactggatcataaaaaaagcaagagagttccagaaaagcatc

SEQ ID NO: 151 - DNA, 260 bp, Bos taurus
gtgcaattgtgcggtagtttgagcattctttggcattgcctttctttgggattggaatgaaaactgaccttttccagtcctgtggccactgctgagtttccaaatttgctggcatattgagtgcagcactttcatagcatcatcttttaggatttgaaatagctcacctggaactccatcacctccgctacctttgtttgtagtgatgcttcctaaagcccacttgacttcacattccaggatgtctggatctaggtgag

SEQ ID NO: 152 - DNA, 237 bp, Bos taurus
caaatatattgagtcttttaaccaccatctccataaatggcattaaatccaccagtattggattccagctgagttatttcaaatcctagaagatgatgctgtgaaactgctgcactcaatatgccagcaaatttggaaaactcagcagtggccacaggactggaaaaggtcagttttcattccaatcccaaagaaaggcaatgccaaagaatgctcaaactactgcacaattgcact

SEQ ID NO: 153 - DNA, 289 bp, Bos taurus
gatctcctttaggatggactggttggatctccttgcagaacaagggactctcgagtcttttccaacaccgcagttcaaaagcatcaattctttggcactcagctttctttatagttcaactctcacatccatacatgaccactggaaaaaccatagccttgactagacggacctttattgacaaagtaatgtctctgcttttgaatatgctgtctaggttggtcataactttccttccaaggagtaagtgtcttttaatttcatggctgcagtcaccatctgcagtgat

SEQ ID NO: 154 - DNA, 266 bp, Bos taurus
atccaacggatgttggcaatttgatctctggttcctctgccttttctaaagccagcttgaacatctggaagttcagattcacgtactgctgaagcctggcttggagaattctgagcattactttactagcgtgtgaagtgaatgcaattgtgcggtagtttgagcattctttggcattgcctttctttgggattggaatgaaaactgaccttttccagtcctgtggccactgctgagttttccaaatttgctggcatattgagtgc

SEQ ID NO: 155 - DNA, 263 bp, Bos taurus
gcatcatctttcaggatttggaataggtcagttttcattccaatcccaaagaaaggcaatgccaaagaatgctcaaactactgcacaattgcactcatctcacacgctagtaaagtaatgctcaaaattctccaagccaggcttcagcaatatgtgaaccgtgaacttcctgatgttcaagctggcaggggaaccagagatcaaattgccaacatccgctggatcatggaaaaagcagagagttccagaaaaacatctatttc

SEQ ID NO: 156 - DNA, 225 bp, Bos taurus
agtacagccactatggagaacagcgtggagattccttaaaaaactggaaatagaactgccttatgatccagcaatcccactgctgggcatacacactgaggaaaccagaagggaaagagacacgtgtaccccaatgttcattgcagcactgttccaggacatggaagcaacctagatgtccatcagcagatgaatggataagaaagctgtggtacatatatacaa

SEQ ID NO: 157 - DNA, 200 bp, Bos taurus
gtacccattttttactagagagcgagattactggcttgactgctctctccccctttggactctcctttttctccaccaggtcgtctgtgtctcctccctaacccctctctactttacccaactctgtgaatttctgtgtgttccagatggtggggaacacttagggaactgattactggctggatctgtctcccttcttt

SEQ ID NO: 158 - DNA, 200 bp, Bos taurus
atcgcagcactgtttataatagccaggacatggaagcaacctagatgtccatcagcagatgaatggataagaaagctgtggtacatatacacaatggagtattactcagccattaaaaagaatacatttgaatcagttctaatgaggtggatgaaactggagcctattatacagagtgaagtaagccagaaaaaaaaaac

SEQ ID NO: 159 - DNA, 200 bp, Bos taurus
ttttccatgcaaatagggaccaaaagaaagcaggagtagcaatactcatatcagataaaatagactttaaaacaaaggctgtgaaaagagacaaagaaggtcactacataatgattaaaggatcaatccaagaagaagatataacaattataaatatatatgcacccaacacgggagcaccgcagtatgtaagacaaatg

SEQ ID NO: 160 - DNA, 200 bp, Bos taurus
ctagggatctcttcaaggaaatcagagataccaaaggaagatttcatgcaaagatgagctcgataaaggacaaaaatggtatggacctaacagacgcagaatatattaagaagagatggcaacaatacacagaagaatcatacaaaaaaggtcttcacgacccagataatcacgatggtgtgatcactgacctagagcca

SEQ ID NO: 161 - DNA, 200 bp, Bos taurus
gcatcgttcggaaaaaaccccaggttccaaatacagctcgacaagcggcctctctccccggggacatctcgagaggcaagcggagctccatgcctcaacccaagacgaggcctgactctcctgtccccagtctgcagggaccctgcgatcggagtctgaaatcagaggtaccctgcggttcctgcctcaactggagatga

SEQ ID NO: 162 - DNA, 200 bp, Bos taurus
aggtgggaggggcctctcgggacttttgggggtttggtgcattggaagagggcctcatctccagttgaggcaggaaccgcagggtacctctgatttcagactccgatcgcagggtccctgcagactggggacgggagagtcaggcctcgtcttgggttgaggcatgggactccgcttgcctctcgagatgtccccgggga

SEQ ID NO: 163 - DNA, 200 bp, Bos taurus
tgtttttatctcacgtaatgccataacaaaccacttgtggaatctttgttcctgactagagatcaagccctgaacctttggagtgggagcactgactccaagactatagcctaccagagagctaaccccagggagtatcaaatagcgggaactcacacaaaggaaagcacttgaaaaggacccagcgtcacccgccgcca

SEQ ID NO: 164 - DNA, 200 bp, Bos taurus
tcaacacgaaggggcagtgacccgcccgtgcatcgtccggaaaagacccccaggttccaaatacagctcgacaagtggcctctctccccggggacacctcgagaggcaagcggagttccatgcctcaacccaagacgaggcctgactctcctgtccccagtctgcagggaccctgcgatcggagtctgaaatcagaggaa